(19) United States
(12) Patent Application Publication
(10) Pub. No.: US 2022/0296619 A1
(43) Pub. Date: Sep. 22, 2022

(54) PHARMACEUTICAL FORMULATIONS OF TENOFOVIR ALAFENAMIDE

(71) Applicant: Gilead Sciences, Inc., Foster City, CA (US)

(72) Inventors: Jessica M. Bane, Ames, IA (US); Elham Nejati, San Mateo, CA (US); Dimitrios Stefanidis, Saratoga, CA (US)

(21) Appl. No.: 17/636,511

(22) PCT Filed: Aug. 18, 2020
(86) PCT No.: PCT/US2020/046758

§ 371 Date: Feb. 18, 2022

Related U.S. Application Data
(60) Provisional application No. 62/888,959, filed on Aug. 19, 2019.

(51) Int. Cl. A61K 31/675; A61K 31/573; A61K 47/34; A61K 47/38; A61K 47/26; A61K 9/10; A61K 9/00

(52) U.S. Cl. CPC A61K 31/675; A61K 31/573; A61K 47/34; A61K 47/38; A61K 47/26; A61K 9/10; A61K 9/0019

(57) ABSTRACT

Long-acting formulations comprising isopropyl ((S)—((((R)-1-(6-amino-9H-purin-9-yl)propan-2-yl)oxy)methyl)(phenoxy)phosphoryl)-L-alaninate, or a pharmaceutically acceptable salt thereof, and a biodegradable polymer, e.g. poly(lactic-co-glycolic acid) (PLGA), are described, as are methods of making the long acting formulations and uses thereof.

### CROSS-REFERENCE

This application claims priority to U.S. Provisional Patent Application No. 62/888,959, filed Aug. 19, 2020, which is incorporated here in entirety for all purposes.

### FIELD

Pharmaceutical formulations that may be used for treating or preventing human immunodeficiency virus (HIV) infection are described. In particular, long acting formulations of tenofovir alafenamide (TAF), methods for their preparation, and uses thereof as therapeutic or prophylactic agents are described.

### BACKGROUND

For certain patients, for example, those with difficult or limited access to health care, adherence to daily oral treatment or prophylactic regimens can be challenging. Drugs and formulations that offer favorable pharmaceutical properties (for example, improved potency, long-acting pharmacokinetics, low solubility, low clearance, and/or other properties) are amenable to less frequent administration and provide for better patient compliance.

### SUMMARY

Provided herein are pharmaceutical compositions comprising: (i) a compound of Formula I:

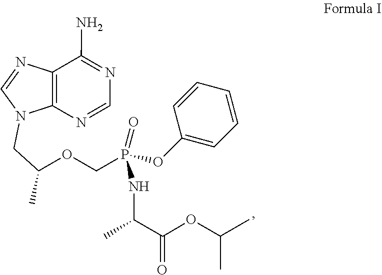 [SMILES: CC(C)OC(=O)[C@H](C)N[P@](=O)(CO[C@H](C)Cn1cnc2c(N)ncnc21)Oc1ccccc1]

or a pharmaceutically acceptable salt thereof, and (ii) a biodegradable polymer. In some embodiments, the compositions described herein consist essentially of the compound of Formula I, or the pharmaceutically acceptable salt thereof and the biodegradable polymer. In some embodiments, the compositions described herein consist of the compound of Formula I, or the pharmaceutically acceptable salt thereof and a biodegradable polymer.

In some embodiments, the pharmaceutical compositions comprise sucrose acetate isobutyrate in an amount less than 5% w/w. In some embodiments, the pharmaceutical compositions comprise sucrose acetate isobutyrate in an amount less than 0.5% w/w. In some embodiments, the pharmaceutical compositions comprise sucrose acetate isobutyrate in an amount less than 0.1% w/w. In some embodiments, the pharmaceutical compositions described herein do not comprise sucrose acetate isobutyrate. In some embodiments, the pharmaceutical compositions are free of sucrose acetate isobutyrate.

In some embodiments, the compositions described herein further comprise an additional therapeutic agent. In some embodiments, the additional therapeutic agent in an anti-inflammatory agent. In some embodiments, the anti-inflammatory agent is a steroid. In some embodiments the additional therapeutic agent is a corticosteroid. In some embodiments the additional therapeutic agent is a dexamethasone.

Also provided herein are pharmaceutical formulations comprising the compositions described herein and a suspending vehicle. In some embodiments, the suspending vehicle comprises (i) a suspending agent, (ii) a wetting agent, and (iii) a buffer.

Further provided herein are methods for treating a human immunodeficiency virus (HIV) infection, the methods comprising administering to a subject in need thereof a composition or a pharmaceutical formulation described herein.

Additionally provided herein are methods for treating an HBV infection, the methods comprising administering to a subject in need thereof a composition or a pharmaceutical formulation described herein.

Also described herein are methods of making the compositions described herein, the methods comprising: (i) mixing the compound of Formula I:

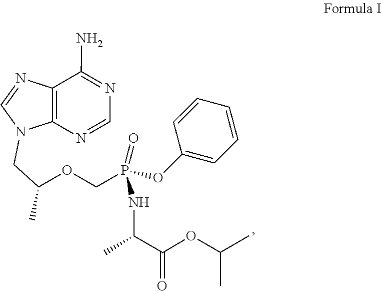 [SMILES: CC(C)OC(=O)[C@H](C)N[P@](=O)(CO[C@H](C)Cn1cnc2c(N)ncnc21)Oc1ccccc1]

or the pharmaceutically acceptable salt thereof, and a biodegradable polymer, and (ii) hot melt extrusion of a mixture comprising the compound of Formula I and the biodegradable polymer. There are also provided pharmaceutical compositions obtainable by these methods. There are also further provided pharmaceutical compositions prepared by hot melt extrusion.

### BRIEF DESCRIPTION OF THE DRAWINGS

FIG. 1. Shows a flow diagram for an exemplary method of making microspheres comprising the TAF drug substance and PLGA.

FIG. 2. Shows a flow diagram for an exemplary method of making spray-dried dispersions comprising the TAF drug substance and PLGA.

FIG. 3a. Shows SEM images of the three types of compositions ((i) microspheres, (ii) spray-dried dispersions, and (iii) hot melt extruded pharmaceutical compositions) descried in Example 1.

FIG. 3b. Shows the chemical stability of the three types of compositions ((i) microspheres, (ii) spray-dried dispersions, and (iii) hot melt extruded pharmaceutical compositions) descried in Example 1.

FIG. 4a. Shows a flow diagram for an exemplary method of making hot melt extruded pharmaceutical compositions comprising TAF and PLGA.

FIG. 4b. Shows overlaps of XRPD spectrums of the crystalline Form I of TAF free base starting material and two exemplary pharmaceutical formulations prepared by the hot-melt extrusion methods described herein (i) 30% TAF (free base (FB), crystalline Form I, and micronized) and 70% PLGA8515 and (ii) 30% TAF (free base (FB), crystalline Form I, and micronized) and 70% PLGA7525.

FIG. 5. Shows a comparison of the (i) chemical stability studies and (ii) dog PK studies of compositions comprising (a) 30% crystalline TAF bis-xinafoate (micronized) and 70% PLGA7525 and (b) 30% crystalline Form I of TAF free base (micronized) and 70% PLGA7525.

FIG. 6. Shows a comparison of the dog PK studies of compositions comprising (a) 30% crystalline Form I of TAF free base (micronized) and 70% PLGA7525 and (b) 30% crystalline TAF vanillate (micronized) and 70% PLGA7525.

FIG. 7. Shows a comparison of the dog PK studies of compositions comprising (a) 20% crystalline Form I of TAF free base (micronized) and 80% PLGA8515 and (b) 20% crystalline TAF sebacate Form I (micronized) and 80% PLGA8515.

FIG. 8. Shows the (i) chemical stability studies and (ii) dog PK studies on a composition comprising (a) 30% crystalline TAF orotate Form I (micronized) and 70% PLGA8515.

FIG. 9. Shows a comparison of the dog PK studies on compositions comprising (i) 30% TAF free base (crystalline Form I) and 70% PLGA5050 and (ii) TAF free base (amorphous) and 70% PLGA5050.

FIG. 10. Shows a comparison of the dog PK studies on compositions comprising (i) 30% TAF free base (crystalline Form I) and 70% PLGA5050 and (ii) TAF free base (amorphous) and 70% PLGA5050.

FIG. 11. Shows comparison of dog PK studies of two compositions (i) 30% crystalline Form I of TAF free base and 70% PLGA5050 and (ii)) 30% amorphous TAF free base and 70% PLGA5050.

FIG. 12. Shows a comparison of the (i) chemical stability and (ii) dog PK studies of compositions comprising (a) 30% crystalline TAF Form I of free base (unmicronized) and 70% PLGA7525 and (b) 30% crystalline Form I of TAF free base (micronized) and 70% PLGA7525.

FIG. 13. Shows a comparison of the (i) chemical stability and (ii) dog PK studies of compositions comprising (a) 40% crystalline Form I of TAF free base and 60% PLGA7525 with d₁₀=42 μm, d₅₀=101 μm, and d₉₀=202 μm and (b) 50% crystalline Form I of TAF free base and 50% PLGA7525 with d₁₀=6 μm, d₅₀=19 μm, and d₉₀=81 μm.

FIG. 14. Shows a comparison of the (i) chemical stability and (ii) dog PK studies of compositions comprising (a) 40% crystalline Form I of TAF free base and 60% PLGA7525 with d₁₀=42 μm, d₅₀=101 μm, and d₉₀=202 μm and (b) 50% crystalline Form I of TAF free base and 50% PLGA7525 with d₁₀=6 μm, d₅₀=19 μm, and d₉₀=81 μm.

FIG. 15. Shows a comparison of the (i) chemical stability and (ii) dog PK studies of compositions comprising (a) 40% crystalline Form I of TAF free base and 60% PLGA7525 with d₁₀=42 μm, d₅₀=101 μm, and d₉₀=202 μm and (b) 50% crystalline Form I of TAF free base and 50% PLGA7525 with d₁₀=6 μm, d₅₀=19 μm, and d₉₀=81 μm.

FIG. 16. Shows a comparison of the dog PK studies of two compositions (i) 20% TAF free base (crystalline Form I, micronized) and 80% PLGA8515 and (ii) 20% TAF free base (crystalline Form I, micronized) and 80% PLGA5050.

FIG. 17. Shows a comparison of the (i) chemical stability and (ii) dog PK studies on two compositions (i) 20% TAF (free base, crystalline Form I, micronized) and 80% PLGA7525 and (ii) 30% TAF (free base, crystalline Form I, micronized) and 70% PLGA7525.

FIG. 18. Shows a comparison of dog PK studies on two compositions (i) 20% TAF (free base, crystalline, micronized) and 80% PLGA7525 and (ii) 30% TAF (free base, crystalline, micronized) and 70% PLGA7525.

FIG. 19. Shows flow diagrams depicting exemplary alternative methods of making dexamethasone comprising pharmaceutical compositions described herein.

FIG. 20. Shows a comparison of dog PK studies for two formulations (i) 18.9% TAF (free base, crystalline, and micronized), 80.7% PLGA8515, and 0.4% dexamethasone and (ii) 19.0% TAF (free base, crystalline, and micronized), 80% PLGA8515.

FIG. 21. Shows a flow diagram depicting an exemplary method of making the suspending vehicles described herein.

FIG. 22. Shows the impact various suspending vehicles on the syringeability/injectability of the formulations described herein.

FIG. 23. Shows the impact of the suspending vehicle wetting agent on the syringeability of the pharmaceutical formulations.

FIGS. 24A and 24B. Show a comparison of dog PK studies for the following formulations (i) 20% TAF Sebacate (crystalline Form I, micronized) (non-sterile) and 80% PLGA8515, (ii) 20% TAF Sebacate (crystalline Form I, micronized) (sterile) and 80% PLGA8515, (iii) 35% TAF Sebacate (crystalline Form I, micronized) (sterile, gamma irradiated) and 65% PLGA8515, and (iv) 45% TAF Sebacate (crystalline Form I, micronized) (sterile, gamma irradiated) and 55% PLGA8515.

FIGS. 25A and 25B. Show a comparison of dog PK studies and clinical observations for the following formulations (i) 20% TAF Sebacate (crystalline Form I, micronized) (non-sterile) and 80% PLGA8515, (ii) 20% TAF Sebacate (crystalline, Form I, micronized) (sterile) and 80% PLGA8515, and (iii) 19.7% TAF Sebacate (crystalline, Form I, micronized) (sterile), 78.9% PLGA8515, and 1.4% methylprednisolone acetate.

FIGS. 26A and 26B. Show a comparison of dog PK studies and clinical observations for the following formulations (i) 35% TAF Sebacate (crystalline Form I, micronized) and 65% PLGA8515, (ii) 34.0% TAF Sebacate (crystalline, Form I, micronized) (sterile), 63.1% PLGA8515, and 2.9% methylprednisolone acetate (iii) 34.5% TAF Sebacate (crystalline, Form I, micronized), 64.0% PLGA8515, and 1.5% methylprednisolone acetate.

FIGS. 27A and 27B. Show a comparison of dog PK studies and clinical observations for the following formulations (i) 19% TAF free base (crystalline and micronized) and 81% PLGA8515, (ii) 18.5% TAF free base (crystalline and micronized), 79.1% PLGA8515, and 2.4% methylprednisolone acetate (iii) 20% TAF Sebacate (crystalline, Form I, micronized) (sterile) and 80% PLGA8515, and (iv) 19.7% TAF Sebacate (crystalline, Form I, micronized) (sterile), 78.9% PLGA8515, and 1.4% methylprednisolone acetate.

FIGS. 28A and 28B. Show a comparison of dog PK studies and clinical observations for the following formulations (i) 19.7% TAF Sebacate (crystalline and micronized), 78.9% PLGA8515, and 1.4% methylprednisolone acetate (ii) 34% TAF Sebacate (crystalline and micronized), 63.1% PLGA8515, and 2.9% methylprednisolone acetate, and (iii) 34.5% TAF Sebacate (crystalline and micronized), 64.0% PLGA8515, and 1.5% methylprednisolone acetate.

### DETAILED DESCRIPTION

In the following description, certain specific details are set forth in order to provide a thorough understanding of various embodiments described herein. However, one skilled in the art will understand that the embodiments described herein may be practiced without these details. The description below of several embodiments is made with the understanding that the present invention is to be considered as an exemplification of the claimed subject matter, and is not intended to limit the appended claims to the specific embodiments illustrated. The headings used throughout this description are provided for convenience only and are not to be construed to limit the claims in any way. Embodiments illustrated under any heading may be combined with embodiments illustrated under any other heading.

### i. DEFINITIONS

Unless the context requires otherwise, throughout the present description and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment described herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The d₉₀ values referred herein describe the size where ninety percent of particles in a sample have a smaller particle size than the specified d₉₀ value. For example, a d₉₀ of about 4 μm, means that 90% of the particles in the sample are smaller than 4 μm. Likewise, the d₅₀ values specified herein describe the size such that that 50% of particles in the sample are smaller than the specified d₅₀ value. Similarly, d₁₀ values listed herein are the size at which 10% of the particles in the sample have a size smaller than this value.

The term “TAF Drug substance” as used herein refers to tenofovir alafenamide (TAF) or a pharmaceutically acceptable salt thereof. TAF is a nucleotide reverse transcriptase inhibitor having the formula (WO2002/008241):

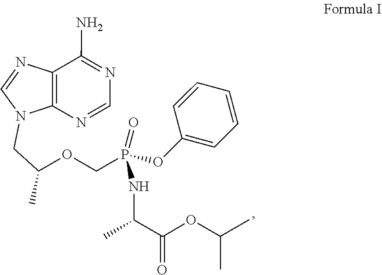 [SMILES: CC(C)OC(=O)[C@H](C)N[P@](=O)(CO[C@H](C)Cn1cnc2c(N)ncnc21)Oc1ccccc1]

Its IUPAC name is (S)-isopropyl-2-(((S)—((((R)-1-(6-amino-9H-purin-9-yl)propan-2-yl)oxy)methyl)(phenoxy)phosphoryl)amino)propanoate. It is also referred to as {9-[(R)-2-[[(S)—[[(S)-1-(isopropoxycarbonyl)ethyl]amino]phenoxyphosphinyl]-methoxy]propyl]adenine}. TAF is also referred to herein as the compound of Formula I.

Tenofovir alafenamide can be present in the compositions described herein in solvated and unsolvated form, and references to “tenofovir alafenamide” include both these forms. As used herein, and in absence of a specific reference to a particular pharmaceutically acceptable salt and/or solvate of tenofovir alafenamide, any dosages, whether expressed in e.g. milligrams or as % by weight, should be taken as referring to the amount of tenofovir alafenamide, i.e. the amount of:

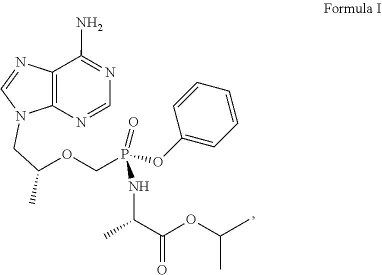 [SMILES: CC(C)OC(=O)[C@H](C)N[P@](=O)(CO[C@H](C)Cn1cnc2c(N)ncnc21)Oc1ccccc1]

For example, therefore, a reference to “25 mg tenofovir alafenamide or a pharmaceutically acceptable salt thereof” means an amount of tenofovir alafenamide or a pharmaceutically acceptable salt thereof which provides the same amount of tenofovir alafenamide as 25 mg of tenofovir alafenamide free base.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compositions may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

“Solvate” as used herein refers to an aggregate that comprises one or more molecules of a compound described herein with one or more molecules of solvent. The solvent may be water, in which case the solvate may be a hydrate. Alternatively, the solvent may be an organic solvent. Examples of organic solvents include, but are not limited to methanol, ethanol, acetonitrile, and dichloromethane.

The term “therapeutically effective amount” or “effective amount” of a composition or a compound or pharmaceutically acceptable salts, isomer, or a mixture thereof, described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition responsive to HIV activity. The therapeutically effective amount may vary depending on the subject, and the disease or condition being treated, the weight and age of the subject, the severity of the disease or condition, and the manner of administering, which can readily be determined by one of ordinary skill in the art.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, the term “about X” includes description of “X”.

The invention herein is also meant to encompass all pharmaceutically acceptable salts and/or co-crystals of tenofovir alafenamide being isotopically-labeled by having one or more atoms replaced by an atom having a different atomic mass or mass number. Examples of isotopes that can be incorporated into the described compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabeled compounds could be useful to help determine or measure the effectiveness of the compounds, by characterizing, for example, the site or mode of action, or binding affinity to pharmacologically important site of action. Certain isotopically-labeled salts and/or co-crystals of tenofovir alafenamide, for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled salts and/or co-crystals of tenofovir alafenamide can generally be prepared by conventional techniques known to those skilled in the art or by processes analogous to those described in the Examples as set out below using an appropriate isotopically-labeled reagent in place of the non-labeled reagent previously employed.

Crystalline forms may be characterized by the interlattice plane intervals determined by an X-ray powder diffraction pattern (XRPD). Unless otherwise stated, XRPD patterns referred to herein were collected using Kα1=1.5406 Å radiation.

### ii. OVERVIEW

The mechanism of cell loading by TAF is summarized in Antiviral Res. 125 (2016) 63-70 (Tenofovir Alafenamide: A novel prodrug of tenofovir for the treatment of human immunodeficiency virus, A. S. Ray et al.). In whole blood, TAF preferentially loads PBMC (peripheral blood mononuclear cells) over red blood cells. TAF enters cells passively where it is subject to ester hydrolysis by the lysosomal carboxypeptidase cathepsin A (CatA). Following chemical release of phenol from an unstable metabolite, a key intermediate metabolite is formed with alanine conjugated to TFV (tenofovir). Alanine is released either by enzymatic or chemical degradation to release TFV that is subsequently phosphorylated to the pharmacologically active metabolite TFV-DP (tenofovir-diphosphate). TFV is slowly released from cells into plasma where it is eliminated from the body renally.

In some embodiments, the pharmaceutical formulations provided herein maintain the therapeutically effective amount of TAF, TFV and/or TFV-DP in the plasma and/or PMBCs for an extended period of time, thereby acting as long acting formulations of TAF. For example, the pharmaceutical formulations provided herein maintain the therapeutically effective amount of TFV-DP in the PMBCs for an extended period of time.

In some embodiments, the pharmaceutical formulations provided herein maintain a therapeutically effective TFV-DP concentration in the PMBCs for at least one week or more, for example for at least two weeks, three weeks, 1 month, at least two months, or at least three months. In some embodiments, the pharmaceutical formulations provided herein maintain a therapeutically effective TFV-DP concentration in the PMBCs for about 1 month, about 2 month, about 3 months or more. The expression “a therapeutically effective TFV-DP concentration in the PMBCs” as used herein means that the level of the TFV-DP in the PMBCs is at least about 1 μM.

In some embodiments, after administration of the pharmaceutical formulations provided herein to a human subject, a PMBC of the human subject has a TFV-DP concentration of greater than 1 μM. In some embodiments, one week after administration to the human subject, a PMBC of the human subject has a TFV-DP concentration of greater than 1 μM. In some embodiments, two weeks after administration to a human subject, a PMBC of the human subject has a TFV-DP concentration of greater than 1 μM. In some embodiments, three weeks after administration to a human subject, a PMBC of the human subject has a TFV-DP concentration of greater than 1 μM. In some embodiments, one month after administration to a human subject, a PMBC of the human subject has a TFV-DP concentration of greater than 1 μM. In some embodiments, two month after administration to a human subject, a PMBC of the human subject has a TFV-DP concentration of greater than 1 μM. In some embodiments, three month after administration to a human subject, a PMBC of the human subject has a TFV-DP concentration of greater than 1 μM.

In some embodiments, the pharmaceutical formulations provided herein are suspensions for injections and are obtained by reconstitution of a pharmaceutical composition provided herein with a suspending vehicles provided herein.

### iii. PHARMACEUTICAL COMPOSITIONS

Provided herein are pharmaceutical compositions of the TAF drug substance. In various embodiments, the pharmaceutical compositions described herein comprise a TAF drug substance and a biodegradable polymer. In some embodiments, the pharmaceutical compositions described herein consist essentially of the TAF drug substance and the biodegradable polymer. In some embodiments, the pharmaceutical compositions described herein consist of the TAF drug substance and the biodegradable polymer.

In some embodiments, the combined amount of the TAF drug substance and the biodegradable polymer in the pharmaceutical compositions described herein is greater than about 95% of the total pharmaceutical composition weight, for example greater than about 96%, about 97%, about 98%, about 99%, about 99.5%, or about 99.9% of the total pharmaceutical composition weight.

In some embodiments, the combined amount of the TAF drug substance and the biodegradable polymer in the pharmaceutical compositions described herein is about 95-100% of the total pharmaceutical composition weight. For example, the amount of the TAF drug substance and the biodegradable polymer in the pharmaceutical compositions is about 96%-100%, about 97%-100%, about 98%-100%, about 99%-100%, about 95%-99%, about 96%-99%, about 97%-99%, about 98%-99%, about 95%-98%, about 96%-98%, about 97%-98%, about 98.5%-99.5%, about 95%-97%, or about 96%-97% of the total pharmaceutical composition weight.

In some embodiments, the pharmaceutical compositions described herein further comprise an additional therapeutic agent. In some embodiments, the additional therapeutic agent may be an agent to help alleviate the injection site reactions. In some embodiments, the additional therapeutic agent is an anti-inflammatory agent, for example a corticosteroid. In some embodiments the additional therapeutic agent is a dexamethasone. The combined amount of the TAF drug substance, the biodegradable polymer, and the additional therapeutic agent in the pharmaceutical compositions described herein is greater than about 95% of the total pharmaceutical composition weight, for example greater than about 96%, about 97%, about 98, about 99%, about 99.5%, or about 99.9% of the total pharmaceutical composition weight. In some embodiments, the total amount of the TAF drug substance, the biodegradable polymer, and the additional therapeutic agent in the pharmaceutical compositions described herein is about 95-100% of the total pharmaceutical composition weight. For example, the amount of the TAF drug substance, the biodegradable polymer, and the additional therapeutic agent in the pharmaceutical compositions is about 96%-100%, about 97%-100%, about 98%-100%, about 99%-100%, about 95%-99%, about 96%-99%, about 97%-99%, about 98%-99%, about 95%-98%, about 96%-98%, about 97%-98%, 98.5%-99.5%, about 95%-97%, or about 96%-97% of the total pharmaceutical composition weight.

a. TAF Drug Substance

In some embodiments, the TAF drug substance is the compound of Formula I. In some embodiments, the TAF drug substance is a pharmaceutically acceptable salt of the compound of Formula I. The TAF drug substance can be crystalline, amorphous, or a combination thereof. In some embodiments, the TAF drug substance is crystalline. In some embodiments, the TAF drug substance is amorphous.

In some embodiments, the TAF drug substance is a pharmaceutically acceptable salt of the compound of Formula I. In some embodiments, the pharmaceutically acceptable salt is a salt with an acid, for example, hydrochloric acid, sulfuric acid, persulfate, thiocyanate, hydrobromic, hydroiodic, phosphoric, nitric, carbonic, lauryl sulfuric acid, glycerophosphate, methanesulfonamide acid, ethanesulfonic acid, 2-hydroxyethanesulfonic acid, taurine, camphorsulfonic acid, cyclohexyl sulfamic acid, sulfamic acid, ethanedisulfonic acid, succinic acid, benzenesulfonic acid, p-toluenesulfonic acid, p-hydroxybenzoic acid, o-hydroxybenzoic acid, 2,5-dihydroxybenzoic acid, sulfanilic acid, naphthalene-2-sulfonic acid, naphthalene-1,5-disulfonic acid, formic acid, acetic acid, glycolic acid, 2,2-dichloroacetic acid, propionic acid, L-lactic acid, D-lactic acid, racemic lactic acid (aka: DL-lactic acid), cyclopentane propionic acid, butyric acid, valeric acid, hexanoic acid, heptanoic acid, octanoic acid, nonanoic acid, decanoic acid, undecanoic acid, lauric acid, palmitic acid, stearic acid, oleic acid, oxalic acid, malonic acid, succinic acid, L-malic acid, D-malic acid, racemic malic acid (aka: DL-malic acid), L-tartaric acid, D-tartaric acid, racemic acid (aka: DL-tartrate), meso-tartaric acid, maleic acid, hydroxy maleic acid, glutaric acid, 2-oxo-glutaric acid, adipic acid, sebacic acid, citric acid Benzoic acid, p-methoxybenzoic acid, 4-acetamido-benzoic acid, salicylic acid, acetylsalicylic acid, gentisic acid, 4-aminosalicylic acid, phenylacetic acid, L-mandelic acid, D-mandelic acid, racemic mandelic acid (aka: DL-mandelic acid), 3-phenylpropionic acid, cinnamic acid, caffeic acid, phenylbutyric acid, picric acid, nicotinic acid, orotic acid, quinic acid, ascorbic acid, glucuronic acid, gluconic acid, galacturonic acid, glucoheptonic acid, lactobionic acid, camphorsulfonic acid, galacturonic acid (also known as: mucic acid), tannic acid (also known as: tannic acid), alginic acid, hydroxynaphthoic acid (also known as: 3-hydroxy-2-naphthoic acid), pamoic acid (also known as: 4,4′-methylenebis (3-hydroxy-2-naphthoic acid), or Pu acid), acylated amino acid or amino acid (e.g., acetylamino acid, hippuric acid, aspartic acid, glutamic acid, pyroglutamic acid, glutamine, asparagine, etc.).

In some embodiments, the pharmaceutically acceptable salt is a salt with an organic acid such as acetic acid, oxalic acid, fumaric acid, citric acid, succinic acid, tartaric acid, salicylic acid, benzoic acid, glycolic acid, methane sulfonic acid, ethanesulfonic acid, benzenesulfonic acid, p-toluenesulfonic acid, lactic acid, maleic acid, malonic acid, malic acid, isethionic acid, lactobionic acid, mandelic acid, p-coumaric acid, ferulic acid, sinapic acid, caffeic acid, chlorogenic acid, caftaric acid, coutaric acid, p-hydroxy benzoic acid, vanillic acid, syringic acid, 4-(4-phenoxybenzoyl) benzoic acid, gentisic acid, protocatechuic acid, gallic acid, lipoic acid, aspartic acid and the like; or an inorganic acid such as hydrochloric acid, hydrobromic acid, sulfuric acid, phosphoric acid, nitric acid, sulfamic acid and the like.

In some embodiments, the pharmaceutically acceptable salt of the compound of Formula I is selected from the group consisting of tenofovir alafenamide fumarate, tenofovir alafenamide ferulate, tenofovir alafenamide phosphate, tenofovir alafenamide succinate, tenofovir alafenamide citrate, tenofovir alafenamide tartarate, tenofovir alafenamide lactate, tenofovir alafenamide mesylate, tenofovir alafenamide hemifumarate, tenofovir alafenamide sesquifumarate, tenofovir alafenamide oxalate, tenofovir alafenamide malonate, tenofovir alafenamide L-malate, tenofovir alafenamide saccharin, tenofovir alafenamide mucate, tenofovir alafenamide maleate, tenofovir alafenamide hydrochloride, tenofovir alafenamide ethanesulfonate, tenofovir alafenamide benzenesulfonate, tenofovir alafenamide methanesulfonate, tenofovir alafenamide sulfate, tenofovir alafenamide hydrochloride, tenofovir alafenamide sesquifumarate p-toluene sulfonate, tenofovir alafenamide hemipamoate, tenofovir alafenamide sebacate, tenofovir alafenamide napsylate, tenofovir alafenamide orotate, tenofovir alafenamide vanillate, and tenofovir alafenamide bis-xinafoate.

In some embodiments, the pharmaceutically acceptable salt of the compound of Formula I is selected from the group consisting of tenofovir alafenamide hemipamoate, tenofovir alafenamide sebacate, tenofovir alafenamide napsylate, tenofovir alafenamide orotate, tenofovir alafenamide vanillate, and tenofovir alafenamide bis-xinafoate.

In some embodiments, the pharmaceutically acceptable salt of the compound of Formula I, is tenofovir alafenamide orotate, tenofovir alafenamide vanillate, tenofovir alafenamide sebacate, or tenofovir alafenamide bis-xinafoate.

In some embodiments, the TAF drug substance is selected from the group consisting of tenofovir alafenamide free base, tenofovir alafenamide hemipamoate, tenofovir alafenamide sebacate, tenofovir alafenamide napsylate, tenofovir alafenamide orotate, tenofovir alafenamide vanillate, and tenofovir alafenamide bis-xinafoate. In some embodiments, the TAF drug substance is selected from the group consisting of tenofovir alafenamide free base, tenofovir alafenamide orotate, tenofovir alafenamide vanillate, tenofovir alafenamide sebacate, and tenofovir alafenamide bis-xinafoate.

In some embodiments, the TAF drug substance is tenofovir alafenamide vanillate. In some embodiments, the TAF drug substance is a crystalline form of tenofovir alafenamide vanillate. In some embodiments, the TAF drug substance is amorphous tenofovir alafenamide vanillate. In some embodiments, the TAF drug substance is the crystalline form of tenofovir alafenamide vanillate as described in U.S. Pat. No. 10,287,307. In certain embodiments, the TAF drug substance is the crystalline form of tenofovir alafenamide vanillate, wherein the crystalline tenofovir alafenamide vanillate has an XRPD pattern comprising degree 2θ-reflections (+/−0.2 degrees 2θ) at 6.6°, 9.3°, and 22.8°. In certain embodiments, the TAF drug substance is the crystalline form of tenofovir alafenamide vanillate, wherein the crystalline tenofovir alafenamide vanillate has an XRPD pattern comprising degree 2θ-reflections (+/−0.2 degrees 2θ) at 6.6°, 9.3°, 14.2°, 15.2°, 19.0°, 19.8°, and 22.8°. In certain embodiments, the TAF drug substance is the crystalline form of tenofovir alafenamide vanillate, wherein the crystalline tenofovir alafenamide vanillate has an XRPD pattern comprising degree 2θ-reflections (+/−0.2 degrees 2θ) at 6.6°, 9.3° 10.8°, 12.3°, 14.2°, 15.2°, 18.4°, 19.0°, 19.8°, 22.1°, 22.8°, 25.0°, and 32.4°.

In some embodiments, the TAF drug substance is tenofovir alafenamide orotate. In some embodiments, the TAF drug substance is a crystalline form of tenofovir alafenamide orotate. In some embodiments, the TAF drug substance is amorphous tenofovir alafenamide orotate. In some embodiments, the TAF drug substance is the crystalline Form I of tenofovir alafenamide orotate as described in U.S. Pat. No. 10,287,307. In some embodiments, the TAF drug substance is the crystalline Form I of tenofovir alafenamide orotate, wherein the Form I has an XRPD pattern comprising degree 2θ-reflections (+/−0.2 degrees 2θ) at 3.0°, 3.5°, and 8.9°. In some embodiments, the TAF drug substance is the crystalline Form I of tenofovir alafenamide orotate, wherein the Form I has an XRPD pattern comprising degree 2θ-reflections (+/−0.2 degrees 2θ) at 3.0°, 5.9°, 8.9°, and 11.8°. In some embodiments, the TAF drug substance is the crystalline Form I of tenofovir alafenamide orotate, wherein the Form I has an XRPD pattern comprising degree 2θ-reflections (+/−0.2 degrees 2θ) at 3.0°, 5.9°, 8.9°, 11.8°, 14.8°, 16.0°, 17.7°, 18.7°, and 21.5°. In some embodiments, the TAF drug substance is the crystalline Form I of tenofovir alafenamide orotate, wherein the Form I has an XRPD pattern comprising degree 2θ-reflections (+/−0.2 degrees 2θ) at 3.0°, 3.5°, 5.9°, 8.9°, 11.8°, 14.8°, 16.0°, 17.7°, 18.7°, 21.5°, 27.2°, 28.7°, and 31.5°.

In some embodiments, the TAF drug substance is the crystalline Form II of tenofovir alafenamide orotate as described in U.S. Pat. No. 10,287,307. In some embodiments, the TAF drug substance is the crystalline Form II of tenofovir alafenamide orotate, wherein the Form II has an XRPD pattern comprising degree 2θ-reflections (+/−0.2 degrees 2θ) at 3.4°, 3.8°, and 13.8°. In some embodiments, the TAF drug substance is the crystalline Form II of tenofovir alafenamide orotate, wherein the Form II has an XRPD pattern comprising degree 2θ-reflections (+/−0.2 degrees 2θ) at 3.4°, 3.8°, 6.9°, 10.3°, and 13.8°. In some embodiments, the TAF drug substance is the crystalline Form II of tenofovir alafenamide orotate, wherein the Form II has an XRPD pattern comprising degree 2θ-reflections (+/−0.2 degrees 2θ) at 3.4°, 6.9°, 10.3°, 13.8°, 15.4°, 17.3°, 19.0°, 22.8° and 29.0°. In some embodiments, the TAF drug substance is the crystalline Form II of tenofovir alafenamide orotate, wherein the Form II has an XRPD pattern comprising degree 2θ-reflections (+/−0.2 degrees 2θ) at 3.4°, 6.9°, 10.3°, 13.8°, 15.4°, 17.3°, 18.4°, 19.0°, 21.6° 22.8°, and 29.0°.

In some embodiments, the TAF drug substance is the crystalline Form III of tenofovir alafenamide orotate as described in U.S. Pat. No. 10,287,307. In some embodiments, the TAF drug substance is the crystalline Form III of tenofovir alafenamide orotate, wherein the Form III has an XRPD pattern comprising degree 2θ-reflections (+/−0.2 degrees 2θ) at 3.8°, 15.7°, and 19.0°. In some embodiments, the TAF drug substance is the crystalline Form III of tenofovir alafenamide orotate, wherein the Form III has an XRPD pattern comprising degree 2θ-reflections (+/−0.2 degrees 2θ) at 3.8°, 9.4°, 12.4°, 15.7°, and 19.0°. In some embodiments, the TAF drug substance is the crystalline Form III of tenofovir alafenamide orotate, wherein the Form III has an XRPD pattern comprising degree 2θ-reflections (+/−0.2 degrees 2θ) at 3.8°, 8.3°, 9.4°, 12.4°, 15.7°, 16.4°, 19.0°, 24.5°, 26.6° and 28.9°. In some embodiments, the TAF drug substance is the crystalline Form III of tenofovir alafenamide orotate, wherein the Form III has an XRPD pattern comprising degree 2θ-reflections (+/−0.2 degrees 2θ) at 3.8°, 6.9°, 8.3°, 9.4°, 12.4°, 15.7°, 16.4°, 19.0°, 22.8°, 24.5°, 26.6°, 27.6°, and 28.9°.

In some embodiments, the TAF drug substance is tenofovir alafenamide bis-xinafoate. In some embodiments, the TAF drug substance is amorphous tenofovir alafenamide bis-xinafoate. In some embodiments, the TAF drug substance is a crystalline form of tenofovir alafenamide bis-xinafoate. In some embodiments, the TAF drug substance is the crystalline form of tenofovir alafenamide bis-xinafoate as described in U.S. Pat. No. 10,287,307. In some embodiments, the TAF drug substance is crystalline form of tenofovir alafenamide bis-xinafoate, wherein the crystalline form of tenofovir alafenamide bis-xinafoate has an XRPD pattern comprising degree 2θ-reflections (+/−0.2 degrees 2θ) at 4.5°, 8.9°, 14.4°, and 15.4°. In some embodiments, the TAF drug substance is crystalline form of tenofovir alafenamide bis-xinafoate, wherein the crystalline form of tenofovir alafenamide bis-xinafoate has an XRPD pattern comprising degree 2θ-reflections (+/−0.2 degrees 2θ) at 14.4°, 15.4°, and 21.7°. In some embodiments, the crystalline form of tenofovir alafenamide bis-xinafoate has an XRPD pattern comprising degree 2θ-reflections (+/−0.2 degrees 2θ) at 4.5°, 8.9°, 11.2°, 14.4°, 15.4°, 18.8°, 21.7°, and 25.5°. In some embodiments, the crystalline form of tenofovir alafenamide bis-xinafoate has an XRPD pattern comprising degree 2θ-reflections (+/−0.2 degrees 2θ) at 4.5°, 7.7°, 8.9°, 11.2°, 13.4°, 14.4°, 14.7°, 15.4°, 15.7°, 17.0°, 18.3°, 18.8°, 21.7°, 21.9°, 25.5°, 25.9°, 32.9°, 33.8°, and 36.5°

In some embodiments, the TAF drug substance is tenofovir alafenamide sebacate. In some embodiments, the TAF drug substance is amorphous tenofovir alafenamide sebacate. In some embodiments, the TAF drug substance is a crystalline form of tenofovir alafenamide sebacate. In some embodiments, the TAF drug substance is the crystalline Form I of tenofovir alafenamide sebacate as described in U.S. Pat. No. 10,287,307. In some embodiments, the crystalline Form I of tenofovir alafenamide sebacate, has an XRPD pattern comprising degree 2θ-reflections (+/−0.2 degrees 2θ) at 6.6°, 9.4°, and 9.6°. In some embodiments, the crystalline Form I of tenofovir alafenamide sebacate, has an XRPD pattern comprising degree 2θ-reflections (+/−0.2 degrees 2θ) at 5.3°, 6.6°, 9.4°, 9.6°, and 19.8°. In some embodiments, crystalline tenofovir alafenamide sebacate Form I has an XRPD pattern comprising degree 2θ-reflections (+/−0.2 degrees 2θ) at 5.3°, 6.6°, 9.4°, 9.6°, 14.8°, 15.7°, 18.7°, 19.3°, 19.8°, and 22.1°. In some embodiments, the crystalline Form I of tenofovir alafenamide sebacate has an XRPD pattern comprising degree 2θ-reflections (+/−0.2 degrees 2θ) at 5.3°, 6.6°, 9.4°, 9.6°, 14.8°, 15.7°, 18.7°, 19.3°, 19.8°, and 22.1°. In some embodiments, crystalline Form I of tenofovir alafenamide sebacate has an XRPD pattern comprising degree 2θ-reflections (+/−0.2 degrees 2θ) at 5.3°, 6.6°, 9.4°, 9.6°, 11.7°, 12.6°, 14.8°, 15.7°, 18.7°, 19.3°, 19.8°, 20.9°, 22.1°, 23.4°, 23.8°, 26.2°, 28.2°, and 29.0°.

In some embodiments, the TAF drug substance is tenofovir alafenamide napsylate. In some embodiments, the TAF drug substance is amorphous tenofovir alafenamide napsylate. In some embodiments, the TAF drug substance is crystalline tenofovir alafenamide napsylate. In some embodiments, the TAF drug substance is crystalline Form I of tenofovir alafenamide napsylate as described in U.S. Pat. No. 10,287,307. In some embodiments, the crystalline Form I of tenofovir alafenamide napsylate is characterized by an XRPD pattern comprising degree 2θ-reflections (+/−0.2 degrees 2θ) at 7.8°, 19.2°, and 27.2°. In some embodiments, the crystalline Form I of tenofovir alafenamide napsylate is characterized by an XRPD pattern comprising degree 2θ-reflections (+/−0.2 degrees 2θ) at 3.9°, 7.8°, 13.6°, 15.3°, and 19.2°. In some embodiments, the crystalline Form I of tenofovir alafenamide napsylate is characterized by an XRPD pattern comprising degree 2θ-reflections (+/−0.2 degrees 2θ) at 3.9°, 7.8°, 13.6°, 15.3°, 19.2°, 19.4°, 19.8°, 20.6°, 23.8° and 27.2°. In some embodiments, the crystalline Form I of tenofovir alafenamide napsylate is characterized by an XRPD pattern comprising degree 2θ-reflections (+/−0.2 degrees 2θ) at 3.9°, 7.8°, 9.8°, 13.2°, 13.6°, 15.3°, 15.5°, 16.5°, 17.8°, 19.2°, 19.4°, 19.8°, 20.6°, 23.0°, 23.8°, 24.1°, 26.0°, and 27.2°.

In some embodiments, the TAF drug substance is tenofovir alafenamide hemipamoate. In some embodiments, the TAF drug substance is amorphous tenofovir alafenamide hemipamoate. In some embodiments, the TAF drug substance is crystalline tenofovir alafenamide hemipamoate. In some embodiments, the TAF drug substance is crystalline Form I of tenofovir alafenamide hemipamoate as described in U.S. Pat. No. 10,287,307. In some embodiments, the crystalline Form I of tenofovir alafenamide hemipamoate is characterized by an XRPD pattern comprising degree 2θ-reflections (+/−0.2 degrees 2θ) at 14.8°, 19.0°, and 22.3°. In some embodiments, the crystalline Form I of tenofovir alafenamide hemipamoate is characterized by an XRPD pattern comprising degree 2θ-reflections (+/−0.2 degrees 2θ) at 7.4°, 8.4°, 10.6°, 14.8°, and 22.3°. In some embodiments, the crystalline Form I of tenofovir alafenamide hemipamoate is characterized by an XRPD pattern comprising degree 2θ-reflections (+/−0.2 degrees 2θ) at 7.4°, 8.4°, 10.6°, 14.8°, 22.3°, 19.0°, 25.7°, 20.1°, 23.8° and 17.4°. In some embodiments, the crystalline Form I of tenofovir alafenamide hemipamoate is characterized by an XRPD pattern comprising degree 2θ-reflections (+/−0.2 degrees 2θ) at 7.4°, 8.4°, 10.6°, 11.2°, 13.1°, 13.8°, 14.8°, 15.8°, 17.4°, 19.0°, 20.1°, 21.0°, 22.3°, 23.8°, 25.7°, 28.8°, 30.6°, and 32.9°.

In some embodiments, the TAF drug substance is crystalline Form II of tenofovir alafenamide hemipamoate as described in U.S. Pat. No. 10,287,307. In some embodiments, the crystalline Form II of tenofovir alafenamide hemipamoate is characterized by an XRPD pattern comprising degree 2θ-reflections (+/−0.2 degrees 2θ) at 5.5°, 22.1°, and 23.2°. In some embodiments, the crystalline Form II of tenofovir alafenamide hemipamoate is characterized by an XRPD pattern comprising degree 2θ-reflections (+/−0.2 degrees 2θ) at 5.5°, 10.9°, 16.2°, 22.1°, and 23.2°. In some embodiments, the crystalline Form II of tenofovir alafenamide hemipamoate is characterized by an XRPD pattern comprising degree 2θ-reflections (+/−0.2 degrees 2θ) at 5.5°, 10.9°, 16.2°, 22.1°, 23.2°, 24.1°, 27.6°, and 29.0°.

In some embodiments, the TAF drug substance is the free base form of tenofovir alafenamide. In some embodiments, the TAF drug substance is amorphous tenofovir alafenamide free base. In some embodiments, the TAF drug substance is a crystalline form of tenofovir alafenamide free base. In some embodiments, the TAF drug substance is a crystalline form of tenofovir alafenamide free base as described in U.S. Pat. No. 7,803,788 (crystalline Form I of tenofovir alafenamide free base), which is characterized by the following X-ray data:

| | |
|---|---|
| Crystal Dimensions | 0.25 × 0.12 × 0.08 mm |
| Crystal System | orthorhombic |
| Lattice Type | Primitive |
| Lattice Parameters | a = 8.352(1) Å |
| | b = 15.574(2) Å |
| | c = 18.253(2) Å |
| | V = 2374.2(5) Å³ |
| Space Group | P2₁2₁2₁(#19) |
| Z value | 4 |
| D_{calc} | 1.333 g/cm³ |
| F₀₀₀ | 1008.00 |
| μ(MoKα) | 1.60 cm⁻¹ |

In some embodiments, the crystalline Form I of tenofovir alafenamide free base is characterized by an XRPD pattern comprising degree 2θ-reflections (+/−0.2 degrees 2θ) at 11.3°, 19.6°, and 22.4°. In some embodiments, the crystalline Form I of tenofovir alafenamide free base is characterized by an XRPD pattern comprising degree 2θ-reflections (+/−0.2 degrees 2θ) at 11.3°, 19.6°, 21.3°, and 22.4°. In some embodiments, the crystalline Form I of tenofovir alafenamide free base is characterized by an XRPD pattern comprising degree 2θ-reflections (+/−0.2 degrees 2θ) at 7.4°, 11.3°, 19.6°, 21.3° and 22.4°. In some embodiments, the crystalline Form I of tenofovir alafenamide free base is characterized by an XRPD pattern comprising degree 2θ-reflections (+/−0.2 degrees 2θ) at 7.4°, 11.3°, 12.9°, 17.6°, 19.6°, 21.3°, 22.4°, 24.5°, 25.6°, and 26.9°. In some embodiments, the crystalline Form I of tenofovir alafenamide free base is characterized by an XRPD pattern comprising degree 2θ-reflections (+/−0.2 degrees 2θ) at 7.4°, 9.7°, 11.3°, 12.9°, 14.3°, 17.6°, 18.3°, 19.6°, 21.3°, 22.4°, 24.5°, 25.6°, 26.9°, 28.9°, 30.0° and 31.9°. In some embodiments, the crystalline Form I of tenofovir alafenamide free base is characterized by XRPD peaks as shown below:

| Pos. [° 2Th.] | Rel. Int. [%] |
|---|---|
| 7.4 | 33 |
| 9.7 | 7 |
| 11.3 | 79 |
| 12.9 | 16 |
| 14.3 | 7 |
| 17.6 | 20 |
| 18.3 | 10 |
| 19.6 | 100 |
| 20.6 | 10 |
| 21.3 | 55 |
| 22.4 | 59 |
| 24.5 | 16 |
| 25.6 | 12 |
| 26.9 | 12 |
| 28.9 | 6 |
| 30.0 | 8 |
| 31.9 | 7 |

In some embodiments, the TAF drug substance is a micronized form. In some embodiments, the micronized form has a d₉₀ of less than 50 μm. For example, the micronized form has a d₉₀ of less than 45 μm, 40 μm, 35 μm, 30 μm, 25 μm, 20 μm, 15 μm, 10 μm, 9 μm, 8 μm, 7 μm, 6 μm, 5 μm, 4 μm, 3 μm, 2 μm, or 1 μm. In some embodiments, the micronized form has a d₉₀ of about 1 μm-50 μm, for example, about 1 μm-45 μm, 1 μm-40 μm, 1 μm-35 μm, 1 μm-30 μm, 1 μm-25 μm, 1 μm-20 μm, 1 μm-15 μm, 1 μm-10 μm, 1 μm-9 μm, 1 μm-8 μm, 1 μm-7 μm, 1 μm-6 μm, 1 μm-5 μm, 1 μm-4 μm, 1 μm-3 μm, or 1 μm-2 μm. In some embodiments, the micronized form has a d₉₀ of ≤about 10 μm. In some embodiments, the micronized form has a d₉₀ of about 1 μm-10 μm. In some embodiments, the micronized form has a d₉₀ of about 1 μm-5 μm. In some embodiments, the micronized form has a d₉₀ of about 4 μm.

In some embodiments, the micronized form has a d₅₀ of less than 30 μm. For example, the micronized form has a d₅₀ of less than 25 μm, 20 μm, 15 μm, 10 μm, 9 μm, 8 μm, 7 μm, 6 μm, 5 μm, 4 μm, 3 μm, 2 μm, or 1 μm. In some embodiments, the micronized form has a d₅₀ of about 1 μm-30 μm, for example, about 1 μm-25 μm, 1 μm-20 μm, 1 μm-15 μm, 1 μm-10 μm, 1 μm-9 μm, 1 μm-8 μm, 1 μm-7 μm, 1 μm-6 μm, 1 μm-5 μm, 1 μm-4 μm, 1 μm-3 μm, or 1 μm-2 μm. In some embodiments, the micronized form has a d₅₀ of about 1 μm-10 μm. In some embodiments, the micronized form has a d₅₀ of about 1 μm-5 μm. In some embodiments, the micronized form has a d₅₀ of about 4 μm, 3 μm, 2 μm, or 1 μm. In some embodiments, the micronized form has a d₅₀ of about 2 μm.

In some embodiments, the micronized form has a d₁₀ of less than 20 μm. For example, the micronized form has a d₁₀ of less than 15 μm, 10 μm, 9 μm, 8 μm, 7 μm, 6 μm, 5 μm, 4 μm, 3 μm, 2 μm, 1 μm, 0.5 μm, 0.4 μm, 0.3 μm, 0.2 μm, or 0.1 μm. In some embodiments, the micronized form has a d₁₀ of about 0.1 μm-20 μm, for example, about 1 μm-20 μm, 1 μm-15 μm, 1 μm-10 μm, 1 μm-9 μm, 1 μm-8 μm, 1 μm-7 μm, 1 μm-6 μm, 1 μm-5 μm, 1 μm-4 μm, 1 μm-3 μm, 1 μm-2 μm, 0.1 μm-15 μm, 0.1 μm-10 μm, 0.1 μm-9 μm, 0.1 μm-8 μm, 0.1 μm-7 μm, 0.1 μm-6 μm, 0.1 μm-5 μm, 0.1 μm-4 μm, 0.1 μm-3 μm, 0.1 μm-2 μm, or 0.1 μm-1 μm. In some embodiments, the micronized form has a d₁₀ of about 0.1 μm-10 μm. In some embodiments, the micronized form has a d₁₀ of about 0.1 μm-5 μm. In some embodiments, the micronized form has a d₁₀ of about 0.5 μm-5 μm. In some embodiments, the micronized form has a d₁₀ of about 4 μm, 3 μm, 2 μm, 1 μm, 0.9 μm, 0.8 μm, 0.7 μm, 0.6 μm, 0.5 μm, 0.4 μm, 0.3 μm, 0.2 μm, or 0.1 μm. In some embodiments, the micronized form has a d₁₀ of about 1 μm.

In some embodiments, the micronized form has a d₉₀ of about 5 μm-15 μm, a d₅₀ of about 1 μm-10 μm, and a d₁₀ of about 0.5 μm-5 μm. In some embodiments, the micronized form has a d₉₀ of about 1 μm-10 μm, a d₅₀ of about 1 μm-5 μm, and a d₁₀ of about 0.1 μm-2 μm. In some embodiments, the micronized form has a d₉₀ of about 4 μm, a d₅₀ of about 2 μm, and a d₁₀ of about 1 μm.

In some embodiments, the compound of Formula I is not micronized (also referred to as non-micronized or unmicronized).

Any amount of the TAF drug substance can be present in the pharmaceutical compositions described herein. In some embodiments, the amount of the TAF drug substance present is about 5% to about 50% of the total weight of the pharmaceutical composition. In some embodiments, the amount of the TAF drug substance present is about 5 to about 45% of the total pharmaceutical composition weight. In some embodiments, the amount of the TAF drug substance present is about 15% to about 45%, about 15% to about 40%, about 15% to about 35%, about 15% to about 30%, about 15% to about 25% or about 15% to about 20% of the total pharmaceutical composition weight.

In some embodiments, the amount of the TAF drug substance present is about 15% to about 45% of the total pharmaceutical composition weight. In some embodiments, the amount of the TAF drug substance present is about 15% to about 35% of the total pharmaceutical composition weight. In some embodiments, the amount of the TAF drug substance present is about 29% to about 31% of the total pharmaceutical composition weight. In some embodiments, the amount of the TAF drug substance present is about 29% of the total pharmaceutical composition weight. In some embodiments, the amount of the TAF drug substance present is about 30% of the total pharmaceutical composition weight. In some embodiments, the amount of the TAF drug substance present is about 31% of the total pharmaceutical composition weight.

In some embodiments, the amount of the TAF drug substance is about 18% to about 20% of the total pharmaceutical composition weight. In some embodiments, the amount of the TAF drug substance present is about 18% of the total pharmaceutical composition weight. In some embodiments, the amount of the TAF drug substance present is about 19% of the total pharmaceutical composition weight. In some embodiments, the amount of the TAF drug substance present is about 20% of the total pharmaceutical composition weight.

In some embodiments, the TAF drug substance is the TAF free base and the amount of the TAF drug substance present is 15%-45% of the total pharmaceutical composition weight. In some embodiments, the TAF drug substance is the TAF free base and the amount of the TAF drug substance present is 18%-20% of the total pharmaceutical composition weight. In some embodiments, the TAF drug substance is the TAF free base and the amount of the TAF drug substance present is about 19%-20% of the total pharmaceutical composition weight. In some embodiments, the TAF drug substance is the TAF free base and the amount of the TAF drug substance present is about 18%-19% of the total pharmaceutical composition weight. In some embodiments, the TAF drug substance is the TAF free base and the amount of the TAF drug substance present is 18.5%-19.5% of the total pharmaceutical composition weight. In some embodiments, the TAF drug substance is the TAF free base and the amount of the TAF drug substance present is about 19% of the total pharmaceutical composition weight.

In some embodiments, the TAF drug substance is the TAF free base and the amount of the TAF drug substance present is 29%-31% of the total pharmaceutical composition weight. In some embodiments, the TAF drug substance is the TAF free base and the amount of the TAF drug substance present is 29%-30% of the total pharmaceutical composition weight. In some embodiments, the TAF drug substance is the TAF free base and the amount of the TAF drug substance present is 30%-31% of the total pharmaceutical composition weight. In some embodiments, the TAF drug substance is the TAF free base and the amount of the TAF drug substance present is 29.5%-30.5% of the total pharmaceutical composition weight. In some embodiments, the TAF drug substance is the TAF free base and the amount of the TAF drug substance present is 30% of the total pharmaceutical composition weight.

In some embodiments, the TAF drug substance is a crystalline form of the TAF free base and the amount of the TAF drug substance present is 15%-45% of the total pharmaceutical composition weight. In some embodiments, the TAF drug substance is a crystalline form of the TAF free base and the amount of the TAF drug substance present is 18%-20% of the total pharmaceutical composition weight. In some embodiments, the TAF drug substance is a crystalline form of the TAF free base and the amount of the TAF drug substance present is about 19%-20% of the total pharmaceutical composition weight. In some embodiments, the TAF drug substance is a crystalline form of the TAF free base and the amount of the TAF drug substance present is about 18%-19% of the total pharmaceutical composition weight. In some embodiments, the TAF drug substance is a crystalline form of the TAF free base and the amount of the TAF drug substance present is 18.5%-19.5% of the total pharmaceutical composition weight. In some embodiments, the TAF drug substance is a crystalline form of the TAF free base and the amount of the TAF drug substance present is about 19% of the total pharmaceutical composition weight.

In some embodiments, the TAF drug substance is a crystalline form of the TAF free base and the amount of the TAF drug substance present is 29%-31% of the total pharmaceutical composition weight. In some embodiments, the TAF drug substance is a crystalline form of the TAF free base and the amount of the TAF drug substance present is 29%-30% of the total pharmaceutical composition weight. In some embodiments, the TAF drug substance is a crystalline form of the TAF free base and the amount of the TAF drug substance present is 30%-31% of the total pharmaceutical composition weight. In some embodiments, the TAF drug substance is a crystalline form of the TAF free base and the amount of the TAF drug substance present is 29.5%-30.5% of the total pharmaceutical composition weight. In some embodiments, the TAF drug substance is a crystalline form of the TAF free base and the amount of the TAF drug substance present is 30% of the total pharmaceutical composition weight.

In some embodiments, the TAF drug substance is crystalline Form I of the TAF free base and the amount of the TAF drug substance present is 15%-45% of the total pharmaceutical composition weight. In some embodiments, the TAF drug substance is crystalline Form I of the TAF free base and the amount of the TAF drug substance present is 18%-20% of the total pharmaceutical composition weight. In some embodiments, the TAF drug substance is crystalline Form I of the TAF free base and the amount of the TAF drug substance present is about 19%-20% of the total pharmaceutical composition weight. In some embodiments, the TAF drug substance is crystalline Form I of the TAF free base and the amount of the TAF drug substance present is about 18%-19% of the total pharmaceutical composition weight. In some embodiments, the TAF drug substance is crystalline Form I of the TAF free base and the amount of the TAF drug substance present is 18.5%-19.5% of the total pharmaceutical composition weight. In some embodiments, the TAF drug substance is crystalline Form I of the TAF free base and the amount of the TAF drug substance present is about 19% of the total pharmaceutical composition weight.

In some embodiments, the TAF drug substance is crystalline Form I of the TAF free base and the amount of the TAF drug substance present is 29%-31% of the total pharmaceutical composition weight. In some embodiments, the TAF drug substance is crystalline Form I of the TAF free base and the amount of the TAF drug substance present is 29%-30% of the total pharmaceutical composition weight. In some embodiments, the TAF drug substance is crystalline Form I of the TAF free base and the amount of the TAF drug substance present is 30%-31% of the total pharmaceutical composition weight. In some embodiments, the TAF drug substance is crystalline Form I of the TAF free base and the amount of the TAF drug substance present is 29.5%-30.5% of the total pharmaceutical composition weight. In some embodiments, the TAF drug substance is crystalline Form I of the TAF free base and the amount of the TAF drug substance present is 30% of the total pharmaceutical composition weight.

In some embodiments, the TAF drug substance is tenofovir alafenamide sebacate and the amount of the TAF drug substance present is 15%-45% of the total pharmaceutical composition weight. In some embodiments, the TAF drug substance is tenofovir alafenamide sebacate and the amount of the TAF drug substance present is 18%-20% of the total pharmaceutical composition weight. In some embodiments, the TAF drug substance is tenofovir alafenamide sebacate and the amount of the TAF drug substance present is about 19%-20% of the total pharmaceutical composition weight. In some embodiments, the TAF drug substance is tenofovir alafenamide sebacate and the amount of the TAF drug substance present is about 18%-19% of the total pharmaceutical composition weight. In some embodiments, the TAF drug substance is tenofovir alafenamide sebacate and the amount of the TAF drug substance present is 18.5%-19.5% of the total pharmaceutical composition weight. In some embodiments, the TAF drug substance is a tenofovir alafenamide sebacate and the amount of the TAF drug substance present is about 19% of the total pharmaceutical composition weight. In some embodiments, the TAF drug substance is a tenofovir alafenamide sebacate and the amount of the TAF drug substance present is about 20% of the total pharmaceutical composition weight. In some embodiments, the TAF drug substance is a tenofovir alafenamide sebacate and the amount of the TAF drug substance present is about 33-37% of the total pharmaceutical composition weight. In some embodiments, the TAF drug substance is a tenofovir alafenamide sebacate and the amount of the TAF drug substance present is about 35% of the total pharmaceutical composition weight. In some embodiments, the TAF drug substance is a tenofovir alafenamide sebacate and the amount of the TAF drug substance present is about 43-47% of the total pharmaceutical composition weight. In some embodiments, the TAF drug substance is a tenofovir alafenamide sebacate and the amount of the TAF drug substance present is about 45% of the total pharmaceutical composition weight.

In some embodiments, the TAF drug substance is tenofovir alafenamide sebacate and the amount of the TAF drug substance present is 29%-31% of the total pharmaceutical composition weight. In some embodiments, the TAF drug substance is tenofovir alafenamide sebacate and the amount of the TAF drug substance present is 29%-30% of the total pharmaceutical composition weight. In some embodiments, the TAF drug substance is tenofovir alafenamide sebacate and the amount of the TAF drug substance present is 30%-31% of the total pharmaceutical composition weight. In some embodiments, the TAF drug substance is tenofovir alafenamide sebacate and the amount of the TAF drug substance present is 29.5%-30.5% of the total pharmaceutical composition weight. In some embodiments, the TAF drug substance is tenofovir alafenamide sebacate and the amount of the TAF drug substance present is 30% of the total pharmaceutical composition weight.

In some embodiments, the TAF drug substance is a crystalline form of tenofovir alafenamide sebacate and the amount of the TAF drug substance present is 15%-45% of the total pharmaceutical composition weight. In some embodiments, the TAF drug substance is a crystalline form of tenofovir alafenamide sebacate and the amount of the TAF drug substance present is 18%-20% of the total pharmaceutical composition weight. In some embodiments, the TAF drug substance is a crystalline form of tenofovir alafenamide sebacate and the amount of the TAF drug substance present is about 19%-20% of the total pharmaceutical composition weight. In some embodiments, the TAF drug substance is a crystalline form of tenofovir alafenamide sebacate and the amount of the TAF drug substance present is about 18%-19% of the total pharmaceutical composition weight. In some embodiments, the TAF drug substance is a crystalline form of tenofovir alafenamide sebacate and the amount of the TAF drug substance present is 18.5%-19.5% of the total pharmaceutical composition weight. In some embodiments, the TAF drug substance is a crystalline form of tenofovir alafenamide sebacate and the amount of the TAF drug substance present is about 19% of the total pharmaceutical composition weight. In some embodiments, the TAF drug substance is a crystalline form of tenofovir alafenamide sebacate and the amount of the TAF drug substance present is about 20% of the total pharmaceutical composition weight. In some embodiments, the TAF drug substance is a crystalline form of tenofovir alafenamide sebacate and the amount of the TAF drug substance present is about 33-37% of the total pharmaceutical composition weight. In some embodiments, the TAF drug substance is a crystalline form of tenofovir alafenamide sebacate and the amount of the TAF drug substance present is about 35% of the total pharmaceutical composition weight. In some embodiments, the TAF drug substance is a crystalline form of tenofovir alafenamide sebacate and the amount of the TAF drug substance present is about 43-47% of the total pharmaceutical composition weight. In some embodiments, the TAF drug substance is a crystalline form of tenofovir alafenamide sebacate and the amount of the TAF drug substance present is about 45% of the total pharmaceutical composition weight.

In some embodiments, the TAF drug substance is a crystalline form of tenofovir alafenamide sebacate and the amount of the TAF drug substance present is 29%-310% of the total pharmaceutical composition weight. In some embodiments, the TAF drug substance is a crystalline form of tenofovir alafenamide sebacate and the amount of the TAF drug substance present is 29%-30% of the total pharmaceutical composition weight. In some embodiments, the TAF drug substance is a crystalline form of tenofovir alafenamide sebacate and the amount of the TAF drug substance present is 30%-31% of the total pharmaceutical composition weight. In some embodiments, the TAF drug substance is a crystalline form of tenofovir alafenamide sebacate and the amount of the TAF drug substance present is 29.5%-30.5% of the total pharmaceutical composition weight. In some embodiments, the TAF drug substance is a crystalline form of tenofovir alafenamide sebacate and the amount of the TAF drug substance present is 30% of the total pharmaceutical composition weight.

In some embodiments, the TAF drug substance is the crystalline Form I of tenofovir alafenamide sebacate and the amount of the TAF drug substance present is 15%-45% of the total pharmaceutical composition weight. In some embodiments, the TAF drug substance is the crystalline Form I of tenofovir alafenamide sebacate and the amount of the TAF drug substance present is 18%-20% of the total pharmaceutical composition weight. In some embodiments, the TAF drug substance is the crystalline Form I of tenofovir alafenamide sebacate and the amount of the TAF drug substance present is about 19%-20% of the total pharmaceutical composition weight. In some embodiments, the TAF drug substance is the crystalline Form I of tenofovir alafenamide sebacate and the amount of the TAF drug substance present is about 18%-19% of the total pharmaceutical composition weight. In some embodiments, the TAF drug substance is the crystalline Form I of tenofovir alafenamide sebacate and the amount of the TAF drug substance present is 18.5%-19.5% of the total pharmaceutical composition weight. In some embodiments, the TAF drug substance is the crystalline Form I of tenofovir alafenamide sebacate and the amount of the TAF drug substance present is about 19% of the total pharmaceutical composition weight. In some embodiments, the TAF drug substance is the crystalline Form I of tenofovir alafenamide sebacate and the amount of the TAF drug substance present is about 20% of the total pharmaceutical composition weight. In some embodiments, the TAF drug substance is the crystalline Form I of tenofovir alafenamide sebacate and the amount of the TAF drug substance present is about 33-37% of the total pharmaceutical composition weight. In some embodiments, the TAF drug substance is the crystalline Form I of tenofovir alafenamide sebacate and the amount of the TAF drug substance present is about 35% of the total pharmaceutical composition weight. In some embodiments, the TAF drug substance is the crystalline Form I of tenofovir alafenamide sebacate and the amount of the TAF drug substance present is about 43-47% of the total pharmaceutical composition weight. In some embodiments, the TAF drug substance is the crystalline Form I of tenofovir alafenamide sebacate and the amount of the TAF drug substance present is about 45% of the total pharmaceutical composition weight.

In some embodiments, the TAF drug substance is the crystalline Form I of tenofovir alafenamide sebacate and the amount of the TAF drug substance present is 29%-31% of the total pharmaceutical composition weight. In some embodiments, the TAF drug substance is the crystalline Form I of tenofovir alafenamide sebacate and the amount of the TAF drug substance present is 29%-30% of the total pharmaceutical composition weight. In some embodiments, the TAF drug substance is the crystalline Form I of tenofovir alafenamide sebacate and the amount of the TAF drug substance present is 30%-31% of the total pharmaceutical composition weight. In some embodiments, the TAF drug substance is the crystalline Form I of tenofovir alafenamide sebacate and the amount of the TAF drug substance present is 29.5%-30.5% of the total pharmaceutical composition weight. In some embodiments, the TAF drug substance is the crystalline Form I of tenofovir alafenamide sebacate and the amount of the TAF drug substance present is 30% of the total pharmaceutical composition weight.

b. Biodegradable Polymer

In various embodiments, the pharmaceutical compositions described herein comprise a biodegradable polymer. “Biodegradable polymers” are polymers that may be hydrolytically, enzymatically, or otherwise cleaved in vivo, resulting in degradation products which can be cleared from the body after administration. Examples of suitable polymers include but are not limited to PLGA, poly(D,L-lactide) (PDLLA), or PEG-PLGA.

In some embodiments, the biodegradable polymer present in the pharmaceutical compositions described herein is PLGA. The PLGA present in the pharmaceutical compositions described herein can have any L/G (lactic acid/glycolic acid) molar ratio. In some example, the PLGA present in the pharmaceutical compositions described herein has a L/G ratio of 50:50, 60:40, 65:35, 70:30, 75:25, 85:15, or 90:10. In some example, the PLGA present in the pharmaceutical compositions described herein has a L/G ratio of 75:25 (75% lactic acid and 25% glycolic acid) or 85:15 (85% lactic acid and 15% glycolic acid). In some example, the PLGA present in the pharmaceutical compositions described herein has a L/G ratio of 75:25. In some example, the PLGA present in the pharmaceutical compositions described herein has a L/G ratio of 85:15.

In some example, the PLGA present in the pharmaceutical compositions described herein has a L/G ratio of 50:50. In some embodiments, the PLGA present in the pharmaceutical compositions described herein has a L/G ratio of 60:40. In some embodiments, the PLGA present in the pharmaceutical compositions described herein has a L/G ratio of 65:35. In some embodiments, the PLGA present in the pharmaceutical compositions described herein has a L/G ratio of 75:25. In some embodiments, the PLGA present in the pharmaceutical compositions described herein has a L/G ratio of 70:30. In some embodiments, the PLGA present in the pharmaceutical compositions described herein has a L/G ratio of 85:15. In some embodiments, the PLGA present in the pharmaceutical compositions described herein has a L/G ratio of 90:10.

In some embodiments, the PLGA present in the pharmaceutical compositions described herein has an inherent viscosity of about 0.2 to about 0.8 dL/g. In some embodiments, the PLGA present in the compositions described herein has an inherent viscosity of about 0.20-0.75 dL/g, 0.20-0.70 dL/g, 0.20-0.65 dL/g, 0.20-0.60 dL/g, 0.20-0.55 dL/g, about 0.20-0.50 dL/g, 0.20-0.45 dL/g, 0.20-0.40 dL/g, 0.20-0.35 dL/g, 0.20-0.30 dL/g, 0.20-0.25 dL/g, 0.25-0.80 dL/g, 0.25-0.75 dL/g, 0.25-0.70 dL/g, 0.25-0.65 dL/g, 0.25-0.60 dL/g, 0.25-0.55 dL/g, about 0.25-0.50 dL/g, 0.25-0.45 dL/g, 0.25-0.40 dL/g, 0.25-0.35 dL/g, 0.25-0.30 dL/g, 0.30-0.80 dL/g, 0.30-0.75 dL/g, 0.30-0.70 dL/g, 0.30-0.65 dL/g, 0.30-0.60 dL/g, 0.30-0.55 dL/g, about 0.30-0.50 dL/g, 0.30-0.45 dL/g, 0.30-0.40 dL/g, 0.30-0.35 dL/g, 0.35-0.80 dL/g, 0.35-0.75 dL/g, 0.35-0.70 dL/g, 0.35-0.65 dL/g, 0.35-0.60 dL/g, 0.35-0.55 dL/g, about 0.35-0.50 dL/g, 0.35-0.45 dL/g, 0.35-0.40 dL/g, 0.40-0.80 dL/g, 0.40-0.75 dL/g, 0.40-0.70 dL/g, 0.40-0.65 dL/g, 0.40-0.60 dL/g, 0.40-0.55 dL/g, about 0.40-0.50 dL/g, 0.40-0.45 dL/g, 0.45-0.80 dL/g, 0.45-0.75 dL/g, 0.45-0.70 dL/g, 0.45-0.65 dL/g, 0.45-0.60 dL/g, 0.45-0.55 dL/g, about 0.45-0.50 dL/g, 0.50-0.80 dL/g, 0.50-0.75 dL/g, 0.50-0.70 dL/g, 0.50-0.65 dL/g, 0.50-0.60 dL/g, 0.50-0.55 dL/g, 0.55-0.80 dL/g, 0.55-0.75 dL/g, 0.55-0.70 dL/g, 0.55-0.65 dL/g, 0.55-0.60 dL/g, 0.60-0.80 dL/g, 0.60-0.75 dL/g, 0.60-0.70 dL/g, 0.60-0.65 dL/g, 0.65-0.80 dL/g, 0.65-0.75 dL/g, 0.65-0.70 dL/g, 0.70-0.80 dL/g, 0.70-0.75 dL/g, and 0.75-0.80 dL/g. In some embodiments, the PLGA present in the pharmaceutical compositions described herein has an inherent viscosity of about 0.25 to 0.70 dL/g. In some embodiments, the PLGA present in the pharmaceutical compositions described herein has an inherent viscosity about of 0.30 to 0.65 dL/g. In some embodiments, the PLGA present in the pharmaceutical compositions described herein has an inherent viscosity of about 0.3 to 0.6 dL/g.

In some embodiments, the PLGA present in the pharmaceutical compositions described herein has an L/G ratio of 75:25 or 85:15 and an inherent viscosity of about 0.3 to 0.6 dL/g. In some embodiments, the PLGA present in the pharmaceutical compositions described herein has an L/G ratio of 75:25 and an inherent viscosity of about 0.3 to 0.6 dL/g. In some embodiments, the PLGA present in the pharmaceutical compositions described herein has an L/G ratio of 85:15 and an inherent viscosity of about 0.3 to 0.6 dL/g.

In some embodiments, the PLGA present in the pharmaceutical compositions described herein has an L/G ratio of 75:25 and an inherent viscosity of about 0.3 dL/g or about 0.4 dL/g. In some embodiments, the PLGA present in the pharmaceutical compositions described herein has an L/G ratio of 75:25 and an inherent viscosity of about 0.4 dL/g. In some embodiments, the PLGA present in the pharmaceutical compositions described herein has an L/G ratio of 75:25 and an inherent viscosity of about 0.3 dL/g. In some embodiments, the PLGA present in the pharmaceutical compositions described herein has an L/G ratio of 85:15 and an inherent viscosity of about 0.6 dL/g. In some embodiments, the PLGA present in the pharmaceutical compositions described herein has an L/G ratio of 85:15 and an inherent viscosity of about 0.3 dL/g or about 0.4 dL/g. In some embodiments, the PLGA present in the pharmaceutical compositions described herein has an L/G ratio of 85:15 and an inherent viscosity of about 0.3 dL/g. In some embodiments, the PLGA present in the pharmaceutical compositions described herein has an L/G ratio of 85:15 and an inherent viscosity of about 0.4 dL/g. In some embodiments, the PLGA present in the pharmaceutical compositions described herein has an L/G ratio of 85:15 and an inherent viscosity of about 0.3 dL/g or about 0.65 dL/g.

In some embodiments, the biodegradable polymer present in the pharmaceutical compositions described herein is Poly(D,L-lactide) (PDLLA). In some embodiments, the biodegradable polymer used is PDLLA having an inherent viscosity of about 0.2 to 0.8 dL/g (e.g. about 0.25 dL/g-0.65 dL/g). In some embodiments, the biodegradable polymer used is PDLLA having an inherent viscosity of about 0.3 dL/g

In some embodiments, the biodegradable polymer used in the pharmaceutical compositions described herein is polyethylene glycol-poly lactic acid-co-glycolic acid (PEG-PLGA). In some embodiments, the biodegradable polymer is PEG-PLGA7525. In some embodiments, the biodegradable polymer used is PEG-PLGA7525 having an inherent viscosity of about 0.2 to about 0.8 dL/g (e.g. about 0.25 to about 0.65 dL/g). PEG can be of any molecular weight. In some embodiments, the PEG has a molecular weight of about 1,000-20,000 daltons. In some embodiments, the molecular weight of PEG is about 1,000-10,000. In some embodiments, the molecular weight of PEG is about 5,000 daltons. In some embodiments, the biodegradable polymer used is PEG-PLGA7525 having an inherent viscosity of about 0.6 dL/g.

In some embodiments, the biodegradable polymer is PEG-PLGA7525. In some embodiments, the biodegradable polymer used is PEG-PLGA8515 having an inherent viscosity of about 0.2 to about 0.8 dL/g (e.g. about 0.25 to about 0.65 dL/g). PEG can be of any molecular weight. In some embodiments, the PEG has a molecular weight of about 1,000-20,000 daltons. In some embodiments, the molecular weight of PEG is about 1,000-10,000. In some embodiments, the molecular weight of PEG is about 5,000 daltons.

The pharmaceutical compositions described herein are generally physically and/or chemically stable. As such the pharmaceutical compositions described herein can be stored at room temperature or refrigerated conditions for an extended period of time without significant degradation and/or change in physical form. In some embodiments, the pharmaceutical compositions can be stored at room temperature for at least one month, for example at least two months, at least three months, at least four months, at least five months, at least six months, at one year, or at least two years.

In some embodiments, the pharmaceutical compositions provided herein can be stored at 25° C. and 60% relative humidity for at least about one month without any significant change in the physical form of the TAF drug substance and without any chemical degradation of the TAF drug substance into impurities, wherein the impurities are selected from PMPA (phosphonate 9-R-(2-phosphonomethoxypropyl)adenine), PMPA anhydride, monophenyl PMPA, PMPA monoamidite, and phenol. In some embodiments, the pharmaceutical compositions provided herein can be stored at 25° C. and 60% relative humidity for at least about two months, about three months, about six months, about one year, or about 2 years without any significant change in the physical form of the TAF drug substance and without any chemical degradation of the TAF drug substance into impurities, wherein the impurities are selected from PMPA, PMPA anhydride, monophenyl PMPA, PMPA monoamidite, and phenol.

In some embodiments, the pharmaceutical compositions provided herein can be stored at 30° C. and 75% relative humidity for at least about one month without any significant change in the physical form of the TAF drug substance and without any chemical degradation of the TAF drug substance into impurities, wherein the impurities are selected from PMPA, PMPA anhydride, monophenyl PMPA, PMPA monoamidite, and phenol. In some embodiments, the pharmaceutical compositions provided herein can be stored at 30° C. and 75% relative humidity for at least about two months, about three months, about six months, about one year, or about 2 years without any significant change in the physical form of the TAF drug substance and without any chemical degradation of the TAF drug substance into impurities, wherein the impurities are selected from PMPA, PMPA anhydride, monophenyl PMPA, PMPA monoamidite, and phenol.

In some embodiments, after storage at 30° C. and 75% relative humidity for at least about one month the amount of impurities formed in the pharmaceutical compositions described herein is less than 0.5% (w/w), for example less than 0.4%, 0.3%, 0.2%, or 0.1%. In some embodiments, after storage at 30° C. and 75% relative humidity for at least about two months, about three months, about six months, about one year, or about 2 years the amount of impurities formed in the pharmaceutical compositions described herein is less than 0.1% (w/w), for example less than 0.8%, 0.5%, 0.4%, 0.3%, 0.2%, or 0.1%.

In some embodiments, the pharmaceutical compositions provided herein can be stored at 40° C. and 75% relative humidity for at least about one month without any significant change in the physical form of the TAF drug substance and without any chemical degradation of the TAF drug substance into impurities, wherein the impurities are selected from PMPA, PMPA anhydride, monophenyl PMPA, PMPA monoamidite, and phenol. In some embodiments, the pharmaceutical compositions provided herein can be stored at 40° C. and 75% relative humidity for at least about two months, about three months, about six months, about one year, or about 2 years without any significant change in the physical form of the TAF drug substance and without any chemical degradation of the TAF drug substance into impurities, wherein the impurities are selected from PMPA, PMPA anhydride, monophenyl PMPA, PMPA monoamidite, and phenol.

In some embodiments, after storage at 40° C. and 75% relative humidity for at least about one month the amount of impurities formed in the pharmaceutical compositions described herein is less than 1.5% (w/w), for example less than 1.0%, 0.8%, 0.6%, 0.4%, 0.3%, 0.2%, or 0.1%. In some embodiments, after storage at 30° C. and 75% relative humidity for at least about two months, about three months, about six months, about one year, or about 2 years the amount of impurities formed in the pharmaceutical compositions described herein is less than 3.0% (w/w), for example less than 2.5%, 2.0%, 1.5%, 1.0%, 0.8%, 0.5%, 0.4%, 0.3%, 0.2%, or 0.1%. The impurities can be measured by any relevant method, for example by HPLC.

In some embodiments, storage for about one month at a temperature of about 30° C. and a relative humidity of 75% results in less than 0.5% (w/w) impurities, wherein the impurities comprises PMPA, PMPA anhydride, monophenyl PMPA, PMPA monoamidite, and/or phenol. In some embodiments, storage for about one month at a temperature of about 30° C. and a relative humidity of 75% results in about 0.25% to about 0.45% (w/w) impurities, wherein the impurities comprises PMPA, PMPA anhydride, monophenyl PMPA, PMPA monoamidite, and/or phenol. The impurities can be measured by any relevant method, for example by HPLC.

### iv. METHOD OF MAKING THE PHARMACEUTICAL COMPOSITIONS

### Hot Melt Extrusion

Also provided herein are methods of making the pharmaceutical compositions described herein. In some embodiments, the methods of making the compositions described herein comprise hot melt extrusion of the TAF drug substance and the biodegradable polymer. In some embodiments, the methods of making the pharmaceutical compositions described herein comprise (i) forming a mixture of the TAF drug substance and the biodegradable polymer and (ii) hot melt extrusion of the mixture comprising the TAF drug substance and the biodegradable polymer to yield the pharmaceutical composition.

In some embodiments, the methods of making the pharmaceutical compositions described herein further comprise delumping the TAF drug substance before mixing with the biodegradable polymer. In some embodiments, the method additionally comprise cryomilling and/or vacuum drying the biodegradable polymer before mixing with the TAF drug substance. In some embodiments, the methods further comprise blending the mixture of TAF drug substance and biodegradable polymer.

In some embodiments, additional agent can be included in the mixture comprising the TAF drug substance and the biodegradable polymer (prior to the holt melt extrusion). In some embodiments, the additional agent is selected from glycerol monostearate, lecithin, cholesterol, and a therapeutic agent. In some embodiments, the therapeutic agent is an anti-inflammatory agent. In some embodiments, the anti-inflammatory agent is a steroid, for example a corticosteroid. In some embodiments, the anti-inflammatory agent is dexamethasone. In some embodiments, the additional agent is glycerol monostearate. In some embodiments, the additional agent is lecithin. In some embodiments, the additional agent is cholesterol. In some embodiments, the additional agent is a therapeutic agent. In some embodiments, the additional agent is an anti-inflammatory agent. In some embodiments, the additional agent is a steroid. In some embodiments, the additional agent is a corticosteroid. In some embodiments, the additional agent is dexamethasone. In some embodiments, the additional agent is a glucocorticoid. In some examples, the additional agent is methylprednisolone or methylprednisolone acetate (e.g. Depo-Medrol®).

The mixture comprising the TAF drug substance and the biodegradable polymer to be subjected to the hot-melt extrusion may be a slurry, solid, suspension, liquid, powdered or other such feed comprising the TAF drug substance and the biodegradable polymer. In some embodiments the hot melt extrusion is conducted on a dry (solid or powdered) feed comprising the TAF drug substance and biodegradable polymer.

The hot-melt extrusion process employed in some embodiments of the invention is conducted at an elevated temperature, i.e. the heating zone(s) of the extruder is above room temperature (about 20° C.). In some embodiments an operating temperature range that will minimize the degradation or decomposition of the TAF drug substance and/or the biodegradable polymer is selected. The operating temperature range is generally in the range of from about 20° C. to about 160° C. as determined by the setting for the extruder heating zone(s), for example about 30° C.-160° C., 40° C.-160° C., 50° C.-160° C., 60° C.-160° C., 70° C.-160° C., 80° C.-160° C., 90° C.-160° C., 100° C.-160° C., 110° C.-160° C., 120° C.-160° C., 130° C.-160° C., 140° C.-160° C., 150° C.-160° C., 20° C.-140° C., 30° C.-140° C., 40° C.-140° C., 50° C.-140° C., 60° C.-140° C., 70° C.-140° C., 80° C.-140° C., 90° C.-140° C., 100° C.-140° C., 110° C.-140° C., 120° C.-140° C., 130° C.- 140° C., 20° C.-120° C., 30° C.-120° C., 40° C.-120° C., 50° C.-120° C., 60° C.-120° C., 70° C.-120° C., 80° C.-120° C., 90° C.-120° C., 100° C.-120° C., 110° C.-120° C., 20° C.-100° C., 30° C.-100° C., 40° C.-100° C., 50° C.-100° C., 60° C.-110° C., 70° C.-100° C., 80° C.-100° C., 90° C.-100° C., 20° C.-80° C., 30° C.-80° C., 40° C.-80° C., 50° C.-80° C., 60° C.-80° C., 70° C.-80° C., 20° C.-60° C., 30° C.-60° C., 40° C.-60° C., 50° C.-60° C., 20° C.-40° C., or 30° C.-40° C. In some embodiments, the operating temperature range is from about 25° C. to about 95° C. In some embodiments, the operating temperature range is from about 50° C. to about 95° C. In some embodiments, the operating temperature range is from about 60° C. to about 85° C. In some embodiments, the operating temperature range is from about 67° C. to about 82° C., for example about 70° C. to about 80° C., about 65° C. to about 77° C., about 70° C. to about 75° C., about 70° C. to about 80° C., or about 70° C. to about 77° C.

The hot-melt extrusion process comprised in the methods descried here is generally described as follows. A suitable amount of a powdered TAF drug substance is mixed with the biodegradable polymer. Optionally, the TAF drug substance is delumped before being mixed with the biodegradable polymer. Further optionally, the biodegradable polymer may be cryomilled and/or vacuum dried before being mixed with the TAF drug substance. In some embodiments, additional components (for e.g. an additional therapeutic agent) may be added. The mixture is then placed in the extruder hopper and passed through the heated area of the extruder at a temperature which will melt or soften the biodegradable polymer, to form a matrix throughout which the TAF drug substance is dispersed. The molten or softened mixture then exits via a die, or other such element (which may be referred to as the extrudate). In some embodiments, the extrudate may optionally be shaped, molded, chopped, ground, milled, cryomilled, molded, sphegonized into beads, cut into strands, tableted or otherwise processed to obtain the pharmaceutical compositions of the desired physical form.

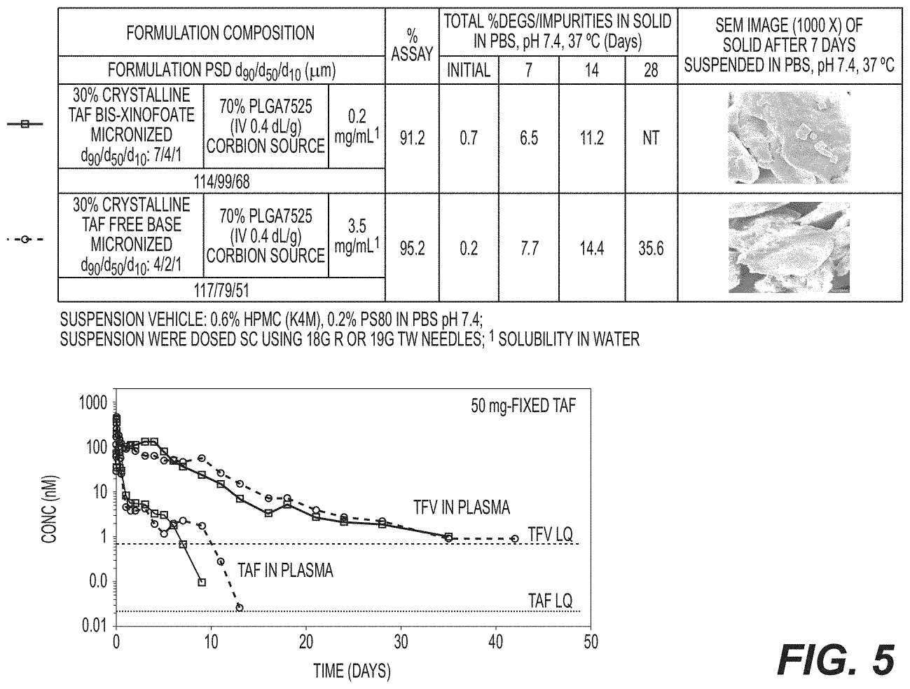

A particular advantage of the methods comprising hot melt extrusion described herein is that when crystalline TAF drug substance is used, the crystalline nature of the TAF drug substance is essentially retained throughout the process. As such, the resulting extrudate and/or the pharmaceutical composition is comprised of the crystalline TAF drug substance. Such pharmaceutical compositions are often characterized by improved chemical stability as compared to comparable pharmaceutical compositions comprising amorphous TAF drug substance (see e.g. FIG. 5b). By contrast, the methods of preparing the microspheres and the spray-dried dispersions described below, generally result in loss of the crystalline nature of the TAF drug substance.

Any extruder may be used in the methods described herein. In some embodiments, the extruder used is a model equipped to handle dry feed and having a solid conveying zone, one or multiple heating zones, and an extrusion die. In some embodiments, the extruder may possess multiple separate temperature controllable heating zones. In some embodiments, the extruder used is a twin screw extruder or a conical extruder. In some embodiments, the extruder used is a twin screw extruder, for example, Leistritz ZSE 18 HPe.

Many conditions may be varied during the extrusion process to arrive at the pharmaceutical compositions described herein. Such conditions include, by way of example, formulation composition, feed rate, operating temperature, extruder screw RPM, residence time, die configuration, heating zone length and extruder torque and/or pressure. In view of the present description, methods for the optimization of such conditions will be known to the skilled artisan.

In some embodiments, the methods of making the pharmaceutical compositions described herein further comprise pelletization of an extruded composition comprising the TAF drug substance and the biodegradable polymer to obtain pellets comprising TAF drug substance and the biodegradable polymer. In some embodiments, a Bay Plastics BT 25 Lab Series Pelletizer is used.

In some embodiments, the extruded composition comprising the TAF drug substance and the biodegradable polymer is pelletized to obtain pellets of size (e.g. diameter and/or length) about 0.1 mm to about 30 mm.

In some embodiments, the extruded composition comprising TAF drug substance and the biodegradable polymer is pelletized to obtain pellets of size (e.g. diameter) about 0.1-25 mm, 0.1-20 mm, 0.1-15 mm, 0.1-10 mm, 0.1-5 mm, 0.1-4 mm, 0.1-3 mm, 0.1-2 mm, 0.1-1 mm, 0.1-0.5 mm, 0.5-30 mm, 0.5-25 mm, 0.5-20 mm, 0.5-15 mm, 0.5-10 mm, 0.5-5 mm, 0.5-4 mm, 0.5-3 mm, 0.5-2 mm, 0.5-1 mm, 1-30 mm, 1-25 mm, 1-20 mm, 1-15 mm, 1-10 mm, 1-5 mm, 1-4 mm, 1-3 mm, 1-2 mm, 5-30 mm, 5-25 mm, 5-20 mm, 5-15 mm, 5-10 mm, 10-30 mm, 10-25 mm, 10-20 mm, 10-15 mm, 15-30 mm, 15-25 mm, 15-20 mm, 20-30 mm, 20-25 mm, 03 25-30 mm. In some embodiments, the extruded composition comprising the TAF drug substance and the biodegradable polymer is pelletized to obtain pellets of size (e.g. diameter) about 0.1 mm to 10 mm. In some embodiments, the extruded composition comprising the TAF drug substance and the biodegradable polymer is pelletized to obtain pellets of size (e.g. diameter) about 0.5 mm to 5 mm. In some embodiments, the extruded composition comprising TAF drug substance and the biodegradable polymer is pelletized to obtain pellets of size (e.g. diameter) about 1 mm to 3 mm. In some embodiments, the extruded composition comprising TAF drug substance and the biodegradable polymer is pelletized to obtain pellets of size (e.g. diameter) about 0.5 mm to 3 mm.

In some embodiments, the extruded composition comprising TAF drug substance and the biodegradable polymer is pelletized to obtain pellets. Pellets may be substantially cylindrical. In some embodiments, the pellets are of size (e.g. length) about 0.1-25 mm, 0.1-20 mm, 0.1-15 mm, 0.1-10 mm, 0.1-5 mm, 0.1-4 mm, 0.1-3 mm, 0.1-2 mm, 0.1-1 mm, 0.1-0.5 mm, 0.5-30 mm, 0.5-25 mm, 0.5-20 mm, 0.5-15 mm, 0.5-10 mm, 0.5-5 mm, 0.5-4 mm, 0.5-3 mm, 0.5-2 mm, 0.5-1 mm, 1-30 mm, 1-25 mm, 1-20 mm, 1-15 mm, 1-10 mm, 1-5 mm, 1-4 mm, 1-3 mm, 1-2 mm, 5-30 mm, 5-25 mm, 5-20 mm, 5-15 mm, 5-10 mm, 10-30 mm, 10-25 mm, 10-20 mm, 10-15 mm, 15-30 mm, 15-25 mm, 15-20 mm, 20-30 mm, 20-25 mm, or 25-30 mm. In some embodiments, the extruded composition comprising the TAF drug substance and the biodegradable polymer is pelletized to obtain pellets of size (e.g. length) about 0.1 mm to 10 mm. In some embodiments, the extruded composition comprising the TAF drug substance and the biodegradable polymer is pelletized to obtain pellets of size (e.g. length) about 0.5 mm to 5 mm.

In some embodiments, the extruded composition comprising TAF drug substance and the biodegradable polymer is pelletized to obtain pellets of diameter 0.5-3.0 mm and length 0.5-5 mm.

In some embodiments, the methods of making the pharmaceutical compositions described herein further comprise cryomilling the pellets comprising the TAF drug substance and the biodegradable polymer to obtain particles comprising the TAF drug substance and the biodegradable polymer. In some embodiments, the methods further comprising classifying the particles (e.g. by particle size) comprising the TAF drug substance and the biodegradable polymer to obtain the pharmaceutical composition.

The percentage yield of the hot melt extrusion methods described herein is generally greater than 40%, wherein the percentage yield is =(weight of the pharmaceutical composition obtained/total weight of the TAF drug substance, the biodegradable polymer and/or additional agent)×100. In some embodiments, the percentage yield is greater than about 45%, about 50%, about 60%, about 70%, about 80%, about 90%, or about 95%. In some embodiments, the percentage yield is from about 45% to about 85%.

### Microspheres

In other embodiments, the methods of making the pharmaceutical compositions described herein comprise preparing microspheres comprising the TAF drug substance and the biodegradable polymer. In some embodiments, the methods comprise (i) mixing the TAF drug substance and the biodegradable polymer (e.g. PLGA) in a suitable solvent and (ii) adding an aqueous solution to the mixture of the TAF drug substance and the biodegradable polymer (e.g. PLGA) in the solvent. In some embodiments, the solvent is an organic solvent, for e.g. dichloromethane, ethanol, or a mixture thereof.

In some embodiments, the methods further comprise, removal of the solvent from the solvent/aqueous solution mixture. Removal of the solvent can be done by any suitable means for e.g. by heating and/or under reduced pressure. The thus obtained microspheres can be collected by any suitable means, for e.g. by centrifugation or filtration. The microspheres are optionally washed with water to remove free TAF drug substance, biodegradable polymer and the like adhered to the surface of the microspheres. The microspheres are then dried, for example under a reduced pressure, to remove a suitable amount of water and solvent in the microspheres. Optionally, the microspheres are freeze dried. Further, the thus obtained microspheres can be grounded, milled, and/or classified to obtain the desired size microspheres and to remove oversized microspheres.

In some embodiments, the microspheres further comprise additional agents. In some embodiments, the additional agent is a deoxycholate salt, for example sodium deoxycholate. In some embodiments, the additional agent is a therapeutic agent. In some embodiments, the additional agent is an anti-inflammatory agent. In some embodiments, the anti-inflammatory agent is a steroid, for example a corticosteroid. In some embodiments, the anti-inflammatory agent is dexamethasone. In some embodiments, the additional agent is a glucocorticoid. In some examples, the additional agent is methylprednisolone or methylprednisolone acetate (e.g. Depo-Medrol®).

### Spray Dried Dispersions

In some embodiments, the methods of making the pharmaceutical compositions described herein comprise obtaining spray dried compositions comprising the TAF drug substance and the biodegradable polymer. In some embodiments, the methods comprising mixing the TAF drug substance and the biodegradable polymer (e.g. PLGA) in a suitable solvent and spray drying the mixture. Any suitable solvent may be used. In some embodiments, the solvent is an organic solvent, for e.g. dichloromethane, acetone, ethyl acetate, or a mixture thereof. In some embodiments, the methods may comprise further drying (secondary drying) the spray dried compositions (also referred to as the primary dried composition). In some embodiments, the secondary drying comprises drying under vacuum and/or heating.

In some embodiments, the spray dried compositions further comprises additional agents. In some embodiments, the additional agent is glycerol monostearate. In some embodiments, the additional agent is cholesterol. In some embodiments, the additional agent is a therapeutic agent. In some embodiments, the additional agent is an anti-inflammatory agent. In some embodiments, the anti-inflammatory agent is a steroid, for example a corticosteroid. In some embodiments, the anti-inflammatory agent is dexamethasone. In some embodiments, the additional agent is a glucocorticoid. In some examples, the additional agent is methylprednisolone or methylprednisolone acetate (e.g. Depo-Medrol®).

### Pharmaceutical Compositions

The pharmaceutical compositions obtained by the methods described herein (hot melt extrusion, spray dried dispersions, and microspheres) may optionally be further processed and/or classified to obtain the desired particle size. In some embodiments, the pharmaceutical composition is a micronized composition. In some embodiments, the pharmaceutical compositions described herein have a d₉₀ value of about 50 μm-400 μm. In some embodiments, the d₉₀ value is about 50-350 μm, 50-300 μm, 50-250 μm, 50-200 μm, 50-150 μm, 50-100 μm, 100-400 μm, 100-350 μm, 100-300 μm, 100-250 μm, 100-200 μm, 100-150 μm, 100-400 μm, 150-350 μm, 150-300 μm, 150-250 μm, 150-200 μm, 200-400 μm, 200-350 μm, 200-300 μm, 200-250 μm, 250-400 μm, 250-350 μm, 250-300 μm, 300-400 μm, 300-350 μm, or 350-400 μm. In some embodiments, the pharmaceutical compositions described herein have a d₉₀ value of about 100 μm-250 μm. In some embodiments, the pharmaceutical compositions described herein have a d₉₀ value of about 100 μm-150 μm.

In some embodiments, the pharmaceutical compositions described herein have a d₉₀ value of less than about 400 μm, about 350 μm, about 300 μm, about 250 μm, about 200 μm, about 150 μm, about 100 μm, or about 50 μm. In some embodiments, the pharmaceutical compositions have a d₉₀ value of less than about 250 μm. In some embodiments, the pharmaceutical compositions have a d₉₀ value of less than about 150 μm. In some embodiments, the pharmaceutical compositions have a d₉₀ value of about 140 μm, about 139 μm, about 138 μm, about 137 μm, about 136 μm, about 135 μm, about 134 μm, about 133 μm, about 132 μm, about 131 μm, about 130 μm, about 129 μm, about 128 μm, about 127 μm, about 126 μm, about 125 μm, about 124 μm, about 123 μm, about 122 μm, about 121 μm, about 120 μm, about 119 μm, about 118 μm, about 117 μm, about 116 μm or about 115 μm. In some embodiments, the composition has a d₉₀ value of about 132 μm.

In some embodiments, the composition has a d₉₀ value of about 120-200 μm. In some embodiments, the composition has a d₉₀ value of about 130-160 μm. In some embodiments, the composition has a d₉₀ value of about 150-160 μm. In some embodiments, the composition has a d₉₀ value of about 151 μm, 152 μm, 153 μm, 154 μm, 155 μm, 156 μm, 157 μm, 158 μm, 159 μm, or 160 μm.

In some embodiments, the pharmaceutical compositions described herein have a d₅₀ value of about 30-150 μm. In some embodiments, the pharmaceutical composition has a d₅₀ value of about 30-140 μm, 30-130 μm, 30-120 μm, 30-110 μm, 30-100 μm, 30-90 μm, 30-80 μm, 30-70 μm, 30-60 μm, 30-50 μm, 30-40 μm, 40-150 μm, 40-140 μm, 40-130 μm, 40-120 μm, 40-110 μm, 40-100 μm, 40-90 μm, 40-80 μm, 40-70 μm, 40-60 μm, 40-50 μm, 50-150 μm, 50-140 μm, 50-130 μm, 50-120 μm, 50-110 μm, 50-100 μm, 50-90 μm, 50-80 μm, 50-70 μm, 50-60 μm, 60-150 μm, 60-140 μm, 60-130 μm, 60-120 μm, 60-110 μm, 60-100 μm, 60-90 μm, 60-80 μm, 60-70 μm, 70-150 μm, 70-140 μm, 70-130 μm, 70-120 μm, 70-110 μm, 70-100 μm, 70-90 μm, 70-80 μm, 80-150 μm, 80-140 μm, 80-130 μm, 80-120 μm, 80-110 μm, 80-100 μm, 80-90 μm, 90-150 μm, 90-140 μm, 90-130 μm, 90-120 μm, 90-110 μm, 90-100 μm, 100-150 μm, 100-140 μm, 100-130 μm, 100-120 μm, 100-110 μm, 110-150 μm, 110-140 μm, 110-130 μm, 110-120 μm, 120-150 μm, 120-140 μm, 120-130 μm, 130-150 μm, 130-140 μm, or 140-150 μm.

In some embodiments, the pharmaceutical composition described herein have a d₅₀ value of about a d₅₀ value of about 80-150 μm. In some embodiments, the pharmaceutical composition described herein have a d₅₀ value of about a d₅₀ value of about 80-110 μm. In some embodiments, the pharmaceutical composition described herein have a d₅₀ value of about a d₅₀ value of about 80-100 μm, for example about 98-82 μm, about 96-84 μm, about 94-86 μm, or about 92-88 μm. In some embodiments, the pharmaceutical composition has a d₅₀ value of about a d₅₀ value of about 90 μm.

In some embodiments, the pharmaceutical compositions described herein have a d₁₀ value of about 10-100 μm. In some embodiments, the pharmaceutical compositions have a d₁₀ value of about 10-90 μm, 10-80 μm, 10-70 μm, 10-60 μm, 10-50 μm, 10-40 μm, 10-30 μm, 10-20 μm, 20-100 μm, 20-90 μm, 20-80 μm, 20-70 μm, 20-60 μm, 20-50 μm, 20-40 μm, 20-30 μm, 30-100 μm, 30-90 μm, 30-80 μm, 30-70 μm, 30-60 μm, 30-50 μm, 30-40 μm, 40-100 μm, 40-90 μm, 40-80 μm, 40-70 μm, 40-60 μm, 40-50 μm, 50-100 μm, 50-90 μm, 50-80 μm, 50-70 μm, 50-60 μm, 60-100 μm, 60-90 μm, 60-80 μm, 60-70 μm, 70-100 μm, 70-90 μm, 70-80 μm, 80-100 μm, 80-90 μm, or 90-100 μm. In some embodiments, the pharmaceutical compositions described herein have a d₁₀ value of about 35-80 μm.

In some embodiments, the pharmaceutical compositions described herein have a d₉₀ value of about 120-140 μm, a d₅₀ value of about 80-100 μm, and a d₁₀ value of about 50-70 μm. In some embodiments, the pharmaceutical composition described herein has a d₉₀ value of about 130-134 μm, a d₅₀ value of about 88-92 μm, and a d₁₀ value of about 58-62 μm. In some embodiments, the composition described herein has a d₉₀ value of about 132 μm, a d₅₀ value of about 90 μm, and a d₁₀ value of about 60 μm.

In some embodiments, the pharmaceutical compositions further comprise an additional therapeutic agent. In some embodiments, the additional agent is an anti-inflammatory agent. In some embodiments, the anti-inflammatory agent is a steroid, for example a corticosteroid. In some embodiments, the anti-inflammatory agent is dexamethasone. In some embodiments, the additional agent is a glucocorticoid. In some examples, the additional agent is methylprednisolone or methylprednisolone acetate (e.g. Depo-Medrol®).

In some embodiments, the pharmaceutical compositions do not comprise an additional therapeutic agent. In other words, the TAF drug substance is the sole therapeutic agent.

In some embodiments, the pharmaceutical compositions do not comprise an additional anti-HIV agent. In other words, the TAF drug substance is the sole anti-HIV agent. In particular, provided are pharmaceutical compositions comprising TAF or a pharmaceutically acceptable salt thereof, and an anti-inflammatory agent (such as a corticosteroid, more particularly dexamethasone) as sole active ingredients.

Provided is a pharmaceutical composition comprising TAF free base and a biodegradable polymer. Provided is a pharmaceutical composition comprising TAF free base and a biodegradable polymer, wherein the TAF free base and the biodegradable polymer together constitute 90-100%, e.g. 99-100%, of the composition weight. Provided are the above compositions wherein TAF free base is in a crystalline form, for example crystalline Form I of TAF free base.

Provided is a pharmaceutical composition comprising a compound of Formula I or a pharmaceutically acceptable salt thereof and PLGA. Provided is a pharmaceutical composition comprising a compound of Formula I or a pharmaceutically acceptable salt thereof and PLGA, wherein the compound of Formula I or a pharmaceutically acceptable salt thereof and PLGA together constitute 90-100%, e.g. 99-100%, of the composition weight. Provided are the above composition wherein PLGA is PLGA8515 (about 85% lactic acid and about 15% glycolic acid).

Provided is a pharmaceutical composition comprising about 15-35% w/w compound of Formula I or a pharmaceutically acceptable salt thereof and 55-85% w/w biodegradable polymer. Provided is a pharmaceutical composition comprising about 15-35% w/w TAF free base and 55-85% w/w biodegradable polymer. Provided is a pharmaceutical composition comprising about 15-35% w/w crystalline form of TAF free base and 55-85% w/w biodegradable polymer. Provided is a pharmaceutical composition comprising about 18-20% w/w crystalline form of TAF free base and 80-82% w/w PLGA. Provided is a pharmaceutical composition comprising about 19% w/w crystalline form of TAF free base and about 81% w/w PLGA. Provided is a pharmaceutical composition comprising about 19% w/w crystalline form of TAF free base and about 81% w/w PLGA8515 (about 85% lactic acid and about 15% glycolic acid). Provided are the above compositions prepared by hot melt extrusion.

Provided is a pharmaceutical composition comprising about 15-35% w/w crystalline Form I of TAF free base and 55-85% w/w biodegradable polymer. Provided is a pharmaceutical composition comprising about 18-20% w/w crystalline Form I of TAF free base and 80-82% w/w PLGA. Provided is a pharmaceutical composition comprising about 19% w/w crystalline Form I of TAF free base and about 81% w/w PLGA. Provided is a pharmaceutical composition comprising about 19% w/w crystalline Form I of TAF free base and about 810% w/w PLGA8515 (about 85% lactic acid and about 15% glycolic acid). Provided are the above compositions prepared by hot melt extrusion.

Provided is a pharmaceutical composition comprising about 18-20% w/w crystalline form of TAF free base and 80-82% w/w PLGA, where TAF free base is in micronized form. Provided is a pharmaceutical composition comprising about 18-20% w/w crystalline form of TAF free base and 80-82% w/w PLGA, where TAF free base is in micronized form and has a d₉₀ of about 1-10 μm, a d₅₀ of about 1-5 μm and a d₁₀ of about 0.1-2 μm. Provided is a pharmaceutical composition prepared by hot melt extrusion comprising about 18-20% w/w crystalline form of TAF free base and 80-82% w/w PLGA, where TAF free base is in micronized form, and wherein the composition is micronized. Provided is a pharmaceutical composition comprising about 18-20% w/w crystalline form of TAF free base and 80-82% w/w PLGA, where TAF free base is in micronized form, and wherein the composition is micronized having a d₉₀ value of about 120-140 μm, a d₅₀ value of about 80-100 μm and a d₁₀ value of about 35-80 μm. Provided are the above compositions prepared by hot melt extrusion.

### v. SUSPENDING VEHICLE

In various embodiments, the pharmaceutical compositions described herein are reconstituted before administration to the subject. In various embodiments, the pharmaceutical compositions described herein are reconstituted with a suspending vehicle to obtain a suspension for injection. In some embodiments, the pharmaceutical compositions described herein are reconstituted up to 30 mins prior to administration to the subject. In some embodiments, the pharmaceutical compositions described herein are reconstituted up to 25, 20, 15, 10, 5, 4, 3, 2, or 1 mins prior to administration to the subject. In some embodiments, the pharmaceutical compositions described herein are reconstituted less than 25, 20, 15, 10, 5, 4, 3, 2, or 1 mins prior to administration to the subject. In some embodiments, the pharmaceutical compositions described herein are reconstituted immediately before administration to the subject.

In various embodiments, the suspending vehicle comprise one or more agents selected from the group consisting of water for injection, a suspending agent, a wetting agent, and/or a buffer.

In some embodiments, the suspending agent is selected from the group consisting of methyl cellulose, carboxy methyl cellulose, hydroxypropyl methylcellulose, and povidone. In some embodiments, the suspending agent is selected from the group consisting of carboxy methyl cellulose, hydroxypropyl methylcellulose, and povidone. In some embodiments, the suspending agent is carboxy methyl cellulose or povidone. In some embodiments, the suspending agent comprises carboxy methyl cellulose and povidone.

In some embodiments, the suspending agent is selected from the group consisting of methyl cellulose, carboxy methyl cellulose, hydroxypropyl methylcellulose, and povidone K12, povidone K17, povidone K25, Povidone K30, or Povidone K90. In some embodiments, the suspending agent is selected from the group consisting of carboxy methyl cellulose, hydroxypropyl methylcellulose, and povidone K12, povidone K17, povidone K25, Povidone K30, or Povidone K90. In some embodiments, the suspending agent is carboxy methyl cellulose or povidone K12. In some embodiments, the suspending agent comprises carboxy methyl cellulose and povidone K12.

The amount of carboxy methyl cellulose in the suspending vehicle may vary. In some embodiments, the amount of carboxy methyl cellulose in the suspending vehicle is from about 0.1% to about 5.0% w/w. For example, the amount of carboxy methyl cellulose is about 0.1%-4.5%, 0.1%-4.0%, 0.1%-3.5%, 0.1%-3.0%, 0.1%-2.5%, 0.1%-2.0%, 0.1%-1.5%, 0.1%-1.0%, 0.5%-5.0%, 0.5%-4.5%, 0.5%-4.0%, 0.5%-3.5%, 0.5%-3.0%, 0.5%-2.5%, 0.5%-2.0%, or 0.5%-1.5% w/w. In some embodiments, the amount of carboxy methyl cellulose in the suspending vehicle is from about 0.5% to about 2.0% w/w. In some embodiments, the amount of carboxy methyl cellulose in the suspending vehicle is about 1.0% w/w.

The amount of povidone K12 in the suspending vehicle may vary. In some embodiments, the amount of povidone K12 in the suspending vehicle is from about 0.1% to about 5.0% w/w. For example, the amount of povidone K12 is about 0.1%-4.5%, 0.1%-4.0%, 0.1%-3.5%, 0.1%-3.0%, 0.1%-2.5%, 0.1%-2.0%, 0.1%-1.5%, 0.1%-1.0%, 0.5%-5.0%, 0.5%-4.5%, 0.5%-4.0%, 0.5%-3.5%, 0.5%-3.0%, 0.5%-2.5%, 0.5%-2.0%, 0.5%-1.5% w/w. In some embodiments, the amount of povidone K12 in the suspending vehicle is from about 0.5% to about 2.0% w/w. In some embodiments, the amount of povidone K12 in the suspending vehicle is from about 1.0% w/w.

In some embodiments, the wetting agent is selected from a group consisting of a polysorbate, a poloxamer, Lecithin, a fatty acid polyethylene glycol ester, an ethoxylated castor oil, sorbitane trioleate, and sodium deoxycholate. In some embodiments, the wetting agent is selected from a group consisting of a polysorbate, a poloxamer, Lecithin, polyethylene glycol (15)-hydroxystearate, polyoxyl 35 hydrogenated castor oil, sorbitane trioleate, and sodium deoxycholate

In some embodiments, the wetting agent is selected from a group consisting of a polysorbate, poloxamer, Lecithin, solutol, cremophor EL, span, and sodium deoxycholate.

In some embodiments, the wetting agent is selected from the group consisting of Tween 20, Tween 80, poloxamer 188, poloxamer 338, poloxamer 407, poloxamer 213, poloxamer 2930, lecithin, solutol HS-15, cremophor EL, span 85, and sodium deoxycholate.

In some embodiments, the wetting agent is selected from the group consisting of Tween 80, poloxamer 188, lecithin, solutol HS-15, cremophor EL, span 85, and sodium deoxycholate.

In some embodiments, the wetting agent is Tween. In some embodiments, the wetting agent is Tween 20, Tween 40, Tween 60, Tween 65, or Tween 80. In some embodiments, the wetting agent is Tween 80.

The amount of wetting agent (e.g. Tween 80) in the suspending vehicles may vary. In some embodiments, the amount of wetting agent in the suspending vehicle is from about 0.02% to about 2.0% w/w. In some embodiments, the amount of wetting agent in the suspending vehicle is from about 0.02%-1.50%, 0.02%-1.00%, 0.02%-0.50%, 0.02%-0.25%, 0.05%-2.00%, 0.05%-1.50%, 0.05%-1.00%, 0.05%-0.50%, 0.05%-0.25%, 0.10%-2.00%, 0.10%-1.50%, 0.10%-1.00%, 0.10%-0.50%, 0.10%-0.25%. In some embodiments, the amount of wetting agent (e.g. Tween 80) in the suspending vehicle is from about 0.1%-0.5% w/w. In some embodiments, the amount of wetting agent (e.g. Tween 80) in the suspending vehicle is from about 0.1%-0.3% w/w. In some embodiments, the amount of wetting agent (e.g. Tween 80) in the suspending vehicle is from about 0.2% w/w.

In various embodiments, the suspending vehicles described herein further comprise a buffering agent. In some embodiments, the buffer is a phosphate buffer. In some embodiments, the buffer comprises sodium phosphate monobasic, sodium phosphate dibasic, or sodium chloride. In some embodiments, the buffer is a phosphate buffered saline of pH 7.4. In some embodiments, the amount of the PBS buffer is about 95%-99%, for example 95%-98%, 96%-99%, 96%-98%, 97-99%, or 97-98% w/w. In some embodiments, the amount of the PBS buffer is about 97%-98% w/w. In some embodiments, the amount of the PBS buffer is about 97.8% w/w.

In some embodiments, the suspending vehicle comprises carboxymethyl cellulose, povidone K12, Tween 80, and PBS (pH 7.4). In some embodiments, the suspending vehicle comprises carboxymethyl cellulose (0.5%-2.0% w/w), povidone K12 (0.5%-2.0% w/w), Tween 80 (0.1%-0.3% w/w), and PBS (pH 7.4) (97%-98% w/w). In some embodiments, the suspending vehicle comprises carboxymethyl cellulose (1% w/w), povidone K12 (1% w/w), Tween 80 (0.2% w/w), and PBS (pH 7.4) (97.8% w/w).

Substances with a trade name may be purchased from commercial sources. Solutol HS-15 is polyethylene glycol (15)-hydroxystearate. Cremophor EL is polyoxyl 35 hydrogenated castor oil. Tween 20 is polysorbate 20. Tween 40 is polysorbate 40, Tween 60 is polysorbate 60, Tween 65 is polysorbate 65, Tween 80 is polysorbate 80. Span 85 is sorbitane trioleate. It is understood that for each embodiment herein involving a substance characterised by a trade name, a corresponding embodiment is provided where the generic name is used instead.

### vi. SUSPENSIONS FOR INJECTION

In various embodiments, the pharmaceutical compositions described herein are reconstituted with the suspending vehicles described herein to obtain suspensions for injection. Thus, provided is a suspension suitable for injection comprising TAF drug substance, a biodegradable polymer and a suspending vehicle.

Also provided is a method for making a pharmaceutical formulation suitable for injection comprising the step of mixing a pharmaceutical composition provided herein with a suspending vehicle provided herein.

In some embodiments, the amount of the TAF drug substance in the suspensions for injection is about 40-60 mg/mL. In some embodiments, the amount of the TAF drug substance in the suspension for injection is about 40-55 mg/mL, about 40-50 mg/mL, about 40-45 mg/mL, about 45-55 mg/mL, about 45-50 mg/mL, or about 50-55 mg/mL. In some embodiments, the amount of TAF drug substance in the suspension for injection is about 45-50 mg/mL, for example about 45 mg/mL, about 46 mg/mL, about 47 mg/mL, about 48 mg/mL, about 49 mg/mL, about 50 mg/mL. In some embodiments, the amount of the TAF drug substance in the suspension for injection is about 48 mg/mL.

The volume of suspension for injection administered per administration (i.e. one dose of the suspension for injection) is about 0.1-5 mL. In some embodiments, the volume of the suspension for injection administered per administration is about 0.1-4.5 mL, about 0.1-4.0 mL, about 0.1-3.5 mL, about 0.1-3.0 mL, about 0.1-2.5 mL, about 0.1-2.0 mL, about 0.1-1.5 mL, about 0.1-1.0 mL, about 0.1-0.5 mL, about 0.5-5.0 mL, about 0.5-4.5 mL, about 0.5-4.0 mL, about 0.5-3.5 mL, about 0.5-3.0 mL, about 0.5-2.5 mL, about 0.5-2.0 mL, about 0.5-1.5 mL, about 0.5-1.0 mL, about 1.0-5.0 mL, about 1-4.5 mL, about 1-4.0 mL, about 1-3.5 mL, about 1-3.0 mL, about 1-2.5 mL, about 1-2.0 mL, about 1-1.5 mL, about 1.5-5.0 mL, about 1.5-4.5 mL, about 1.5-4.0 mL, about 1.5-3.5 mL, about 1.5-3.0 mL, about 1.5-2.5 mL, about 1.5-2.0 mL, about 2.0-5.0 mL, about 2.0-4.5 mL, about 2.0-4.0 mL, about 2.0-3.5 mL, about 2.0-3.0 mL, about 2.0-2.5 mL, about 2.5-5.0 mL, about 2.5-4.5 mL, about 2.5-4.0 mL, about 2.5-3.5 mL, about 2.5-3.0 mL, about 3.0-5.0 mL, about 3.0-4.5 mL, about 3.0-4.0 mL, about 3.0-3.5 mL, about 3.5-5.0 mL, about 3.5-4.5 mL, about 3.5-4.0 mL, about 4.0-5.0 mL, about 4.0-4.5 mL, or about 4.5-5.0 mL.

In some embodiments, the volume of the suspension for injection administered is about 1.5-3.5 mL, for example about 1.5 mL, about 1.6 mL, about 1.7 mL, about 1.8 mL, about 1.9 mL, about 2.0 mL, about 2.1 mL, about 2.2 mL, about 2.3 mL, about 2.4 mL, about 2.5 mL, about 2.6 mL, about 2.7 mL, about 2.8 mL, about 2.9 mL, about 3.0 mL, about 3.1 mL, about 3.2 mL, about 3.3 mL, about 3.4 mL, or about 3.5 mL.

In some embodiments, the volume of the suspension for injection administered is about 1.8-2.2 mL. In some embodiments, the volume of the suspension administered is about 2.0 mL. In some embodiments, the volume of the suspension administered is about 2.1 mL.

The suspension for injection described herein can be administered by any suitable methods. In some embodiments, the suspension for injection described herein are administered subcutaneously. In some embodiments, the suspension for injection described herein are administered by injection.

The suspensions for injection described herein are long acting formulations of the TAF drug substance, which are administered once every 1 week to about 6 months. In some embodiments, the suspension for injection described herein are administered at a frequency of about 1 week to about 5 months, about 1 week to about 4 months, about 1 week to about 3 months, about 1 week to about 2 months, about 1 week to about 1 month, about 1 week to about 4 weeks, about 1 week to about 3 weeks, about 1 week to about 2 weeks, about 2 weeks to 6 months, about 2 weeks to about 5 months, about 2 weeks to about 4 months, about 2 weeks to about 3 months, about 2 weeks to about 2 months, about 2 weeks to about 1 month, about 2 weeks to about 4 weeks, about 2 weeks to about 3 weeks, about 3 weeks to about 6 months, about 3 weeks to about 5 months, about 3 weeks to about 4 months, about 3 weeks to about 3 months, about 3 weeks to about 2 months, about 3 weeks to about 1 month, about 3 weeks to about 4 weeks, about 4 weeks to about 6 months, about 4 weeks to about 5 months, about 4 weeks to about 4 months, about 4 weeks to about 3 months, about 4 weeks to about 2 months, about 4 weeks to about 1 month, about 1 month to about 6 months, about 1 month to about 5 months, about 1 month to about 4 months, about 1 month to about 3 months, about 1 month to about 3 months, about 2 months to about 6 months, about 2 months to about 5 months, about 2 months to about 4 months, about 2 months to about 3 months, about 2 months to about 3 months, about 3 months to about 6 months, about 3 months to about 5 months, about 3 months to about 4 months, about 4 months to about 6 months, about 3 months to about 5 months, or about 5 months to about 6 months.

In some embodiments, the suspensions for injection described herein are administered once every 28 days. In some embodiments, the suspensions for injection described herein are administered once every 1 month. In some embodiments, the suspensions for injection described herein are administered at a frequency of once every month or less. In some embodiments, the suspensions for injection described herein are administered at a frequency of once every two months or less. In some embodiments, the suspensions for injection described herein are administered at a frequency of once every three months or less.

### vii. COMPOSITIONS AND KITS

The pharmaceutical compositions and/or the suspending vehicles provided herein can be comprised in a kit. In one aspect, provided herein are kits comprising a pharmaceutical composition described herein in a suitable packaging. In some embodiments, the kits further comprises instructions for using the pharmaceutical compositions.

In some embodiments, the kits comprise a pharmaceutical composition provided herein and a suspending vehicle provided herein. In some embodiments, the kits comprise a pharmaceutical composition provided herein in a first container and a suspending vehicle provided herein in a second container. In some embodiments, the pharmaceutical composition is comprised in a glass/plastic vial with a cap seal. In some embodiments, the suspending vehicle is comprised in a glass/plastic vial with a cap seal. In some embodiments, the pharmaceutical composition and the suspending vehicle are each comprised in a different glass/plastic vial with a cap seal.

In some embodiments, the kits described herein further comprise instructions for reconstituting the pharmaceutical composition with the suspending vehicle to obtain the suspension for injection. In some embodiments, the kit further provides instructions for using the suspension for injection.

In some embodiments, the kits provided herein further comprise a device for administration of the suspension for injection to a subject. In some embodiments, the device is an injection device. In some embodiments, the device comprises a syringe and a needle. In some embodiments, the device comprises a disposable syringe and a needle. In some embodiments, the needle is a 19 gauge or a 20 gauge needle.

In some embodiments, described herein are kits wherein the pharmaceutical composition is comprised in a glass or a plastic vial with a cap seal or a vial adaptor and the suspending vehicle is comprised in a syringe. In some embodiments, the pharmaceutical composition is comprised in a plastic vial with a vial adaptor and the suspending vehicle is comprised in a syringe. In some embodiments, the kit further comprises a needle. In some embodiments, the needle is a 19 gauge or a 20 gauge needle.

In some embodiments, provided herein are kits comprising a dual chamber syringe, wherein one chamber of the dual chamber syringe comprises the pharmaceutical composition and the other chamber of the dual chamber syringe comprises the suspending vehicle. The kits may further comprise a needle, for examples a 19 gauge or a 20 gauge needled.

In some embodiments, the kits provided herein comprise the pharmaceutical composition, the suspending vehicle, and a wearable injection device.

In various embodiments, the kits described herein further comprise one or more (e.g., one, two, three, four, one or two, one to three, or one to four) additional therapeutic agents, or a pharmaceutically acceptable salt thereof.

In some embodiments, the kits provided herein further comprise an additional therapeutic agent, wherein the additional agent is an anti-inflammatory agent. In some embodiments, the anti-inflammatory agent is a steroid, for example a corticosteroid. In some embodiments, the anti-inflammatory agent is dexamethasone. In some embodiments, the additional agent is a glucocorticoid. In some examples, the additional agent is methylprednisolone or methylprednisolone acetate (e.g. Depo-Medrol®).

### viii. METHOD OF TREATMENT

Also provided herein are methods for the prevention or treatment of a disease in a subject in need thereof, wherein the methods comprise administering to the subject the pharmaceutical compositions described herein. In some embodiments, the pharmaceutical compositions described herein are administered after reconstitution with a suspending vehicle described herein.

In some embodiments provided herein are methods for treating a human immunodeficiency virus (HIV) infection in a subject in need thereof, wherein the methods comprise administering to the subject in need thereof a therapeutically effective amount of the pharmaceutical compositions described herein. In some embodiments, the pharmaceutical compositions described herein are administered after reconstitution with a suspending vehicle described herein.

In some embodiments, the methods for treating the HIV infections further comprises administering to the subject one or more additional therapeutic agents selected from the group consisting of HIV protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

In some embodiments, provided herein are methods of treating an HIV (e.g., HIV-1 and/or HIV-2) infection in a human having or at risk of having the HIV infection, wherein the methods comprise administering to the human a therapeutically effective amount of the pharmaceutical composition described herein. In some embodiments, the methods further comprise administering to the human a therapeutically effective amount of one, two, three, or four additional therapeutic agents. In certain embodiments, the additional therapeutic agent or agents are anti-HIV agents. In particular embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs (broadly neutralizing HIV antibodies), TLR7 agonists, pharmacokinetic enhancers, other drugs for treating HIV, or combinations thereof. In one embodiment, the additional therapeutic agent or agents are abacavir, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.

In some embodiments, the methods described herein further comprise administering to the human an anti-inflammatory agent to eliminate, reduce or prevent injection site reaction (ISR). In some embodiments, the anti-inflammatory agent is a steroid, for example a corticosteroid. In some embodiments, the anti-inflammatory agent is dexamethasone. In some embodiments, the additional agent is a glucocorticoid. In some examples, the additional agent is methylprednisolone or methylprednisolone acetate (e.g. Depo-Medrol®).

In another embodiment, the description provides the use of the compositions described herein in medical therapy. For example, the compositions described herein are used for treating an HIV (e.g., HIV-1 and/or HIV-2) infection in a human having or at risk of having the infection.

In some embodiments, provided herein are methods of treating the proliferation of the HIV virus, treating AIDS, or delaying the onset of AIDS or ARC symptoms in a mammal (e.g., a human), wherein the methods comprise administering to the mammal a pharmaceutical composition described herein.

In some embodiments, the pharmaceutical compositions described herein are used in preventing HIV infection. For example, in some embodiments, the pharmaceutical composition described herein are for use in pre-exposure prophylaxis (PrEP), i.e., before the exposure of the individual to the HIV virus to prevent HIV infection from taking hold if the individual is exposed to the virus and/or to keep the virus from establishing a permanent infection and/or to prevent the appearance of symptoms of the disease and/or to prevent the virus from reaching detectable levels in the blood.

In some embodiments, provided herein are the use of the pharmaceutical compositions described herein are for manufacture of a medicament for treating an HIV infection in a human being having or at risk of having the infection is described.

In another embodiment, an article of manufacture comprising a composition effective to treat an HIV infection; and packaging material comprising a label which indicates that the composition can be used to treat infection by HIV is described. Exemplary compositions comprise the pharmaceutical compositions described herein.

In some embodiments, described herein are methods of inhibiting the replication of HIV, the methods comprising exposing the virus to an effective amount of the pharmaceutical compositions described herein.

In some embodiments, provided herein are the use of the pharmaceutical compositions described herein to inhibit the replication of HIV.

In some embodiments, provided herein are methods for treating a hepatitis B virus (HBV) infection in a subject in need thereof, wherein the methods comprise administering to the subject in need thereof a therapeutically effective amount of the long acting formulations described herein.

Provided is TAF or a pharmaceutically acceptable salt thereof for use in a method of medical therapy, such as treating or preventing HIV, wherein the TAF is administered in a composition or formulation described herein.

Provided is TAF or a pharmaceutically acceptable salt thereof for use in a method of treating or preventing HIV, wherein the TAF is administered with dexamethasone. Provided is TAF or a pharmaceutically acceptable salt and dexamethasone for use in a method of medical therapy, such as treating or preventing HIV.

Provided is TAF or a pharmaceutically acceptable salt thereof for use in a method of treating or preventing HIV, wherein TAF or a pharmaceutically acceptable salt thereof is subcutaneously administered. Provided is TAF or a pharmaceutically acceptable salt thereof for use in a method of treating or preventing HIV, wherein TAF or a pharmaceutically acceptable salt thereof is subcutaneously administered once monthly.

### ix. COMBINATION THERAPY

### HBV Combination Therapy

In certain embodiments, a method for treating or preventing an HBV infection in a human having or at risk of having the infection is provided, comprising administering to the human a therapeutically effective amount of a composition described herein, in combination with a therapeutically effective amount of one or more (e.g., one, two, three, four, one or two, one to three, or one to four) additional therapeutic agents. In one embodiment, a method for treating an HBV infection in a human having or at risk of having the infection is provided, comprising administering to the human a therapeutically effective amount of a composition described herein, in combination with a therapeutically effective amount of one or more (e.g., one, two, three, four, one or two, one to three, or one to four) additional therapeutic agents.

In certain embodiments, the present description provides a method for treating an HBV infection, comprising administering to a patient in need thereof a therapeutically effective amount of a composition described herein, in combination with a therapeutically effective amount of one or more (e.g., one, two, three, four, one or two, one to three, or one to four) additional therapeutic agents which are suitable for treating an HBV infection.

The compounds described herein may be used or combined with one or more of a chemotherapeutic agent, an immunomodulator, an immunotherapeutic agent, a therapeutic antibody, a therapeutic vaccine, a bispecific antibody and “antibody-like” therapeutic protein (such as DARTs®, Duobodies®, Bites®, XmAbs®, TandAbs®, Fab derivatives), an antibody-drug conjugate (ADC), gene modifiers or gene editors (such as CRISPR Cas9, zinc finger nucleases, homing endonucleases, synthetic nucleases, TALENs), cell therapies such as CAR-T (chimeric antigen receptor T-cell), and TCR-T (an engineered T cell receptor) agent or any combination thereof.

### HBV Combination Drugs

Examples of combination drugs for the treatment of HBV include TRUVADA® (tenofovir disoproxil fumarate and emtricitabine); ABX-203, lamivudine, and PEG-IFN-alpha; ABX-203 adefovir, and PEG-IFNalpha; and INO-1800 (INO-9112 and RG7944).

Other HBV Drugs

Examples of other drugs for the treatment of HBV include alpha-hydroxytropolones, amdoxovir, beta-hydroxycytosine nucleosides, AL-034, CCC-0975, elvucitabine, ezetimibe, cyclosporin A, gentiopicrin (gentiopicroside), JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-0061A, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, UB-551, and ZH-2N, and the compounds described in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche).

HBV Vaccines

HBV vaccines include both prophylactic and therapeutic vaccines. Examples of HBV prophylactic vaccines include Vaxelis, Hexaxim, Heplisav, Mosquirix, DTwP-HBV vaccine, Bio-Hep-B, D/T/P/HBV/M (LBVP-0101; LBVW-0101), DTwP-Hepb-Hib-IPV vaccine, Heberpenta L, DTwP-HepB-Hib, V-419, CVI-HBV-001, Tetrabhay, hepatitis B prophylactic vaccine (Advax Super D), Hepatrol-07, GSK-223192A, ENGERIX B®, recombinant hepatitis B vaccine (intramuscular, Kangtai Biological Products), recombinant hepatitis B vaccine (Hansenual polymorpha yeast, intramuscular, Hualan Biological Engineering), recombinant hepatitis B surface antigen vaccine, Bimmugen, Euforavac, Eutravac, anrix-DTaP-IPV-Hep B, HBAI-20, Infanrix-DTaP-IPV-Hep B-Hib, Pentabio Vaksin DTP-HB-Hib, Comvac 4, Twinrix, Euvax-B, Tritanrix HB, Infanrix Hep B, Comvax, DTP-Hib-HBV vaccine, DTP-HBV vaccine, Yi Tai, Heberbiovac HB, Trivac HB, GerVax, DTwP-Hep B-Hib vaccine, Bilive, Hepavax-Gene, SUPERVAX, Comvac5, Shanvac-B, Hebsulin, Recombivax HB, Revac B mcf, Revac B+, Fendrix, DTwP-HepB-Hib, DNA-001, Shan5, Shan6, rhHBsAG vaccine, HBI pentavalent vaccine, LBVD, Infanrix HeXa, and DTaP-rHB-Hib vaccine.

Examples of HBV therapeutic vaccines include HBsAG-HBIG complex, ARB-1598, Bio-Hep-B, NASVAC, abi-HB (intravenous), ABX-203, Tetrabhay, GX-110E, GS-4774, peptide vaccine (epsilonPA-44), Hepatrol-07, NASVAC (NASTERAP), IMP-321, BEVAC, Revac B mcf, Revac B+, MGN-1333, KW-2, CVI-HBV-002, AltraHepB, VGX-6200, FP-02, FP-02.2, TG-1050, NU-500, HBVax, im/TriGrid/antigen vaccine, Mega-CD40L-adjuvanted vaccine, HepB-v, RG7944 (INO-1800), recombinant VLP-based therapeutic vaccine (HBV infection, VLP Biotech), AdTG-17909, AdTG-17910 AdTG-18202, ChronVac-B, TG-1050, and Lm HBV.

HBV DNA Polymerase Inhibitors

Examples of HBV DNA polymerase inhibitors include adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, SNC-019754, FMCA, AGX-1009, AR-II-04-26, HIP-1302, tenofovir disoproxil aspartate, tenofovir disoproxil orotate, and HS-10234.

Immunomodulators

Examples of immunomodulators include rintatolimod, imidol hydrochloride, ingaron, dermaVir, plaquenil (hydroxychloroquine), proleukin, hydroxyurea, mycophenolate mofetil (MPA) and its ester derivative mycophenolate mofetil (MMF), JNJ-440, WF-10, AB-452, ribavirin, IL-12, INO-9112, polymer polyethyleneimine (PEI), Gepon, VGV-1, MOR-22, CRV-431, JNJ-0535, TG-1050, ABI-H2158, BMS-936559, GS-9688, RO-7011785, RG-7854, AB-506, RO-6871765, AIC-649, and IR-103.

### Toll-Like Receptor (TLR) Modulators

TLR modulators include modulators of TLR1, TLR2, TLR3, TLR4, TLR5, TLR6, TLR7, TLR8, TLR9, TLR10, TLR11, TLR12, and TLR13. Examples of TLR3 modulators include rintatolimod, poly-ICLC, RIBOXXON®, Apoxxim, RIBOXXIM®, IPH-33, MCT-465, MCT-475, and ND-1.1.

Examples of TLR7 modulators include GS-9620 (vesatolimod), GSK-2245035, imiquimod, resiquimod, DSR-6434, DSP-3025, IMO-4200, MCT-465, MEDI-9197, 3M-051, SB-9922, 3M-052, Limtop, D, telratolimod, SP-0509, TMX-30X, TMX-202, RG-7863, RG-7795, LHC-165, RG-7854, and the compounds described in US20100143301 (Gilead Sciences), US20110098248 (Gilead Sciences), and US20090047249 (Gilead Sciences).

Examples of TLR8 modulators include motolimod, resiquimod, 3M-051, 3M-052, MCT-465, IMO-4200, VTX-763, VTX-1463, GS-9688 and the compounds described in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), US20130251673 (Novira Therapeutics), U.S. Pat. No. 9,670,205, US20160289229, U.S. patent application Ser. No. 15/692,161, and U.S. patent application Ser. No. 15/692,093.

Examples of TLR9 modulators include BB-001, BB-006, CYT-003, IMO-2055, IMO-2125, IMO-3100, IMO-8400, IR-103, IMO-9200, agatolimod, DIMS-9054, DV-1079, DV-1179, AZD-1419, leftolimod (MGN-1703), litenimod, and CYT-003-QbG10.

Examples of TLR7, TLR8 and TLR9 modulators include the compounds described in WO2017047769 (Teika Seiyaku), WO2015014815 (Janssen), WO2018045150 (Gilead Sciences Inc), WO2018045144 (Gilead Sciences Inc), WO2015162075 (Roche), WO2017034986 (University of Kansas), WO2018095426 (Jiangsu Hengrui Medicine Co Ltd), WO2016091698 (Roche), WO2016075661 (GlaxoSmithKline Biologicals), WO2016180743 (Roche), WO2018089695 (Dynavax Technologies), WO2016055553 (Roche), WO2015168279 (Novartis), WO2016107536 (Medshine Discovery), WO2018086593 (Livo (Shanghai) Pharmaceutical), WO2017106607 (Merck), WO2017061532 (Sumitomo Dainippon Pharma), WO2016023511 (Chia Tai Tianqing Pharmaceutical), WO2017076346 (Chia Tai Tianqing Pharmaceutical), WO2017046112 (Roche), WO2018078149 (Roche), WO2017040233 (3M Co), WO2016141092 (Gilead Sciences), WO2018049089 (Bristol Myers Squibb), WO2015057655 (Eisai Co Ltd), WO2017001307 (Roche), WO2018005586 (Bristol Myers Squibb), WO201704023 (3M Co), WO2017163264 (Council of Scientific and Industrial Research (India)), WO2018046460 (GlaxoSmithKline Biologicals), WO2018047081 (Novartis), WO2016142250 (Roche), WO2015168269 (Novartis), WO201804163 (Roche), WO2018038877 (3M Co), WO2015057659 (Eisai Co Ltd), WO2017202704 (Roche), WO2018026620 (Bristol Myers Squibb), WO2016029077 (Janus Biotherapeutics), WO201803143 (Merck), WO2016096778 (Roche), WO2017190669 (Shanghai De Novo Pharmatech), U.S. Ser. No. 09/884,866 (University of Minnesota), WO2017219931 (Sichuan KelunBiotech Biopharmaceutical), WO2018002319 (Janssen Sciences), WO2017216054 (Roche), WO2017202703 (Roche), WO2017184735 (IFM Therapeutics), WO2017184746 (IFM Therapeutics), WO2015088045 (Takeda Pharmaceutical), WO2017038909 (Takeda Pharmaceutical), WO2015095780 (University of Kansas), WO2015023958 (University of Kansas) Interferon Alpha Receptor Ligands

Examples of interferon alpha receptor ligands include interferon alpha-2b (INTRON A®), pegylated interferon alpha-2a (PEGASYS®), PEGylated interferon alpha-1b, interferon alpha 1b (HAPGEN®), Veldona, Infradure, Roferon-A, YPEG-interferon alfa-2a (YPEG-rhIFNalpha-2a), P-1101, Algeron, Alfarona, Ingaron (interferon gamma), rSIFN-co (recombinant super compound interferon), Ypeginterferon alfa-2b (YPEG-rhIFNalpha-2b), MOR-22, peginterferon alfa-2b (PEG-INTRON®), Bioferon, Novaferon, Inmutag (Inferon), MULTIFERON®, interferon alfa-n1 (HUMOFERON®), interferon beta-1a (AVONEX®), Shaferon, interferon alfa-2b (Axxo), Alfaferone, interferon alfa-2b (BioGeneric Pharma), interferon-alpha 2 (CJ), Laferonum, VIPEG, BLAUFERON-A, BLAUFERON-B, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), interferon alfa 2a, Optipeg A, Realfa 2B, Reliferon, interferon alfa-2b (Amega), interferon alfa-2b (Virchow), ropeginterferon alfa-2b, rHSA-IFN alpha-2a (recombinant human serum albumin intereferon alpha 2a fusion protein), rHSA-IFN alpha 2b, recombinant human interferon alpha-(1b, 2a, 2b), peginterferon alfa-2b (Amega), peginterferon alfa-2a, Reaferon-EC, Proquiferon, Uniferon, Urifron, interferon alfa-2b (Changchun Institute of Biological Products), Anterferon, Shanferon, Layfferon, Shang Sheng Lei Tai, INTEFEN, SINOGEN, Fukangtai, Pegstat, rHSA-IFN alpha-2b, SFR-9216, and Interapo (Interapa).

Hyaluronidase Inhibitors

Examples of hyaluronidase inhibitors include astodrimer.

Hepatitis B Surface Antigen (HBsAg) Inhibitors

Examples of HBsAg inhibitors include HBF-0259, PBHBV-001, PBHBV-2-15, PBHBV-2-1, REP-9AC, REP-9C, REP-9, REP-2139, REP-2139-Ca, REP-2165, REP-2055, REP-2163, REP-2165, REP-2053, REP-2031 and REP-006, and REP-9AC′.

Examples of HBsAg secretion inhibitors include BM601.

Cytotoxic T-lymphocyte-associated protein 4 (ipi4) inhibitors

Examples of Cytotoxic T-lymphocyte-associated protein 4 (ipi4) inhibitors include AGEN-2041, AGEN-1884, ipilumimab, belatacept, PSI-001, PRS-010, Probody mAbs, tremelimumab, and JHL-1155.

Cyclophilin Inhibitors

Examples of cyclophilin inhibitors include CPI-431-32, EDP-494, OCB-030, SCY-635, NVP-015, NVP-018, NVP-019, STG-175, and the compounds described in U.S. Pat. No. 8,513,184 (Gilead Sciences), US20140030221 (Gilead Sciences), US20130344030 (Gilead Sciences), and US20130344029 (Gilead Sciences).

HBV Viral Entry Inhibitors

Examples of HBV viral entry inhibitors include Myrcludex B.

Antisense Oligonucleotide Targeting Viral mRNA

Examples of antisense oligonucleotide targeting viral mRNA include ISIS-HBVRx, IONIS-HBVRx, IONIS-GSK6-LRx, GSK-3389404, RG-6004.

Short Interfering RNAs (siRNA) and ddRNAi

Examples of siRNA include TKM-HBV (TKM-HepB), ALN-HBV, SR-008, HepB-nRNA, and ARC-520, ARC-521, ARB-1740, ARB-1467.

Examples of DNA-directed RNA interference (ddRNAi) include BB-HB-331.

Endonuclease Modulators

Examples of endonuclease modulators include PGN-514.

Ribonucleotide Reductase Inhibitors

Examples of inhibitors of ribonucleotide reductase include Trimidox.

HBV E Antigen Inhibitors

Examples of HBV E antigen inhibitors include wogonin.

Covalently Closed Circular DNA (cccDNA) Inhibitors

Examples of cccDNA inhibitors include BSBI-25, and CHR-101.

Farnesoid X Receptor Agonist

Examples of farnesoid x receptor agonist such as EYP-001, GS-9674, EDP-305, MET-409, Tropifexor, AKN-083, RDX-023, BWD-100, LMB-763, INV-3, NTX-023-1, EP-024297 and GS-8670

HBV Antibodies

Examples of HBV antibodies targeting the surface antigens of the hepatitis B virus include GC-1102, XTL-17, XTL-19, KN-003, IV Hepabulin SN, and fully human monoclonal antibody therapy (hepatitis B virus infection, Humabs BioMed).

Examples of HBV antibodies, including monoclonal antibodies and polyclonal antibodies, include Zutectra, Shang Sheng Gan Di, Uman Big (Hepatitis B Hyperimmune), Omri-Hep-B, Nabi-HB, Hepatect CP, HepaGam B, igantibe, Niuliva, CT-P24, hepatitis B immunoglobulin (intravenous, pH4, HBV infection, Shanghai RAAS Blood Products), and Fovepta (BT-088).

Fully human monoclonal antibodies include HBC-34.

CCR2 Chemokine Antagonists

Examples of CCR2 chemokine antagonists include propagermanium.

Thymosin Agonists

Examples of thymosin agonists include Thymalfasin, recombinant thymosin alpha 1 (GeneScience)

Cytokines

Examples of cytokines include recombinant IL-7, CYT-107, interleukin-2 (IL-2, Immunex), recombinant human interleukin-2 (Shenzhen Neptunus), IL-15, IL-21, IL-24, and celmoleukin.

Nucleoprotein Modulators

Nucleoprotein modulators may be either HBV core or capsid protein inhibitors. Examples of nucleoprotein modulators include GS-4882, AB-423, AT-130, GLS4, NVR-1221, NVR-3778, AL-3778, BAY 41-4109, morphothiadine mesilate, ARB-168786, ARB-880, JNJ-379, RG-7907, HEC-72702, AB-506, ABI-H0731, JNJ-440, ABI-H2158 and DVR-23.

Examples of capsid inhibitors include the compounds described in US20140275167 (Novira Therapeutics), US20130251673 (Novira Therapeutics), US20140343032 (Roche), WO2014037480 (Roche), US20130267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), WO2017198744 (Roche), US 20170334882 (Novira), US 20170334898 (Roche), WO2017202798 (Roche), WO2017214395 (Enanta), WO2018001944 (Roche), WO2018001952 (Roche), WO2018005881 (Novira), WO2018005883 (Novira), WO2018011100 (Roche), WO2018011160 (Roche), WO2018011162 (Roche), WO2018011163 (Roche), WO2018036941 (Roche), WO2018043747 (Kyoto Univ), US20180065929 (Janssen), WO2016168619 (Indiana University), WO2016195982 (The Penn State Foundation), WO2017001655 (Janssen), WO2017048950 (Assembly Biosciences), WO2017048954 (Assembly Biosciences), WO2017048962 (Assembly Biosciences), US20170121328 (Novira), US20170121329 (Novira).

Examples of transcript inhibitors include the compounds described in WO2017013046 (Roche), WO2017016960 (Roche), WO2017017042 (Roche), WO2017017043 (Roche), WO2017061466 (Toyoma chemicals), WO2016177655 (Roche), WO2016161268 (Enanta). WO2017001853 (Redex Pharma), WO2017211791 (Roche), WO2017216685 (Novartis), WO2017216686 (Novartis), WO2018019297 (Ginkgo Pharma), WO2018022282 (Newave Pharma), US20180030053 (Novartis), WO2018045911 (Zhejiang Pharma).

Retinoic Acid-inducible Gene 1 Stimulators

Examples of stimulators of retinoic acid-inducible gene 1 include SB-9200, SB-40, SB-44, ORI-7246, ORI-9350, ORI-7537, ORI-9020, ORI-9198, and ORI-7170, RGT-100.

NOD2 Stimulators

Examples of stimulators of NOD2 include SB-9200.

Phosphatidylinositol 3-Kinase (PI3K) Inhibitors

Examples of PI3K inhibitors include idelalisib, ACP-319, AZD-8186, AZD-8835, buparlisib, CDZ-173, CLR-457, pictilisib, neratinib, rigosertib, rigosertib sodium, EN-3342, TGR-1202, alpelisib, duvelisib, IPI-549, UCB-5857, taselisib, XL-765, gedatolisib, ME-401, VS-5584, copanlisib, CAI orotate, perifosine, RG-7666, GSK-2636771, DS-7423, panulisib, GSK-2269557, GSK-2126458, CUDC-907, PQR-309, INCB-40093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301, TAK-117, HMPL-689, tenalisib, voxtalisib, and CLR-1401.

Indoleamine-2, 3-dioxygenase (IDO) Pathway Inhibitors

Examples of IDO inhibitors include epacadostat (INCB24360), resminostat (4SC-201), indoximod, F-001287, SN-35837, NLG-919, GDC-0919, GBV-1028, GBV-1012, NKTR-218, and the compounds described in US20100015178 (Incyte), US2016137652 (Flexus Biosciences, Inc.), WO2014073738 (Flexus Biosciences, Inc.), and WO2015188085 (Flexus Biosciences, Inc.).

PD-1 Inhibitors

Examples of PD-1 inhibitors include cemiplimab, nivolumab, pembrolizumab, pidilizumab, BGB-108, STI-A1014, SHR-1210, PDR-001, PF-06801591, IBI-308, GB-226, STI-1110, JNJ-63723283, CA-170, durvalumab, atezolizumab and mDX-400, JS-001, Camrelizumab, Sintilimab, Sintilimab, tislelizumab, BCD-100, BGB-A333 JNJ-63723283, GLS-010 (WBP-3055), CX-072, AGEN-2034, GNS-1480 (Epidermal growth factor receptor antagonist; Programmed cell death ligand 1 inhibitor), CS-1001, M-7824 (PD-L1/TGF-0 bifunctional fusion protein), Genolimzumab, BMS-936559.

PD-L1 Inhibitors

Examples of PD-L1 inhibitors include atezolizumab, avelumab, AMP-224, MEDI-0680, RG-7446, GX-P2, durvalumab, KY-1003, KD-033, MSB-0010718C, TSR-042, ALN-PDL, STI-A1014, GS-4224, CX-072, and BMS-936559.

Examples of PD-1 inhibitors include the compounds described in WO2017112730 (Incyte Corp), WO2017087777 (Incyte Corp), WO2017017624, WO2014151634 (Bristol Myers Squibb Co), WO201317322 (Bristol Myers Squibb Co), WO2018119286 (Incyte Corp), WO2018119266 (Incyte Corp), WO2018119263 (Incyte Corp), WO2018119236 (Incyte Corp), WO2018119221 (Incyte Corp), WO2018118848 (Bristol Myers Squibb Co), WO20161266460 (Bristol Myers Squibb Co), WO2017087678 (Bristol Myers Squibb Co), WO2016149351 (Bristol Myers Squibb Co), WO2015033299 (Aurigene Discovery Technologies Ltd), WO2015179615 (Eisai Co Ltd; Eisai Research Institute), WO2017066227 (Bristol Myers Squibb Co), WO2016142886 (Aurigene Discovery Technologies Ltd), WO2016142852 (Aurigene Discovery Technologies Ltd), WO2016142835 (Aurigene Discovery Technologies Ltd; Individual), WO2016142833 (Aurigene Discovery Technologies Ltd), WO2018085750 (Bristol Myers Squibb Co), WO2015033303 (Aurigene Discovery Technologies Ltd), WO2017205464 (Incyte Corp), WO2016019232 (3M Co; Individual; Texas A&M University System), WO2015160641 (Bristol Myers Squibb Co), WO2017079669 (Incyte Corp), WO2015033301 (Aurigene Discovery Technologies Ltd), WO2015034820 (Bristol Myers Squibb Co), WO2018073754 (Aurigene Discovery Technologies Ltd), WO2016077518 (Bristol Myers Squibb Co), WO2016057624 (Bristol Myers Squibb Co), WO2018044783 (Incyte Corp), WO2016100608 (Bristol Myers Squibb Co), WO2016100285 (Bristol Myers Squibb Co), WO2016039749 (Bristol Myers Squibb Co), WO2015019284 (Cambridge Enterprise Ltd), WO2016142894 (Aurigene Discovery Technologies Ltd), WO2015134605 (Bristol Myers Squibb Co), WO2018051255 (Aurigene Discovery Technologies Ltd), WO2018051254 (Aurigene Discovery Technologies Ltd), WO2017222976 (Incyte Corp), WO2017070089 (Incyte Corp), WO2018044963 (Bristol Myers Squibb Co), WO2013144704 (Aurigene Discovery Technologies Ltd), WO2018013789 (Incyte Corp), WO2017176608 (Bristol Myers Squibb Co), WO2018009505 (Bristol Myers Squibb Co), WO2011161699 (Aurigene Discovery Technologies Ltd), WO2015119944 (Incyte Corp; Merck Sharp & Dohme Corp), WO2017192961 (Incyte Corp), WO2017106634 (Incyte Corp), WO2013132317 (Aurigene Discovery Technologies Ltd), WO2012168944 (Aurigene Discovery Technologies Ltd), WO2015036927 (Aurigene Discovery Technologies Ltd), WO2015044900 (Aurigene Discovery Technologies Ltd), and WO2018026971 (Arising International), and GS-4224.

Other examples of PD-1 and/or PDL-1 inhibitors include the compounds described in U.S. Provisional Ser. Nos. 62/630,187, 62/640,534, 62/736,116, and 62/747,029.

Recombinant Thymosin Alpha-1

Examples of recombinant thymosin alpha-1 include NL-004 and PEGylated thymosin alpha-1.

Bruton's Tyrosine Kinase (BTK) Inhibitors

Examples of BTK inhibitors include ABBV-105, acalabrutinib (ACP-196), ARQ-531, BMS-986142, dasatinib, ibrutinib, GDC-0853, PRN-1008, SNS-062, ONO-4059, BGB-3111, ML-319, MSC-2364447, RDX-022, X-022, AC-058, RG-7845, spebrutinib, TAS-5315, TP-0158, TP-4207, HM-71224, KBP-7536, M-2951, TAK-020, AC-0025, and the compounds described in US20140330015 (Ono Pharmaceutical), US20130079327 (Ono Pharmaceutical), and US20130217880 (Ono Pharmaceutical).

KDM Inhibitors

Examples of KDM5 inhibitors include the compounds described in WO2016057924 (Genentech/Constellation Pharmaceuticals), US20140275092 (Genentech/Constellation Pharmaceuticals), US20140371195 (Epitherapeutics) and US20140371214 (Epitherapeutics), US20160102096 (Epitherapeutics), US20140194469 (Quanticel), US20140171432, US20140213591 (Quanticel), US20160039808 (Quanticel), US20140275084 (Quanticel), WO2014164708 (Quanticel).

Examples of KDM1 inhibitors include the compounds described in U.S. Pat. No. 9,186,337B2 (Oryzon Genomics), GSK-2879552, and RG-6016.

STING Agonists

Examples of STING agonists include SB-11285, AdVCA0848, STINGVAX, and the compounds described in WO 2018065360 (“Biolog Life Science Institute Forschungslabor und Biochemica-Vertrieb GmbH, Germany), WO 2018009466 (Aduro Biotech), WO 2017186711 (InvivoGen), WO 2017161349 (Immune Sensor), WO 2017106740 (Aduro Biotech), US 20170158724 (Glaxo Smithkiline), WO 2017075477 (Aduro Biotech), US 20170044206 (Merck), WO 2014179760 (University of California), WO2018098203 (Janssen), WO2018118665 (Merck), WO2018118664 (Merck), WO2018100558 (Takeda), WO2018067423 (Merck), WO2018060323 (Boehringer).

Non-Nucleoside Reverse Transcriptase Inhibitors (NNRTI)

Examples of NNRTI include the compounds described in WO2018118826 (Merck), WO2018080903 (Merck), WO2018119013 (Merck), WO2017100108 (Idenix), WO2017027434 (Merck), WO2017007701 (Merck), WO2008005555 (Gilead).

HBV Replication Inhibitors

Examples of hepatitis B virus replication inhibitors include isothiafludine, IQP-HBV, RM-5038, and Xingantie.

Arginase Inhibitors

Examples of Arginase inhibitors include CB-1158, C-201, and resminostat.

Gene Therapy and Cell Therapy

Gene therapy and cell therapy includes the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; and genetic approaches to modify cellular activity to further alter endogenous immune responsiveness against the infection.

Gene Editors

Examples of genome editing systems include a CRISPR/Cas9 system, a zinc finger nuclease system, a TALEN system, a homing endonucleases system, and a meganuclease system; e.g., cccDNA elimination via targeted cleavage, and altering one or more of the hepatitis B virus (HBV) viral genes. Altering (e.g., knocking out and/or knocking down) the PreC, C, X PreSI, PreS2, S, P or SP gene refers to (1) reducing or eliminating PreC, C, X PreSI, PreS2, S, P or SP gene expression, (2) interfering with Precore, Core, X protein, Long surface protein, middle surface protein, S protein (also known as HBs antigen and HBsAg), polymerase protein, and/or Hepatitis B spliced protein function (HBe, HBc, HBx, PreS1, PreS2, S, Pol, and/or HBSP or (3) reducing or eliminating the intracellular, serum and/or intraparenchymal levels of HBe, HBc, HBx, LHBs, MHBs, SHBs, Pol, and/or HBSP proteins. Knockdown of one or more of the PreC, C, X PreSI, PreS2, S, P and/or SP gene(s) is performed by targeting the gene(s) within HBV cccDNA and/or integrated HBV DNA.

CAR-T Cell Therapy

CART cell therapy includes a population of immune effector cells engineered to express a chimeric antigen receptor (CAR), wherein the CAR comprises an HBV antigen-binding domain. The immune effector cell is a T cell or an NK cell. In some embodiments, the T cell is a CD4+ T cell, a CD8+ T cell, or a combination thereof. Cells can be autologous or allogeneic.

TCR-T Cell Therapy

TCR T cell therapy includes T cells expressing HBV-specific T cell receptors. TCR-T cells are engineered to target HBV derived peptides presented on the surface of virus-infected cells. In some embodiments, the T-cells express HBV surface antigen (HBsAg)-specific TCR. Examples of TCR-T therapy directed to treatment of HBV include LTCR-H2-1.

In another specific embodiment, a compound described herein, or a pharmaceutically acceptable salt thereof, is combined with an HBV DNA polymerase inhibitor, one or two additional therapeutic agents selected from the group consisting of immunomodulators, TLR modulators, HBsAg inhibitors, HBsAg secretion or assembly inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2, and one or two additional therapeutic agents selected from the group consisting of HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein modulators).

In another specific embodiment, a compound described herein, or a pharmaceutically acceptable salt thereof, is combined with an HBV DNA polymerase inhibitor and at least a second additional therapeutic agent selected from the group consisting of: immunomodulators, TLR modulators, HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, DUOBODIES®, BITES®, XmAbs®, TandAbs®, Fab derivatives, or TCR-like antibodies), cyclophilin inhibitors, stimulators of retinoic acid-inducible gene 1, stimulators of RIG-I like receptors, PD-1 inhibitors, PD-L1 inhibitors, Arginase inhibitors, PI3K inhibitors, IDO inhibitors, and stimulators of NOD2.

In another specific embodiment, a compound described herein, or a pharmaceutically acceptable salt thereof, is combined with an HBV DNA polymerase inhibitor and at least a second additional therapeutic agent selected from the group consisting of: HBV viral entry inhibitors, NTCP inhibitors, HBx inhibitors, cccDNA inhibitors, HBV antibodies targeting the surface antigens of the hepatitis B virus, siRNA, miRNA gene therapy agents, sshRNAs, KDM5 inhibitors, and nucleoprotein modulators (HBV core or capsid protein inhibitors).

In a particular embodiment, a compound described herein, or a pharmaceutically acceptable salt thereof, is combined with compounds such as those described in U.S. Publication No. 2010/0143301 (Gilead Sciences), U.S. Publication No. 2011/0098248 (Gilead Sciences), U.S. Publication No. 2009/0047249 (Gilead Sciences), U.S. Pat. No. 8,722,054 (Gilead Sciences), U.S. Publication No. 2014/0045849 (Janssen), U.S. Publication No. 2014/0073642 (Janssen), WO2014/056953 (Janssen), WO2014/076221 (Janssen), WO2014/128189 (Janssen), U.S. Publication No. 2014/0350031 (Janssen), WO2014/023813 (Janssen), U.S. Publication No. 2008/0234251 (Array Biopharma), U.S. Publication No. 2008/0306050 (Array Biopharma), U.S. Publication No. 2010/0029585 (Ventirx Pharma), U.S. Publication No. 2011/0092485 (Ventirx Pharma), US2011/0118235 (Ventirx Pharma), U.S. Publication No. 2012/0082658 (Ventirx Pharma), U.S. Publication No. 2012/0219615 (Ventirx Pharma), U.S. Publication No. 2014/0066432 (Ventirx Pharma), U.S. Publication No. 2014/0088085 (Ventirx Pharma), U.S. Publication No. 2014/0275167 (Novira Therapeutics), U.S. Publication No. 2013/0251673 (Novira Therapeutics), U.S. Pat. No. 8,513,184 (Gilead Sciences), U.S. Publication No. 2014/0030221 (Gilead Sciences), U.S. Publication No. 2013/0344030 (Gilead Sciences), U.S. Publication No. 2013/0344029 (Gilead Sciences), US20140275167 (Novira Therapeutics), US20130251673 (Novira Therapeutics), U.S. Publication No. 2014/0343032 (Roche), WO2014037480 (Roche), U.S. Publication No. 2013/0267517 (Roche), WO2014131847 (Janssen), WO2014033176 (Janssen), WO2014033170 (Janssen), WO2014033167 (Janssen), WO2015/059212 (Janssen), WO2015118057 (Janssen), WO2015011281 (Janssen), WO2014184365 (Janssen), WO2014184350 (Janssen), WO2014161888 (Janssen), WO2013096744 (Novira), US20150225355 (Novira), US20140178337 (Novira), US20150315159 (Novira), US20150197533 (Novira), US20150274652 (Novira), US20150259324, (Novira), US20150132258 (Novira), U.S. Pat. No. 9,181,288 (Novira), WO2014184350 (Janssen), WO2013144129 (Roche), US20100015178 (Incyte), US2016137652 (Flexus Biosciences, Inc.), WO2014073738 (Flexus Biosciences, Inc.), WO2015188085 (Flexus Biosciences, Inc.), U.S. Publication No. 2014/0330015 (Ono Pharmaceutical), U.S. Publication No. 2013/0079327 (Ono Pharmaceutical), U.S. Publication No. 2013/0217880 (Ono pharmaceutical), WO2016057924 (Genentech/Constellation Pharmaceuticals), US20140275092 (Genentech/Constellation Pharmaceuticals), US20140371195 (Epitherapeutics) and US20140371214 (Epitherapeutics), US20160102096 (Epitherapeutics), US20140194469 (Quanticel), US20140171432, US20140213591 (Quanticel), US20160039808 (Quanticel), US20140275084 (Quanticel), WO2014164708 (Quanticel), U.S. Pat. No. 9,186,337B2 (Oryzon Genomics), and other drugs for treating HBV, and combinations thereof.

### HIV Combination Therapy

In certain embodiments, a method for treating or preventing an HIV infection in a human or animal having or at risk of having the infection is provided, comprising administering to the human or animal a therapeutically effective amount of a composition described herein, in combination with a therapeutically effective amount of one or more (e.g., one, two, three, one or two, or one to three) additional therapeutic agents. In one embodiment, a method for treating an HIV infection in a human or animal having or at risk of having the infection is provided, comprising administering to the human or animal a therapeutically effective amount of a composition described herein, in combination with a therapeutically effective amount of one or more (e.g., one, two, three, one or two, or one to three) additional therapeutic agents.

In certain embodiments, the present description provides a method for treating an HIV infection, comprising administering to a patient in need thereof a therapeutically effective amount of a composition described herein, in combination with a therapeutically effective amount of one or more additional therapeutic agents which are suitable for treating an HIV infection.

In some embodiments, the additional therapeutic agent may be an anti-HIV agent. In some embodiments, the additional therapeutic agent is selected from the group consisting of HIV combination drugs, HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T), latency reversing agents, compounds that target the HIV capsid (including capsid inhibitors), immune-based therapies, phosphatidylinositol 3-kinase (PI3K) inhibitors, alpha-4/beta-7 antagonists, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and other HIV therapeutic agents, or any combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, or any combinations thereof.

HIV Combination Drugs

Examples of combination drugs include ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine+levonorgestrel, dolutegravir+lamivudine, dolutegravir+emtricitabine+tenofovir alafenamide, elsulfavirine+emtricitabine+tenofovir disoproxil, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine; Vacc-4x and romidepsin; and APH-0812, or any combinations thereof.

HIV Protease Inhibitors

Examples of HIV protease inhibitors include amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, and TMC-310911.

HIV Reverse Transcriptase Inhibitors

Examples of HIV non-nucleoside or non-nucleotide inhibitors of reverse transcriptase include dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, and elsulfavirine (VM-1500).

Examples of HIV nucleoside or nucleotide inhibitors of reverse transcriptase include adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, islatravir, lamivudine, phosphazid, stavudine, zalcitabine, zidovudine, rovafovir etalafenamide (GS-9131), GS-9148, MK-8504, MK-8591, MK-858, VM-2500 and KP-1461.

HIV Integrase Inhibitors

Examples of HIV integrase inhibitors include elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169 and cabotegravir.

Examples of HIV non-catalytic site, or allosteric, integrase inhibitors (NCINI) include CX-05045, CX-05168, and CX-14442.

HIV Entry Inhibitors

Examples of HIV entry (fusion) inhibitors include cenicriviroc, CCR5 inhibitors, gp41 inhibitors, CD4 attachment inhibitors, DS-003 (BMS-599793), gp120 inhibitors, and CXCR4 inhibitors.

Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu).

Examples of gp41 inhibitors include albuvirtide, enfuvirtide, BMS-986197, enfuvirtide biobetter, enfuvirtide biosimilar, HIV-1 fusion inhibitors (P26-Bapc), ITV-1, ITV-2, ITV-3, ITV-4, PIE-12 trimer and sifuvirtide.

Examples of CD4 attachment inhibitors include ibalizumab and CADA analogs.

Examples of gp120 inhibitors include Radha-108 (receptol) 3B3-PE38, BanLec, bentonite-based nanomedicine, fostemsavir tromethamine, IQP-0831, and BMS-663068.

Examples of CXCR4 inhibitors include plerixafor, ALT-1188, N15 peptide, and vMIP (Haimipu).

HIV Maturation Inhibitors

Examples of HIV maturation inhibitors include BMS-955176, BMS-986197, GSK-3640254 and GSK-2838232.

Latency Reversing Agents

Examples of latency reversing agents include histone deacetylase (HDAC) inhibitors, proteasome inhibitors such as velcade, and ixazomib citrate, protein kinase C (PKC) activators, Smyd2 inhibitors, BET-bromodomain 4 (BRD4) inhibitors, ionomycin, PMA, SAHA (suberanilohydroxamic acid, or suberoyl, anilide, and hydroxamic acid), AM-0015, ALT-803, NIZ-985, NKTR-255, IL-15 modulating antibodies, JQl, disulfiram, amphotericin B, and ubiquitin inhibitors such as largazole analogs, APH-0812, and GSK-343.

Examples of HDAC inhibitors include romidepsin, vorinostat, and panobinostat.

Examples of PKC activators include indolactam, prostratin, ingenol B, and DAG-lactones.

Capsid Inhibitors

Examples of capsid inhibitors include capsid polymerization inhibitors or capsid disrupting compounds, HIV nucleocapsid p7 (NCp7) inhibitors such as azodicarbonamide, HIV p24 capsid protein inhibitors, GS-6207, AVI-621, AVI-101, AVI-201, AVI-301, and AVI-CAN1-15 series.

HIV Long Acting Agents

Examples of drugs that are being developed as long acting regimens: cabotegravir LA, rilpivirine LA, cabotegravir LA+rilpivirine LA, any integrase LA, VM-1500A-LAI, maraviroc (LAI), tenofovir implant, MK-8591 implant, long-acting dolutegravir, long acting raltegravir+lamivudine.

Immune-Based Therapies

Examples of immune-based therapies include toll-like receptors modulators such as TLR1, TLR2, TLR3, TLR4, TLR5, TLR6, TLR7, TLR8, TLR9, TLR10, TLR11, TLR12, and TLR13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107; interleukin-15/Fc fusion protein; AM-0015, ALT-803, NIZ-985, NKTR-255, NKTR-262, NKTR-214, normferon; peginterferon alfa-2a; peginterferon alfa-2b; recombinant interleukin-15; Xmab-24306, RPI-MN; GS-9620; STING modulators; RIG-I modulators; NOD2 modulators; STING modulators, RIG-I modulators, NOD2 modulators, SB-9200, and IR-103.

Examples of TLR agonists include vesatolimod (GS-9620), GS-986, IR-103, lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod, RO-7020531.

Examples of TLR8 modulators include motolimod, resiquimod, 3M-051, 3M-052, MCT-465, IMO-4200, VTX-763, VTX-1463 and those described in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (VentirxPharma), US20140275167 (Novira therapeutics), US20130251673 (Novira therapeutics), U.S. Pat. No. 9,670,205 (Gilead Sciences Inc.), US20160289229 (Gilead Sciences Inc.), U.S. patent application Ser. No. 15/692,161 (Gilead Sciences Inc.), and U.S. patent application Ser. No. 15/692,093 (Gilead Sciences Inc.)

Phosphatidylinositol 3-Kinase (PI3K) Inhibitors

Examples of PI3K inhibitors include idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

Alpha-4/Beta-7 Antagonists

Examples of Integrin alpha-4/beta-7 antagonists include PTG-100, TRK-170, abrilumab, etrolizumab, carotegrast methyl, and vedolizumab.

HIV Antibodies, Bispecific Antibodies, and “Antibody-Like” Therapeutic Proteins

Examples of HIV antibodies, bispecific antibodies, and “antibody-like” therapeutic proteins include DARTs®, DUOBODIES®, BITES®, XmAbs®, TandAbs®, Fab derivatives, bispecific antibodies, trispecific antibodies, multivalent antibodies, bnABs (broadly neutralizing HIV-1 antibodies), BMS-936559, TMB-360, and those targeting HIV gp120 or gp41, antibody-Recruiting Molecules targeting HIV, anti-CD63 monoclonal antibodies, CD3 bispecific antibodies, CD16 bispecific antibodies, anti-GB virus C antibodies, anti-GP120/CD4, CCR5 bispecific antibodies, anti-nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), ibalizumab, Immuglo, and MB-66.

Further examples include bavituximab, UB-421, C2F5, 2G12, C4E10, C2F5+C2G12+C4E10, 8ANC195, 3BNC117, 3BNC117-LS, D1D2, 3BNC60, 10-1074, 10-1074-LS, GS-9722, DH411-2, BG18, PGT145, PGT121, PGT122, PGT-151, PGT-133, PGT-135, PGT-128, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PG9, PG16, 8ANC195, 2Dm2m, 4Dm2m, 6Dm2m, VRC01, VRC-01-LS, PGDM1400, A32, 7B2, 10E8, 10E8VLS, 3810109, 10E8v4, 10E8.4/iMab, VRC-01/PGDM-1400/10E8v4, IMC-HIV, iMabm36, 10E8v4/PGT121-VRC01, eCD4-Ig, IOMA, CAP256-VRC26.25, DRVIA7, SAR-441236, VRC-07-523, VRC07-523LS, VRC-HIVMAB080-00-AB, VRC-HIVMAB060-00-AB, P2G12, and VRC07. Example of HIV bispecific antibodies include MGD014, TMB-bispecific.

Additional examples of HIV bispecific antibodies include MGD014.

Pharmacokinetic Enhancers

Examples of pharmacokinetic enhancers include cobicistat and ritonavir.

HIV Vaccines

Examples of HIV vaccines include peptide vaccines, recombinant subunit protein vaccines, live vector vaccines using viral vectors such as arenavirus, lymphocytic choriomeningitis virus (LCMV), pichinde virus, modified vaccinia Ankara virus (MVA), adenovirus, adeno-associated virus (AAV), vesicular stomatitis virus (VSV) and Chimpanzee adenovirus (ChAd), DNA vaccines, CD4-derived peptide vaccines, vaccine combinations, BG505 SOSIP.664 gp140, rgp120 (AIDSVAX), ALVAC HIV, (vCP1521)/AIDSVAX B/E (gp120) (RV144), monomeric gp120 HIV-1 subtype C vaccine, Remune, ITV-1, Contre Vir, Ad4-Env145NFL, Ad5-ENVA-48, HB-500, DCVax-001 (CDX-2401), Vacc-4x, Vacc-C5, Vacc-CRX, VVX-004, VAC-3S, multiclade DNA recombinant adenovirus-5 (rAd5), rAd5 gag-pol env A/B/C vaccine, Pennvax-G, Pennvax-GP/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), gp140[delta]V2.TV1+MF-59, rVSVIN HIV-1 gag vaccine, SeV-Gag vaccine, AT-20, DNK-4, ad35-Grin/ENV, TBC-M4, HIVAX, HIVAX-2, NYVAC-HIV-PT1, NYVAC-HIV-PT4, DNA-HIV-PT123, rAAV1-PG9DP, GOVX-B11, GOVX-B21, TVI-HIV-1, Ad-4 (Ad4-env Clade C+Ad4-mGag), Paxvax, EN41-UGR7C, EN41-FPA2, PreVaxTat, AE-H, MYM-V101, CombiHIVvac, ADVAX, MYM-V201, MVA-CMDR, DNA-Ad5 gag/pol/nef/nev (HVTN505), MVATG-17401, ETV-01, CDX-1401, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines, gag-based DNA vaccine, GI-2010, gp41 HIV-1 vaccine, HIV vaccine (PIKA adjuvant), I i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, recombinant peptide vaccine (HIV infection), NCI, rgp160 HIV vaccine, RNActive HIV vaccine, SCB-703, Tat Oyi vaccine, TBC-M4, therapeutic HIV vaccine, UBI HIV gp120, Vacc-4x+romidepsin, variant gp120 polypeptide vaccine, rAd5 gag-pol env A/B/C vaccine, DNA.HTI, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, arenavirus vector-based immunotherapies (Vaxwave, TheraT), MVA-BN HIV-1 vaccine regimen, MVA.tHIVconsv4, MVA.tHIVconsv3, UBI HIV gp120, mRNA based prophylactic vaccines, TBL-1203HI, VRC-HIVRGP096-00-VP, VAX-3S, HIV MAG DNA vaccine.

Additional HIV Therapeutic Agents

Examples of additional HIV therapeutic agents include the compounds described in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

Examples of other drugs for treating HIV include acemannan, alisporivir, astodrimer, BanLec, CC-11050, deferiprone, Gamimune, griffithsin, metenkefalin, naltrexone, Prolastin, REP 9, RPI-MN, Vorapaxar, VSSP, Hlviral, SB-728-T, 1,5-dicaffeoylquinic acid, rHIV7-shl-TAR-CCR5RZ, AAV-eCD4-Ig gene therapy, MazF gene therapy, MK-8527, BlockAide, PSC-RANTES, ABX-464, AG-1105, APH-0812, BIT-225, CYT-107, HGTV-43, HPH-116, HS-10234, IMO-3100, IND-02, MK-1376, MK-2048, MK-4250, MK-8507, MK-8591, NOV-205, PA-1050040 (PA-040), PGN-007, SCY-635, SB-9200, SCB-719, TR-452, TEV-90110, TEV-90112, TEV-90111, TEV-90113, RN-18, Immuglo, and VIR-576.

Gene Therapy and Cell Therapy

Gene therapy and cell therapy include the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; and genetic approaches to modify cellular activity to further alter endogenous immune responsiveness against the infection.

Examples of dendritic cell therapy include AGS-004.

Gene Editors

Examples of gene editing systems include a CRISPR/Cas9 system, a zinc finger nuclease system, a TALEN system, a homing endonucleases system, and a meganuclease system.

Examples of HIV targeting CRISPR/Cas9 systems include EBT101.

CAR-T Cell Therapy

CAR-T cell therapy includes a population of immune effector cells engineered to express a chimeric antigen receptor (CAR), wherein the CAR comprises an HIV antigen-binding domain. The HIV antigens include an HIV envelope protein or a portion thereof, gp120 or a portion thereof, a CD4 binding site on gp120, the CD4-induced binding site on gp120, N glycan on gp120, the V2 of gp120, and the membrane proximal region on gp41. In some embodiments, the immune effector cell is a T cell or an NK cell. In some embodiments, the T cell is a CD4+ T cell, a CD8+ T cell, or a combination thereof.

Examples of HIV CAR-T cell therapy include VC-CAR-T, anti-CD4 CART cell therapy, autologous hematopoietic stem cells genetically engineered to express a CD4 CAR and the C46 peptide.

TCR-T Cell Therapy

TCR-T cell therapy includes T cells engineered to target HIV derived peptides present on the surface of virus-infected cells.

It will be appreciated by one of skill in the art that the additional therapeutic agents listed above may be included in more than one of the classes listed above. The particular classes are not intended to limit the functionality of those compounds listed in those classes.

In a specific embodiment, a compound described herein, or a pharmaceutically acceptable salt thereof, is combined with an HIV nucleoside or nucleotide inhibitor of reverse transcriptase and an HIV non-nucleoside inhibitor of reverse transcriptase. In another specific embodiment, a compound described herein, or a pharmaceutically acceptable salt thereof, is combined with an HIV nucleoside or nucleotide inhibitor of reverse transcriptase, and an HIV protease inhibiting compound. In an additional embodiment, a compound described herein, or a pharmaceutically acceptable salt thereof, is combined with an HIV nucleoside or nucleotide inhibitor of reverse transcriptase, an HIV non-nucleoside inhibitor of reverse transcriptase, and a pharmacokinetic enhancer. In certain embodiments, a compound described herein, or a pharmaceutically acceptable salt thereof, is combined with at least one HIV nucleoside inhibitor of reverse transcriptase, an integrase inhibitor, and a pharmacokinetic enhancer. In another embodiment, a compound described herein, or a pharmaceutically acceptable salt thereof, is combined with two HIV nucleoside or nucleotide inhibitors of reverse transcriptase.

In a particular embodiment, a compound described herein, or a pharmaceutically acceptable salt thereof, is combined with one, two, three, four or more additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); BIKTARVY® (bictegravir, emtricitabine, tenofovir alafenamide); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In a particular embodiment, a compound described herein, or a pharmaceutically acceptable salt thereof, is combined with abacavir sulfate, tenofovir, tenofovir disoproxil, tenofovir disoproxil fumarate, tenofovir disoproxil hemifumarate, tenofovir alafenamide, tenofovir alafenamide hemifumarate, or bictegravir.

In a particular embodiment, a compound described herein, or a pharmaceutically acceptable salt thereof, is combined with tenofovir, tenofovir disoproxil, tenofovir disoproxil fumarate, tenofovir alafenamide, tenofovir alafenamide hemifumarate, or bictegravir.

In a particular embodiment, a compound described herein, or a pharmaceutically acceptable salt thereof, is combined with a first additional therapeutic agent selected from the group consisting of abacavir sulfate, tenofovir, tenofovir disoproxil, tenofovir disoproxil fumarate, tenofovir alafenamide, tenofovir alafenamide hemifumarate, and bictegravir and a second additional therapeutic agent selected from the group consisting of emtricitabine and lamivudine.

In a particular embodiment, a compound described herein, or a pharmaceutically acceptable salt thereof, is combined with a first additional therapeutic agent selected from the group consisting of tenofovir, tenofovir disoproxil, tenofovir disoproxil fumarate, tenofovir alafenamide, tenofovir alafenamide hemifumarate, and bictegravir and a second additional therapeutic agent, wherein the second additional therapeutic agent is emtricitabine.

A compound as described herein may be combined with one or more additional therapeutic agents in any dosage amount of the compound (e.g., from 1 mg to 500 mg of compound).

### x. EXAMPLES

### Example 1. Evaluation of Process Approaches for Encapsulation of the Compound of Formula I in the Biodegradable Polymer Matrix

Three separate process approaches described below were evaluated to encapsulate TAF in the PLGA matrix.

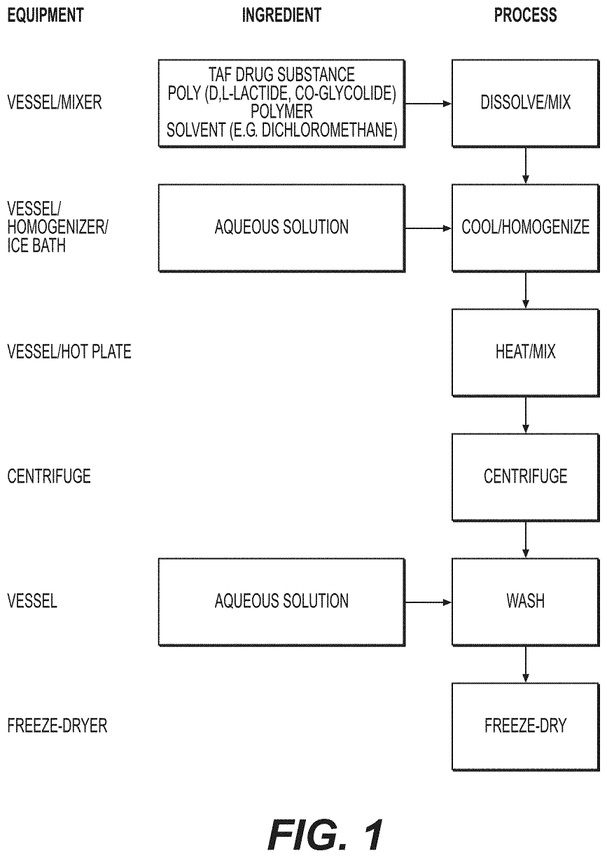

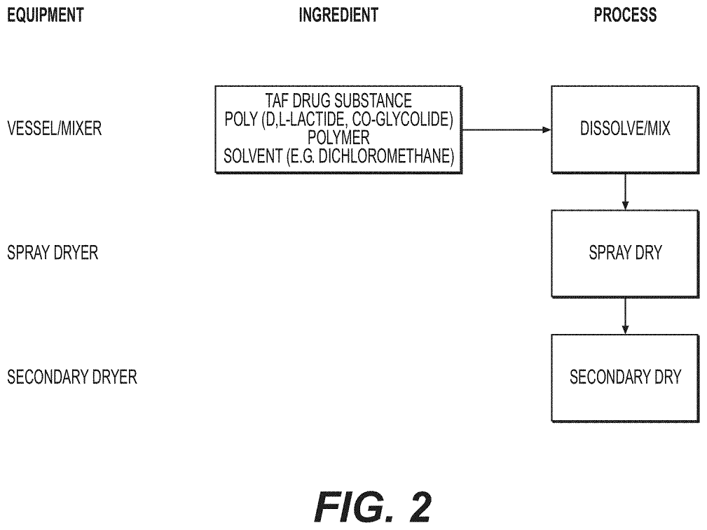

1. Microspheres: To obtain TAF/PLGA microspheres; prototypes with a theoretical TAF loading range of 30% and using PLGA7525 and PDLLA (Pure PLA) were prepared. Crystalline TAF free base (Form I) was used as TAF drug substance. Generally, to prepare an oil phase, TAF drug substance and PLGA were added to a solvent (e.g. dichloromethane and ethanol). The oil phase was added to an aqueous solution in an ice bath and homogenized. The resultant oil-in-water emulsion was transferred to a hot stir plate and mixed. The microspheres were then centrifuged and washed with aqueous solution, and freeze-dried to remove the aqueous solution to yield TAF/PLGA microspheres. From the process, amorphous TAF/PLGA microspheres were obtained. A flow diagram depicting this method of making microspheres is shown in FIG. 1.
2. Spray-dried dispersions: To obtain TAF/PLGA spray dried dispersion (SDD); several prototypes with TAF loading range of 20-50% and using PLGA5050 and PLGA7525 were prepared. Crystalline TAF free base (Form I) and crystalline TAF hemifumarate were used as TAF drug substance. Generally, to obtain a feed solution or a feed suspension for spray drying, TAF drug substance and PLGA were added to a solvent (e.g. dichloromethane, acetone, and ethyl acetate). The feed solution or suspension was spray-dried to yield primary dried TAF/PLGA SDD. The primary dried TAF/PLGA SDD was secondary dried in vacuum oven to remove residual solvents to yield TAF/PLGA SDD. In case of spray drying TAF from feed solution, amorphous TAF/PLGA SDD was obtained. In case of spray drying from suspension formulation, the product was characterized by fused crystals. A flow diagram depicting this method of making microspheres is shown in FIG. 2.
3. Hot-melt extrusion: Hot melt extrusion prototype formulations containing 20-50% TAF loading and 50-80% PLGA (PLGA5050, PLGA7525, PLGA8515, and PDLLA (pure PLA)) were prepared and the manufacturing process is described in Example 2. All the prototype formulations described in Example 3, and onwards, were prepared using the hot melt extrusion process. For Example 2 and onwards, unless specified otherwise, TAF drug substance used was TAF free base (crystalline Form I). Further, for Example 2 and onwards, unless specified otherwise, TAF drug substance was micronized, crystalline Form I of TAF free base.

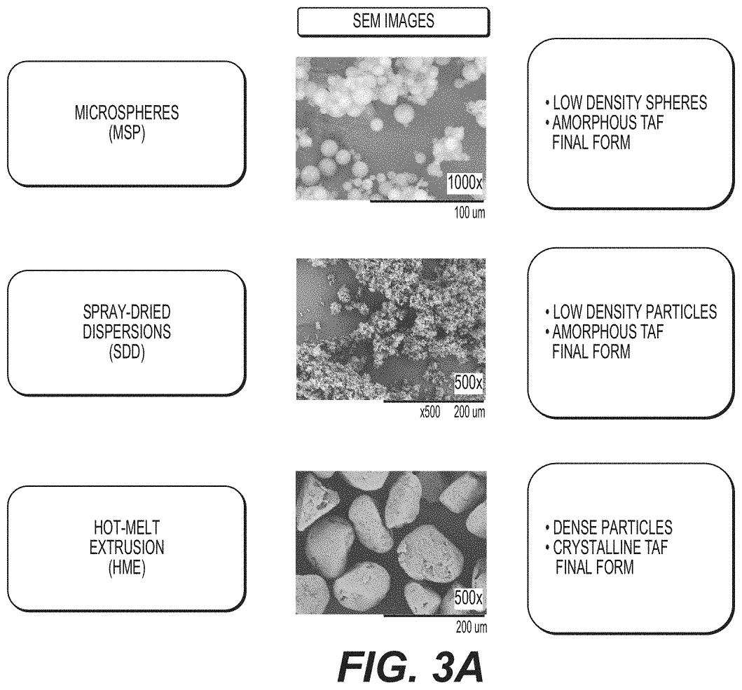

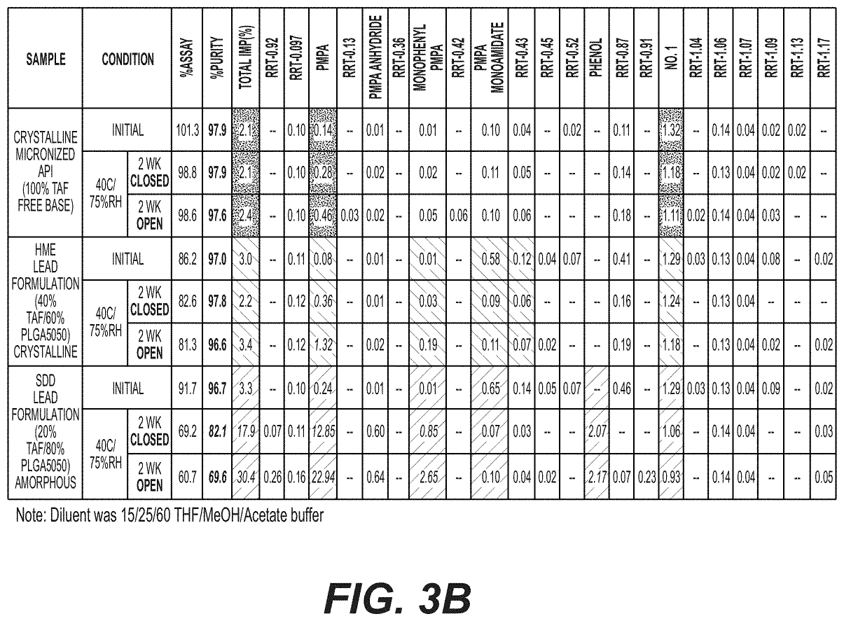

FIG. 3a shows a comparison of the SEM images of the pharmaceutical compositions obtained by the three approaches described above and FIG. 3b shows the results of chemical stability tests of the pharmaceutical compositions obtained by the spray dried dispersion and hot melt extrusion methods described above. The microspheres and the spray-dried dispersions are characterized by amorphous TAF in the final composition, while in the hot melt extruded pharmaceutical composition crystalline form of TAF was retained. Further, the microspheres and the spray-dried dispersions are comprised of low density particles as compared to the hot-melt extruded compositions. As seen in FIG. 3b, TAF in SDD formulations were less chemically stable (17.9% total impurities observed at 40° C./75% RH closed for 2 weeks) as compared to the microspheres and the hot-melt extruded compositions (2.2% total impurities observed at 40° C./75% RH closed for 2 weeks). As a reference, TAF API alone had 2.1% total impurities observed initially and maintained that level of impurities at 40° C./75% RH closed for 2 weeks. [No. 1 in FIG. 3b is isopropyl ((R)—((((R)-1-(6-amino-9H-purin-9-yl)propan-2-yl)oxy)methyl) (phenoxy)phosphoryl)-L-alaninate]

TAF loading in the microsphere formulations were low (3-9%) as opposed to TAF loading in HME formulations (20-50%).

### Example 2. Process for Making Hot Melt Extruded Composition of TAF Drug Substance (TAF Powder for Injection (TAF PI))

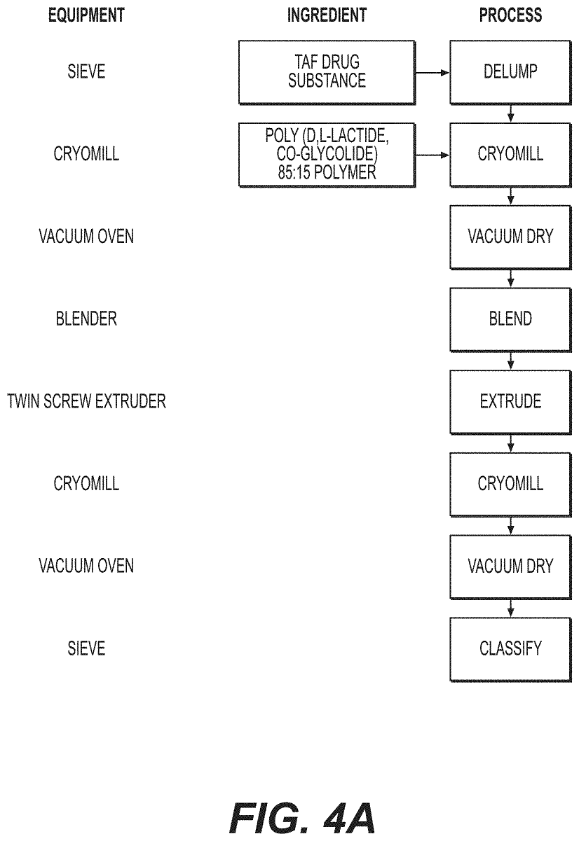

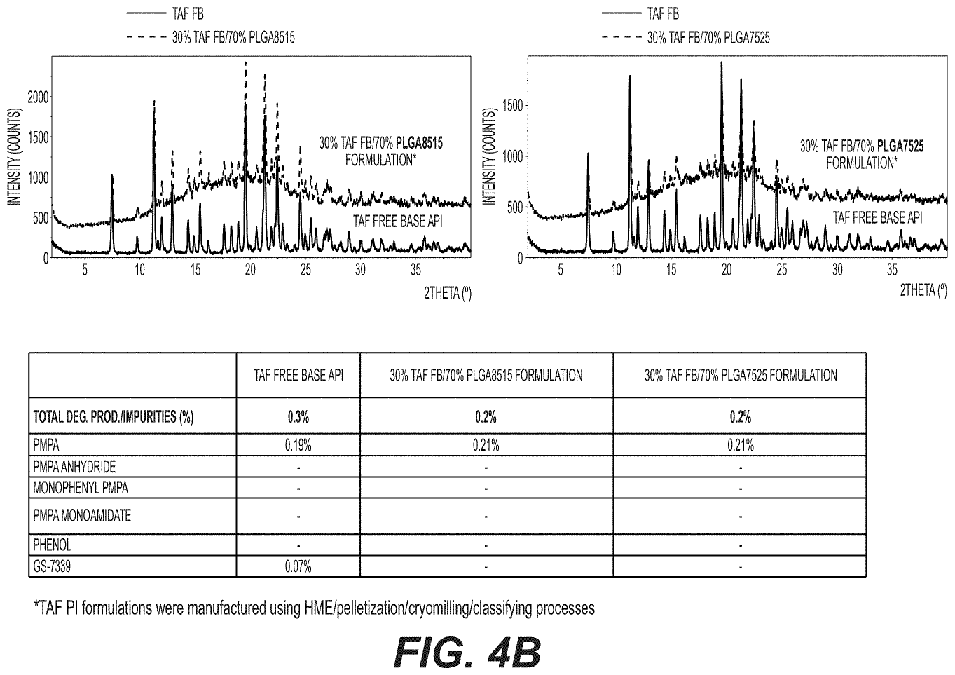

A flow diagram depicting the complete manufacturing process for TAF PI, is shown in FIG. 4a.

In a representative example, Tenofovir alafenamide (in the form of crystalline Form I of the TAF free base) was micronized to obtain d₉₀<10 μm and delumped. The Poly (D, L-lactide, co-glycolide) 85:15 Polymer (PLGA8515) was cryomilled and dried. The delumped tenofovir alafenamide and milled PLGA8515 were blended and extruded to yield TAF hot melt particles (HMP) with composition of 19% TAF and 81% PLGA8515 w/w. TAF HMP was cryomilled, dried, and classified. The resulting bulk powder was filled into 6R Type I clear glass vials and fitted with coated rubber stoppers and aluminum seals with polypropylene flip-off caps. The vials were then terminally sterilized by gamma irradiation.

Qualitative and Quantitative Composition of the exemplary
TAF PI

| | Composition | Unit Formula |
|---|---|---|
| Components | (% w/w) | (mg/unit) |
| Tenofovir Alafenamide^{a} | 19.0 | 150.1 |
| Poly (D, L-lactide, co-glycolide) | 81.0 | 639.9 |
| 85:15 Polymer^{a} | | |
| Total | 100.0 | 790 |

^{a}The actual quantity of tenofovir alafenamide was adjusted based on the drug content factor (DCF) with a concomitant adjustment in the quantity of Poly (D, L-lactide, co-glycolide) 85:15 Polymer. The quantity of tenofovir alafenamide may include an overage to account for processing losses.

Further examples of the pharmaceutical compositions that were prepared by this method include (i) 30% TAF (free base, crystalline Form I, and micronized) and 70% PLGA8515 and (ii) 30% TAF (free base, crystalline Form I, and micronized) and 70% PLGA7525. FIG. 4b shows an overlap of the XRPD spectra of these compositions with the XRPD spectra of the crystalline Form I of the TAF free base starting material. As seen, the crystalline nature of the TAF free base is retained through the hot melt extrusion procedure.

### Example 3. Impact of the Physical Form of the Compound of Formula I on the Sustained Release Formulation

Several TAF crystalline forms (free base and salts) with different aqueous solubility (0.2 to 3.5 mg/mL) were formulated as 30% drug load/70% PLGA7525 or PLGA8515 formulations. The forms/salts formulated and their solubilities are summarized below:

| TAF form | Solubility in water |
|---|---|
| Free base | ~3.5 mg/mL |
| Orotate salt | ~1.7 mg/mL |
| Vanillate salt | ~1.6 mg/mL |
| Sebacate salt | ~0.7 mg/mL |
| Bis-xinafoate salt | ~0.2 mg/mL |

These formulations were evaluated in vitro (PBS, pH 7.4, 37° C.) for chemical stability and in dog model for Pharmacokinetic (PK) performance. All formulations were dosed at 200 mg/mL solid concentration. Suspension vehicle for all the formulations was 0.6% HPMC (K4M), 0.2% Tween 80, 99.2% PBS pH 7.4. All formulations were dosed subcutaneously using 19 G TW or 18 G R needle size.

Formulations containing 30% TAF free base (crystalline Form I, micronized)/70% PLGA7525 and 30% TAF bis-xinafoate (crystalline, micronized)/70% PLGA7525 were evaluated in vitro (PBS, pH 7.4, 37° C.) for chemical stability and in vivo dog PK study. The results of these studies are shown in FIG. 5. As seen in FIG. 5, no significant differences were observed in both in vitro chemical stability and in vivo PK performance of two formulations.

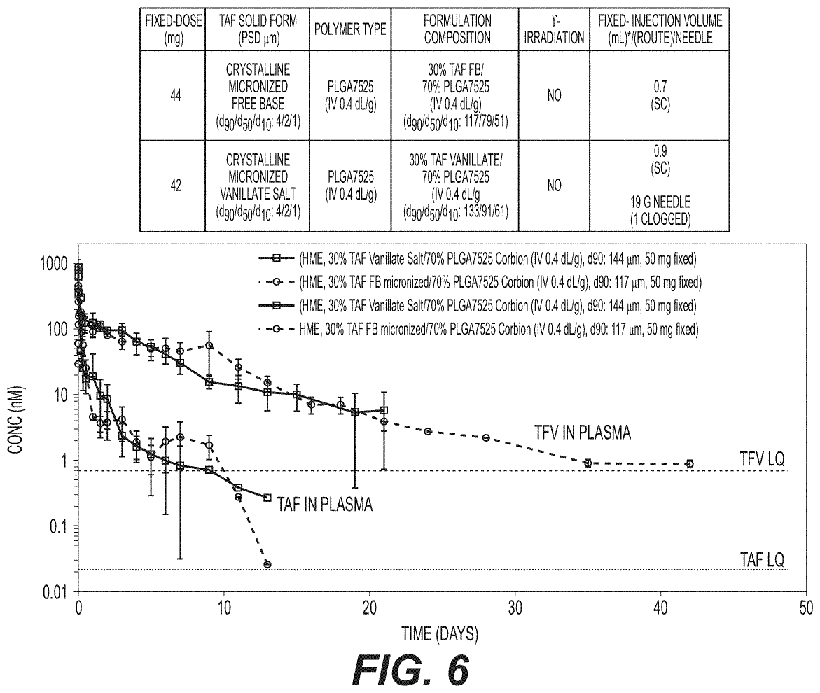

Formulations containing 30% TAF free base (crystalline Form I, micronized)/70% PLGA7525 and 30% TAF vanillate (crystalline, micronized)/70% PLGA7525 were evaluated for PK performance in dog model. The results of these experiments are shown in FIG. 6. As seen in FIG. 6, no significant differences were observed in both in vitro chemical stability and in vivo PK performance of two formulations.

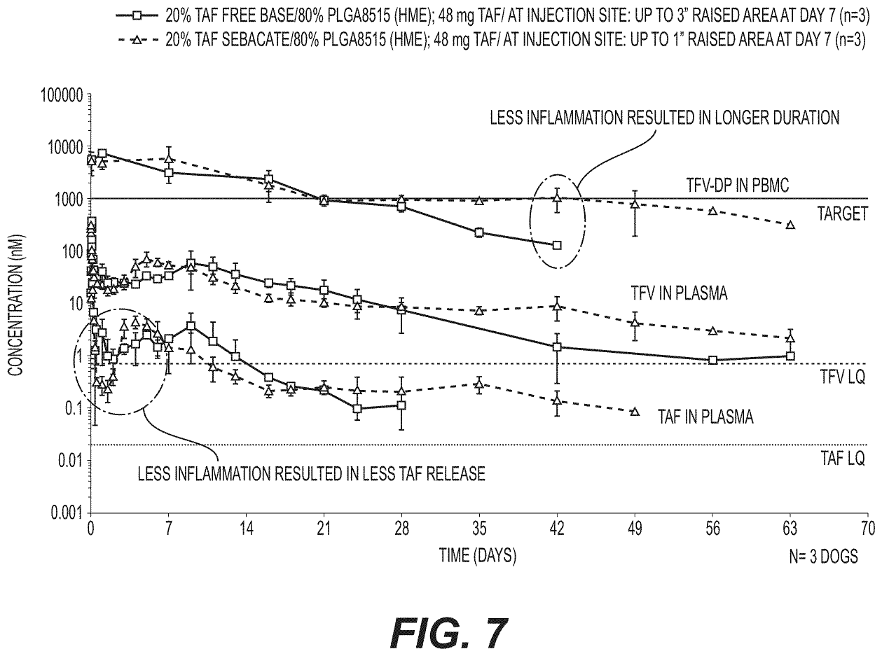

Formulations containing 20% TAF free base (crystalline Form I, micronized)/80% PLGA8515 and 20% TAF sebacate (crystalline Form I, micronized)/80% PLGA8515 were evaluated for PK performance in dog model. The results of these experiments are shown in FIG. 7. As, seen in FIG. 7, the formulation containing the TAF sebacate showed longer duration of TAF and TFV concentration in plasma (49 days of TAF and 63 days of TFV) compared to the formulation containing TAF free base (28 days for TAF and 48 days for TFV). Additionally, the TFV-DP concentration in PBMCs (HIV-target cells) (0.798 μM at 49 days) compared to the formulation containing TAF free base (0.716 μM at 28 days). Also, a lower initial burst and less inflammation was observed for the TAF sebacate containing formulation (1″ raised area at injection site on day 7) compared to the TAF free base formulation (3″ raised area at injection site on day 7), indicating TAF sebacate has anti-inflammatory properties.

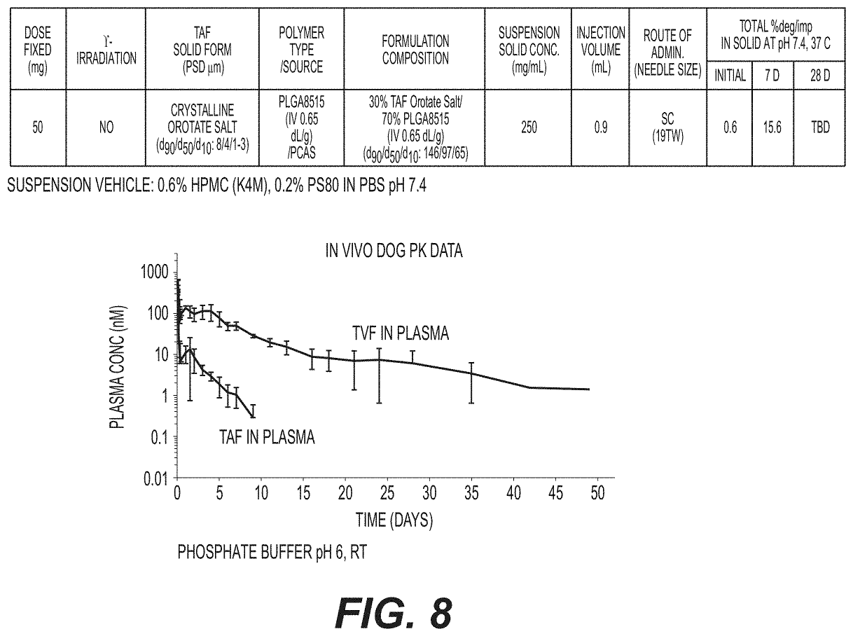

Formulation containing 30% TAF orotate (crystalline Form I, micronized)/70% PLGA8515 was manufactured and evaluated in vitro (PBS, pH 7.4, 37° C.) for chemical stability and in vivo dog PK study. The results of this Experiments are shown in FIG. 8. As seen in FIG. 8, this formulation did not maintain a long duration of TAF concentration in plasma (about 9 days).

### Example 4. Impact of TAF Free Base Physical Form

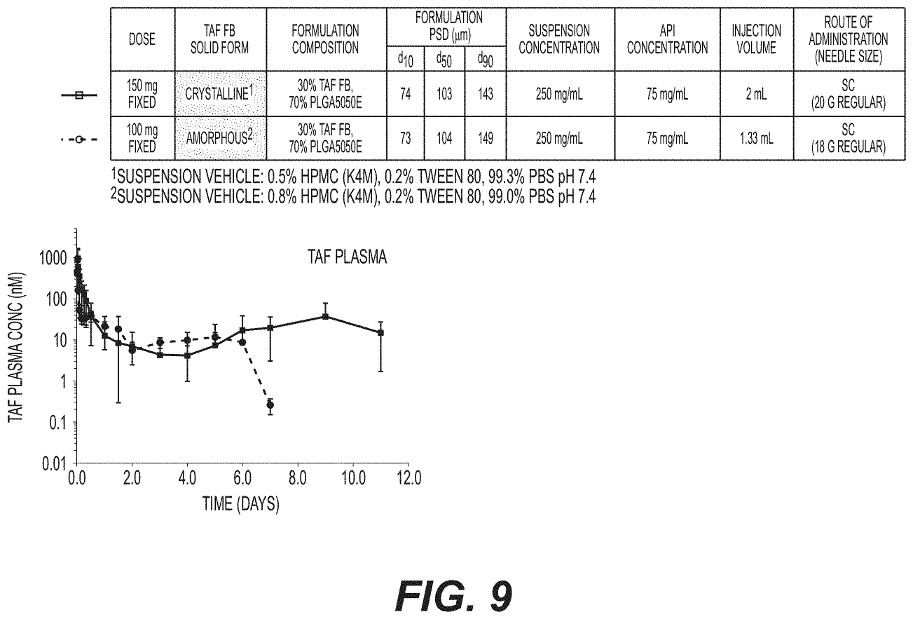

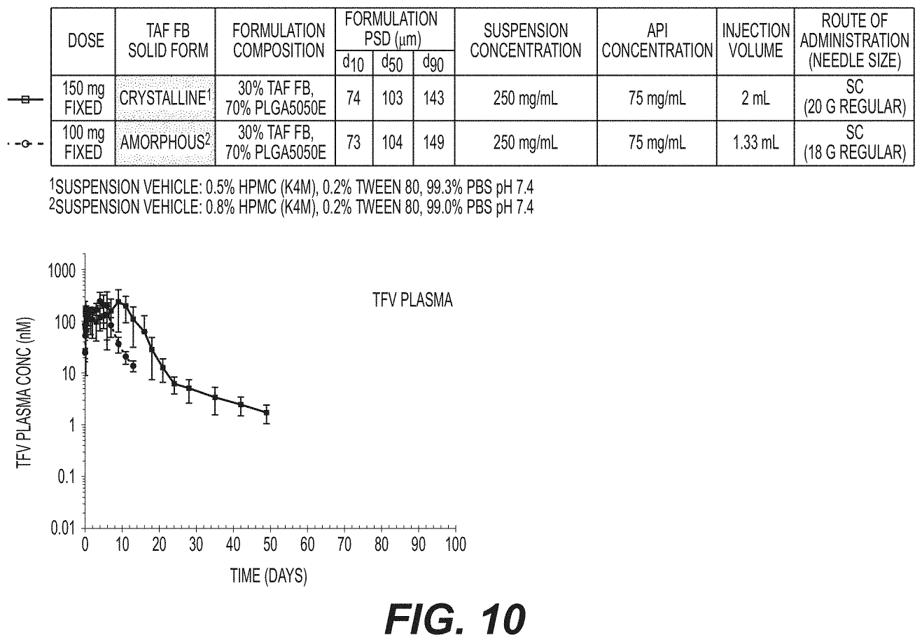

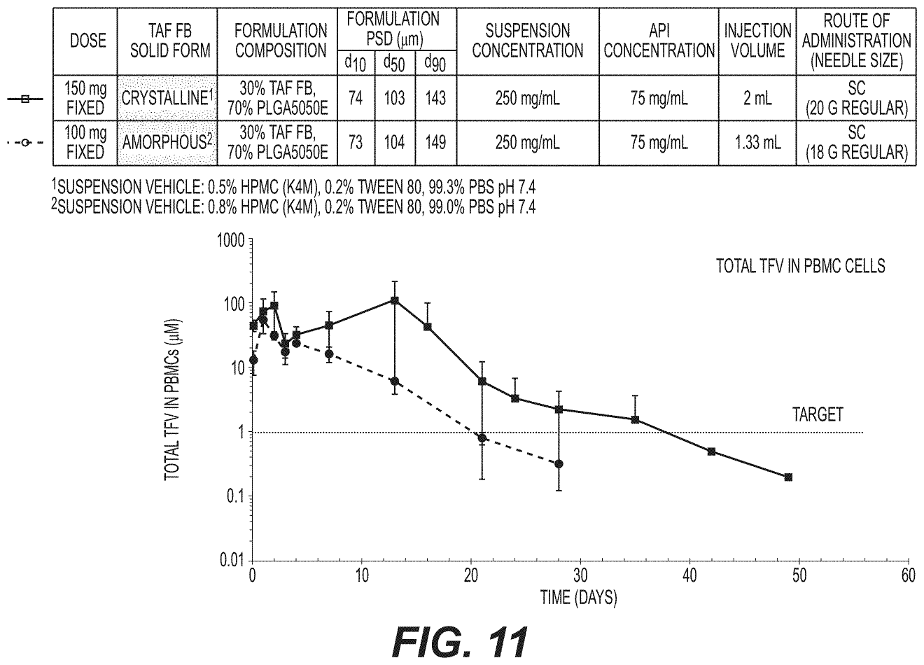

Formulations comprising 30% TAF and 70% PLGA5050 were prepared using TAF free base in crystalline (Form I) and amorphous forms. These formulations were evaluated in vitro and evaluated in dog model for PK performance. The results of these experiments are shown in FIG. 9-FIG. 11. As seen in FIGS. 9 and 10, the formulations comprising crystalline TAF free base maintained a longer duration of TAF and TFV in plasma. Likewise, as seen in FIG. 11, the formulation containing crystalline TAF free base maintained total TFV concentration ≥1 μM in PBMCs (HIV-target cells) for a longer time (35 days) as compared to the formulation containing amorphous TAF free base (20 days).

### Example 5. Impact TAF Particle Size

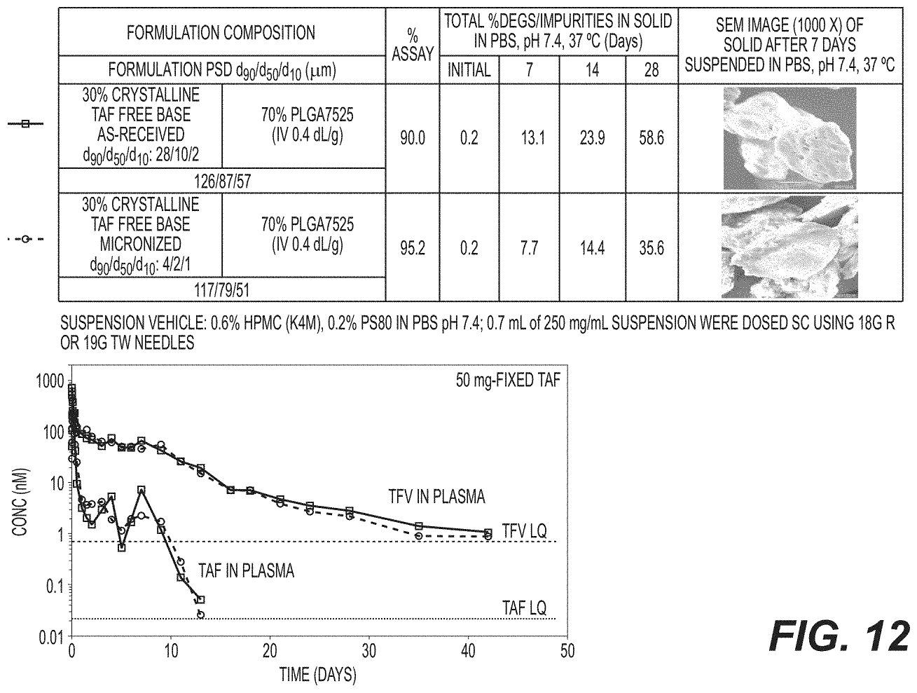

Formulations comprising 30% TAF and 70% PLGA7525 were prepared using (i) micronized (d₉₀<10 μm) and (ii) unmicronized (d₉₀=28 μm) crystalline Form I of TAF free base. The two formulations were evaluated in vitro (PBS, pH 7.4, 37° C.) and in vivo dog PK study. The results of these experiments are shown in FIG. 12. As seen in FIG. 12, the formulation containing micronized crystalline TAF free base was chemically more stable than the formulation containing unmicronized TAF free base. The in vivo dog PK performance of the two formulations was comparable.

### Example 6. Impact of the Pharmaceutical Composition Particle Size

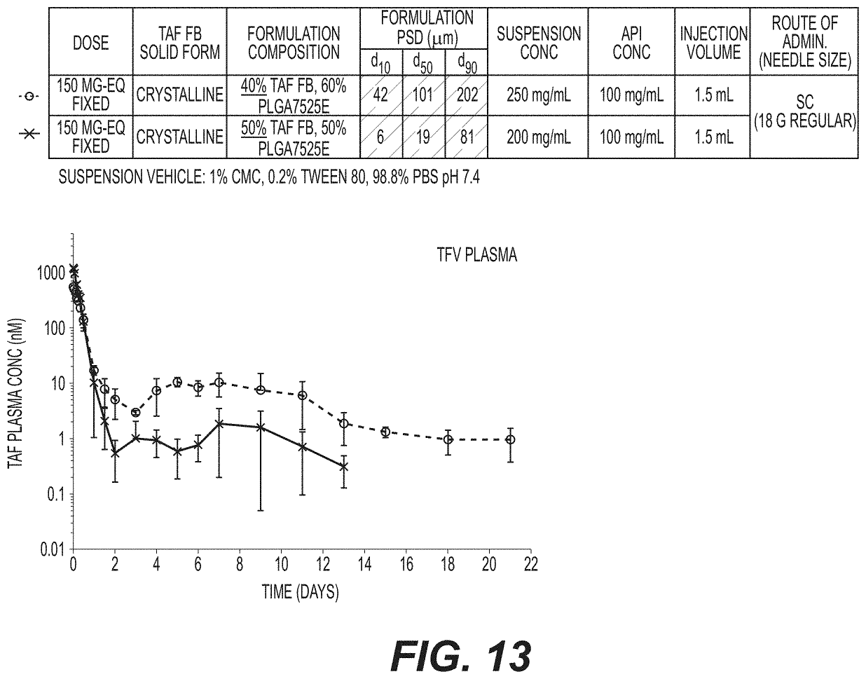

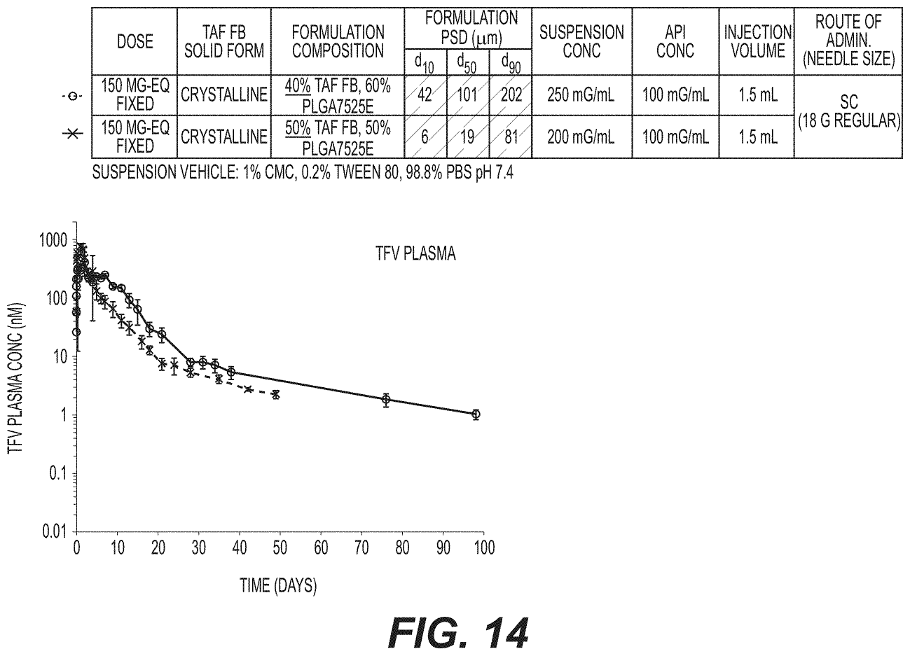

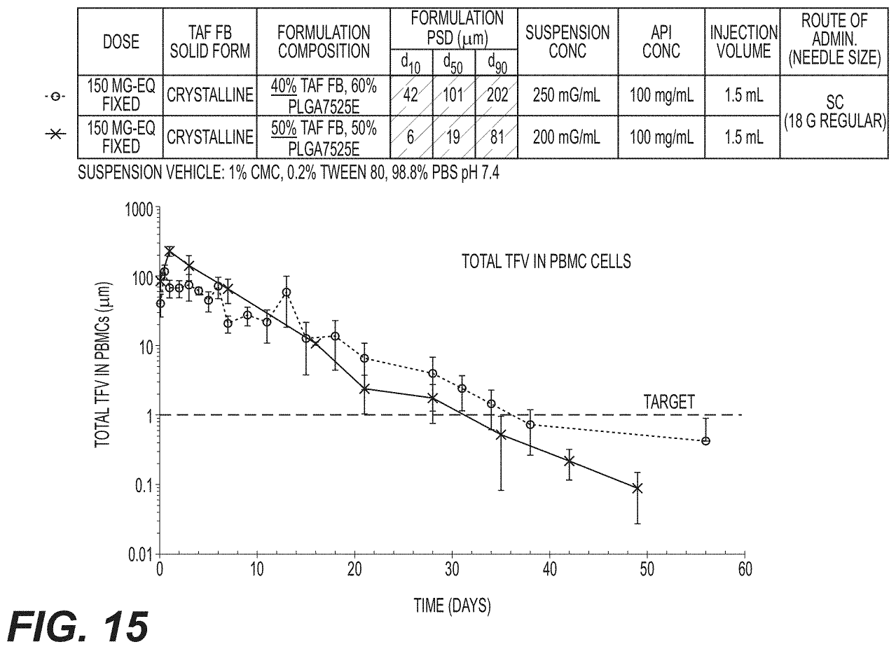

The following two pharmaceutical compositions were prepared and evaluated in dog PK study: (i) 40% TAF (crystalline free base, micronized) and 60% PLGA7525 with the composition particle size of d₁₀=42 μm, d₅₀=101 μm, and d₉₀=202 μm and (ii) 50% TAF (crystalline free base, micronized) and 50% PLGA7525 with the composition particle size of d₁₀=6 μm, d₅₀=19 μm, and d₉₀=81 μm. The results of these experiments are shown in FIGS. 13-15. As seen, the formulation with a larger particle size (d₁₀: 42 μm/d₉₀: 202 μm) showed a longer duration of TAF and TFV in plasma (FIGS. 13 and 14). This composition also demonstrated slower in vivo burst (the initial bolus of the TAF drug substance that is released before the release rate reaches a stable profile) and maintained total TFV concentration ≥1 μM in PBMCs for a longer time (34 days) as compared to the formulation with a smaller particle size (d₁₀: 6 μm/d₉₀: 81 μm) (28 days) (FIG. 15).

Additionally, the impact of the composition particle size on the formulation syringeability was investigated. 40% TAF (crystalline free base, micronized) and 60% PLGA5050 formulation with two different particle sizes (i) d₁₀=9 μm and d₉₀=129 μm and (ii) d₁₀=40 μm and d₉₀: 222 μm were evaluated for syringeability. The smaller particle size formulation (d₁₀=9 μm and d₉₀=129 μm) passed through a smaller needle gauge (20 G) as compared to the larger particle size formulation which passed through the larger needle gauge (18 G).

Particle size d₁₀>50 μm and d₉₀≤155 μm showed an optimal PK performance in a dog model and suitable syringeability.

### Example 7. Evaluation of Polymer Types

Formulations containing various grades of PLGA (PLA:PGA ratio): PLGA5050; PLGA7525, PLGA8515; and PDLLA (pure PLA) with TAF loading range of 20-50% were evaluated in dog PK study. The in vivo degradation times for these polymers ranged from 1 to 12-16 months.

A comparison of three formulations containing 30% (free base, crystalline, and micronized) TAF and 70% PLGA7525, PLGA8515, or PDLLA, showed no significant differences in PK profiles. The PK variability seen from PLGA8515 formulation was lower compared to PLGA7525 formulation. PDLLA has a 12-18 months degradation time in vivo.

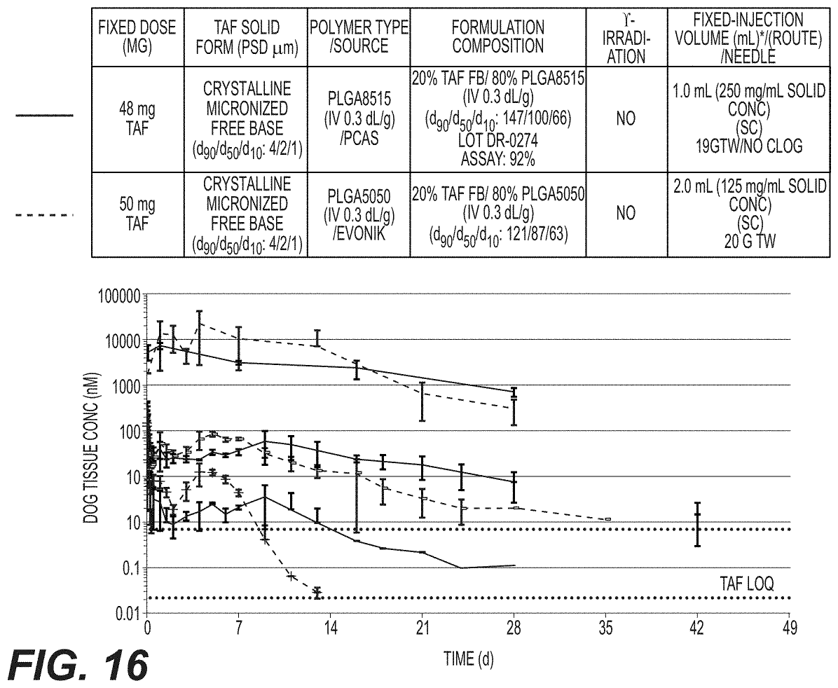

Further, two formulations containing (i) 20% TAF free base (crystalline Form I, micronized) and 80% PLGA8515 and (ii) 20% TAF free base (crystalline Form I, micronized) and 80% PLGA5050 were evaluated for PK performance in dog model. Results from these experiments are summarized in FIG. 16. As seen in FIG. 16, the formulation containing PLGA8515 showed a higher level of TFV-DP concentration in PBMCs (HIV-target cells) at day 28 (0.72 μM) compared to the formulation containing PLGA5050 (0.31 μM). Additionally, the formulation containing PLGA8515 also showed a longer duration of TAF concentration in plasma (28 days) compared to the formulation containing PLGA5050 (13 days).

### Example 8. Comparison of Drug Loading on TAF Extended Release Formulation

Formulations containing various TAF loading range of 20-50% and using PLGA (PLA:PGA ratio): PLGA50:50; PLGA75:25, PLGA85:15; and PDLLA (Pure PLA) were manufactured.

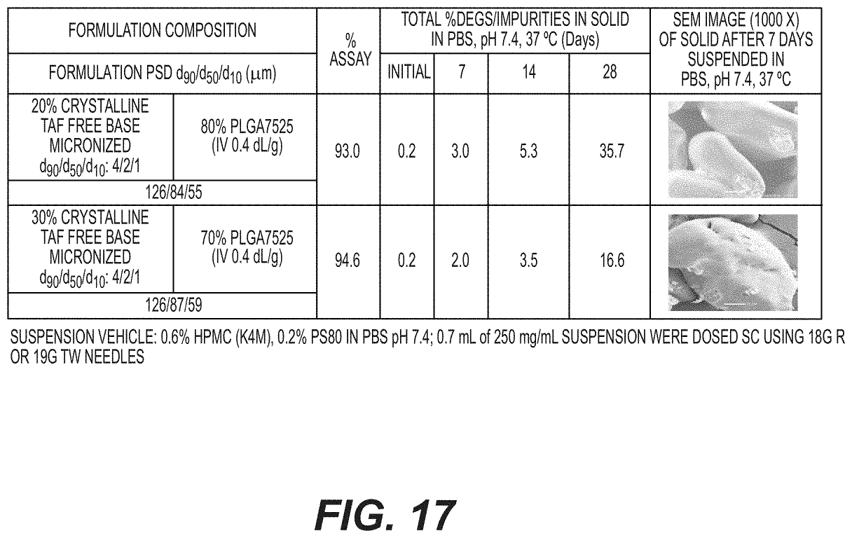

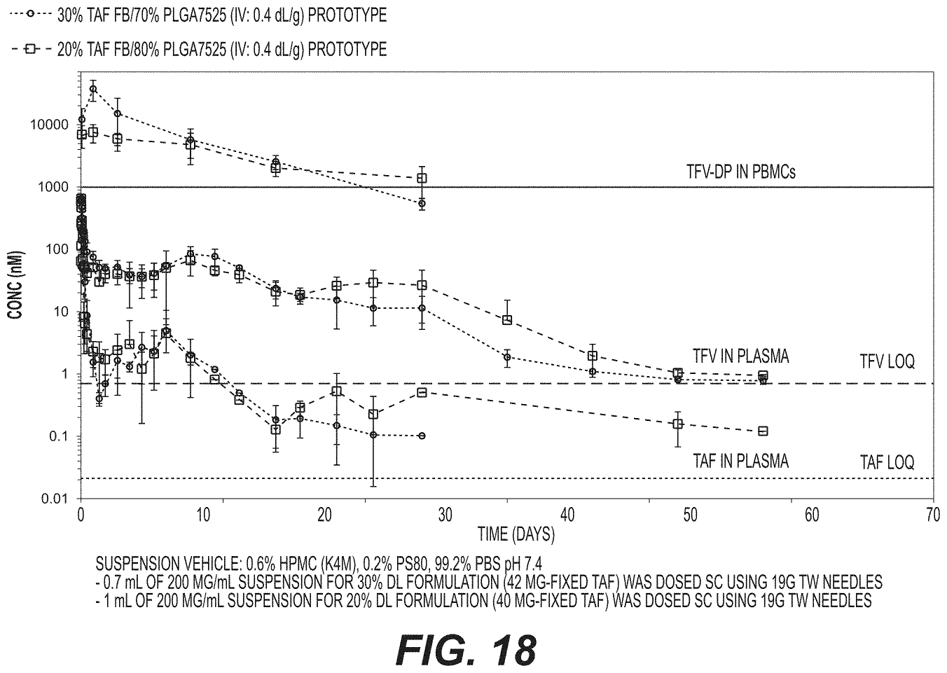

Two formulations containing 20% TAF/80% PLGA7525 and 30% TAF/70% PLGA7525 were evaluated in vitro (PBS, pH 7.4, 37° C.) for chemical stability and in dog model for Pharmacokinetic (PK) performance. The results from these experiments are summarized in FIG. 17 and FIG. 18. As seen in FIG. 17, the 30% TAF load formulation showed a better in vitro chemical stability of compared to 20% TAF load formulation. However, as seen in FIG. 18, the formulation containing 20% TAF load had lower initial in vivo burst and maintained a higher level of TFV-DP concentration (>1.3 μM) in PBMCs at day 28 compared to 30% TAF load formulation (0.5 μM). TAF concentration in plasma maintained for a longer time (about 56 days) for 20% TAF load formulation as compared to 30% TAF load formulation (about 28 days).

In another study, two formulations containing 20% TAF/80% PLGA8515 and 30% TAF/70% PLGA8515 were evaluated in dog model for Pharmacokinetic (PK) performance. 20% TAF load formulation had lower initial burst and maintained a higher level of TFV-DP concentration (>0.72 μM) in PBMCs at day 28 compared to 30% TAF load formulation (0.56 μM).

### Example 9. Process for Making Hot Melt Extruded Composition of TAF Drug Substance and Dexamethasone

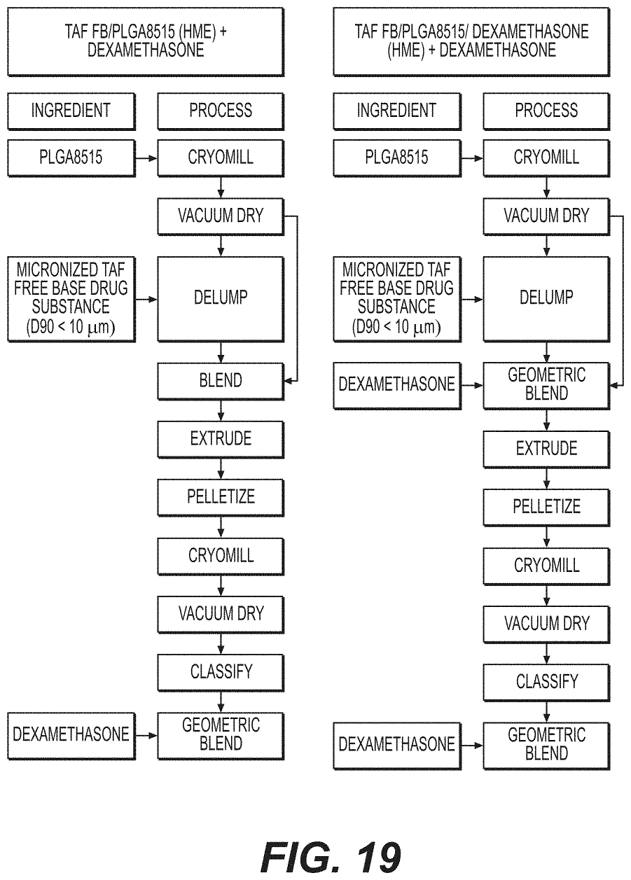

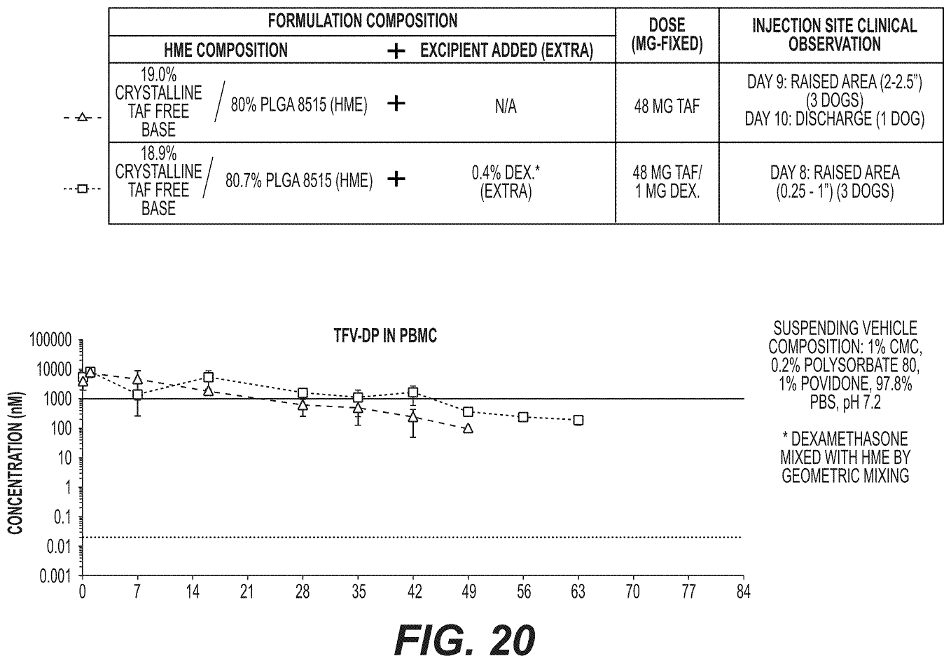

Dexamethasone was included in TAF PI formulation by (i) geometric mixing with TAF PI and/or (ii) including as component in hot melt extrusion process. Both these processes are summarized in FIG. 19. Two formulations 18.9% TAF/80.7% PLGA8515/0.4% dexamethasone and 19.8%/79.4%/0.8% TAF/PLGA8515/dexamethasone were prepared and evaluated in dog PK study. The results of these experiments are shown in FIG. 20. Clinical observation at injection site indicated smaller raised area (0.25-0.5″) for dexamethasone containing formulation as compared to 19% TAF/81% PLGA8515 formulation (2-2.5″). At day 42, the 18.9% TAF/80.7% PLGA8515/0.4% dexamethasone formulation showed a TFV-DP concentration in PBMCs of 1.3 μM while the 19% TAF/810% PLGA8515 formulation showed a TFV-DP concentration in PBMCs of 0.24 μM.

### Example 10. Manufacturing of the Suspending Vehicle Formulation

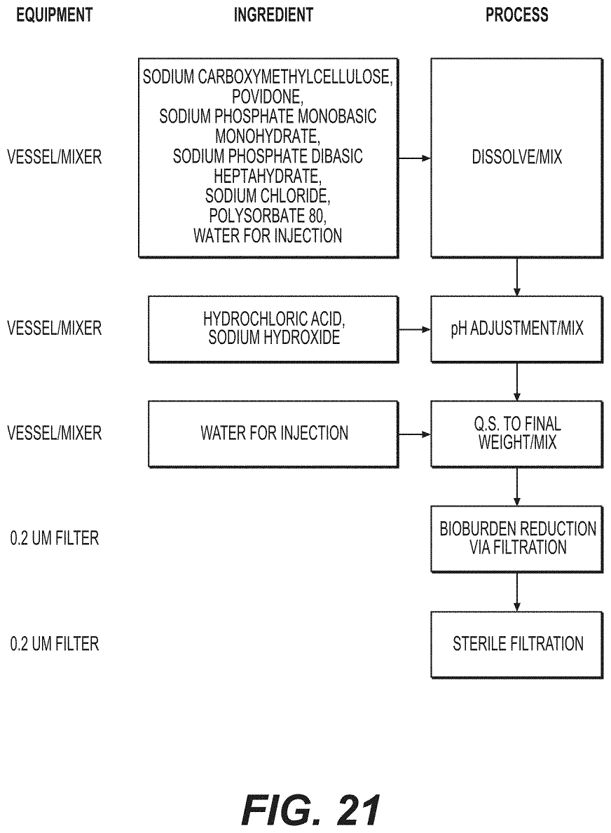

TAF PI (TAF powder for injection) suspending vehicle was manufactured through a series of unit process steps as depicted in the flow diagram of FIG. 21. Sodium carboxymethylcellulose, povidone, sodium phosphate dibasic heptahydrate, sodium phosphate monobasic monohydrate, sodium chloride, and polysorbate 80 were added into a portion of water for injection and mixed to dissolve. pH adjustment using HCl and/or NaOH was performed if needed, followed by addition of water for injection to achieve the final weight of bulk vehicle. The bulk solution was filtered through two in-line filters, a bioburden reduction filter followed by a sterilizing filter. The resulting sterile vehicle was filled into 6R Type I clear glass vials and fitted with coated rubber stoppers and aluminum seals with polypropylene flip-off caps.

Qualitative and Quantitative Composition of TAF PI
Suspending Vehicle

| | Composition | Unit Formula |
|---|---|---|
| Components | (% w/w) | (mg/unit) |
| Sodium | 1.000 | 30.90 |
| Carboxymethylcellulose | | |
| Povidone | 1.000 | 30.90 |
| Polysorbate 80 | 0.200 | 6.18 |
| Sodium Phosphate | 0.068 | 2.10 |
| Monobasic, Monohydrate | | |
| Sodium Phosphate, | 0.404 | 12.48 |
| Dibasic, Heptahydrate | | |
| Sodium Chloride | 0.699 | 21.60 |
| Hydrochloric Acid^{b} | as needed | as needed |
| Sodium Hydroxide^{b} | as needed | as needed |
| Water For Injection^{c} | q.s. | q.s. |
| Total | 100.000 | 3090.0 |

^{a}Represents amount for a deliverable volume of 3.0 mL. A total volume of 4.0 mL was filled into each vial to ensure a deliverable volume of 3.0 mL.
^{b}Hydrochloric acid and sodium hydroxide were used to adjust the pH during manufacturing.
^{c}The total quantity of water for injection was adjusted to maintain the target total amount.

### Example 11. Selection of a Suspending Agent Viscosity Modifier

Syringeability/injectability of various vehicle compositions was evaluated. The various suspending vehicles evaluated are summarized in the table below. These suspending vehicles were prepared analogous to the procedure described in Example 10. The suspensions at 250 mg/mL solid concentration were prepared with adding suspending vehicle to TAF PI.

| Suspending | |
|---|---|
| Vehicle No. | Suspending Vehicle |
| 1 | 0.6% HPMC, 0.2% Tween, 99.2% PBS pH 7.4 |
| 2 | 1.0% HPMC, 0.2% Tween, 98.8% PBS pH 7.4 |
| 3 | 0.6% HPMC, 0.2% Tween 80, 1% Kollidon K12, 98.2% |
| | PBS pH 7.4 |
| 4 | 1% HPMC, 0.2% Tween 80, 1% Kollidon K12, 97.8% |
| | PBS pH 7.4 |
| 5 | 0.6% CMC, 0.2% Tween, 99.2% PBS pH 7.4 |
| 6 | 1.0% CMC, 0.2% Tween, 98.8% PBS pH 7.4 |
| 7 | 1.0% CMC, 0.4% Tween, 98.6% PBS pH 7.4 |
| 8 | 1.0% CMC, 0.2% Tween 80, 1% Kollidon K12, 97.8% |
| | PBS pH 7.4 |
| 9 | 1.0% CMC, 0.4% Tween, 1% Kollidon K12, 97.6% |
| | PBS pH 7.4 |
| 10 | 1.0% CMC, 0.4% Tween, 2% Kollidon K12, 96.6% |
| | PBS pH 7.4 |
| 11 | 0.2% Tween 80, 5% Kollidon K12, 94.8% PBS pH 7.4 |
| 12 | 1% Tween 80, 5% Kollidon K12, 94.0% PBS pH 7.4 |

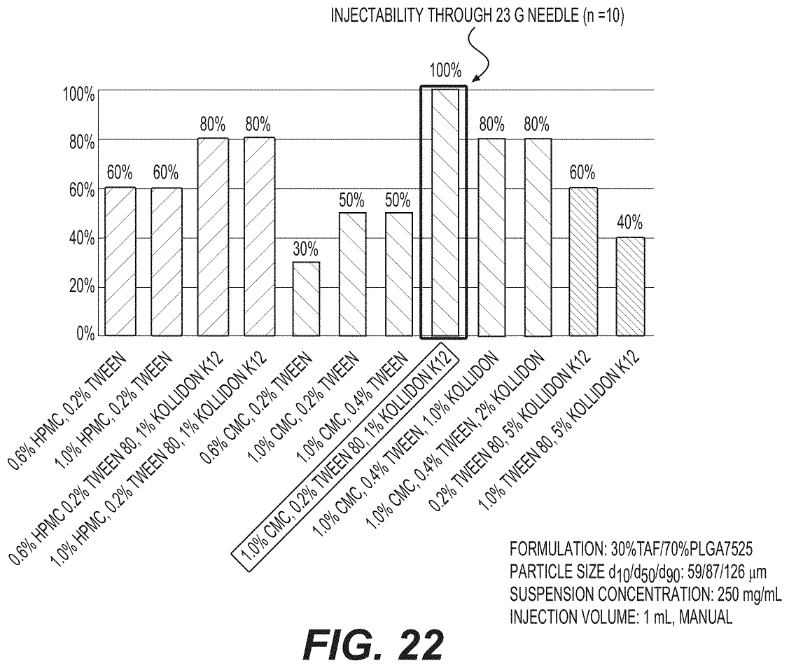

To evaluate syringeability/injectability, a 30% crystalline TAF free base (Form I) 70% PLGA 7525 formulation (with a d₉₀ of 126 μm) was suspended in the vehicle to obtain a 250 mg/mL suspension. Generally, TAF PI was reconstituted with suspending vehicle and mixed by vortexing. During each experiment, 1 mL of each sample was injected through a 23 G needle (n=10) by hand. The percentage of each sample that passed successfully through the needle without clogging was recorded. The results of these experiments are summarized in FIG. 22.

Statistically, there was no difference in injectability between the 1.0% HPMC/0.2% Tween 80 and 1.0% CMC/0.2% Tween 80 vehicles without Povidone K12 (suspending vehicle nos. 2 and 6). With these suspending vehicles, 60% and 50% of samples passed through the 23 G needle without clogging, respectively. When 1.0% of Povidone K12 was added (suspending vehicle nos. 4 and 8), there was still no statistical difference between the HPMC and CMC formulations, but the injectability increased. With the 1.0% HPMC/0.2% Tween 80/1.0% Povidone K12 vehicle (suspending vehicle no. 4), 80% of samples passed through at 23 G needle without clogging, and with the 1.0% CMC/0.2% Tween 80/1.0% Povidone K12 vehicles (suspending vehicle no. 8), 100% of samples passed.

Vehicle formulations with Povidone K12, but without CMC or HPMC did not improve injectability. With suspension samples containing 0.2% Tween 80 and 5% Povidone K12 (suspending vehicle no. 11) and 1.0% Tween 80 and 5% Povidone K12 (suspending vehicle no. 12), only 60 and 40% of samples passed through at 23 G needle without clogging, respectively.

As compared to HPMC formulations, CMC formulations were had easier manufacturability.

Amongst the various suspending vehicles evaluated, 1.0% CMC, 0.2% Tween 80, 1% Kollidon K12, and 97.8% PBS pH 7.4 resulted in best syringeability/injectability (100% of the samples passed successfully through the 23 G needle without any clogging)

### Example 12. Selection of a Wetting Agent

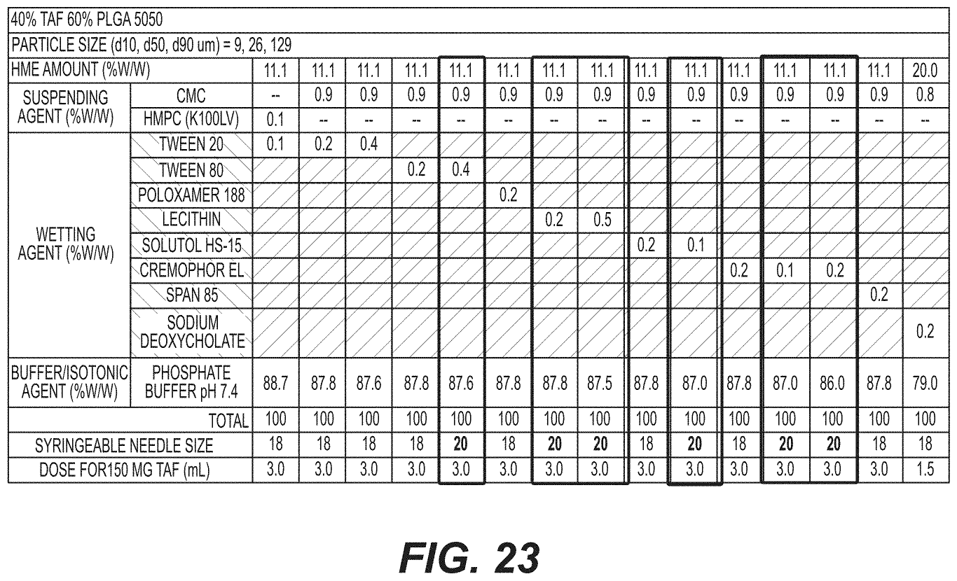

Suspending vehicles with various wetting agents were prepared. The wetting agents evaluated were (i) Tween 20, (ii) Tween 80, (iii) Poloxamer 188, (iv) lecithin, (v) Solutol HS-15, (vi) cremophor EL, (vii) span 85, and (viii) sodium deoxycholate. A formulation 40% TAF (free base, crystalline, and micronized) and 60% PLGA 5050 was used to obtain a 125 mg/mL suspension. These suspensions were evaluated for syringeability/injectability. The results of these experiments are summarized in FIG. 23. As seen, Tween 80 and lecithin improved syringeability/injectability through a 20 G needle when added in minimal amounts. Additionally, Tween 80 could accommodate sterilization via filtration.

### Example 13. Comparison of TAF Sebacate Formulations

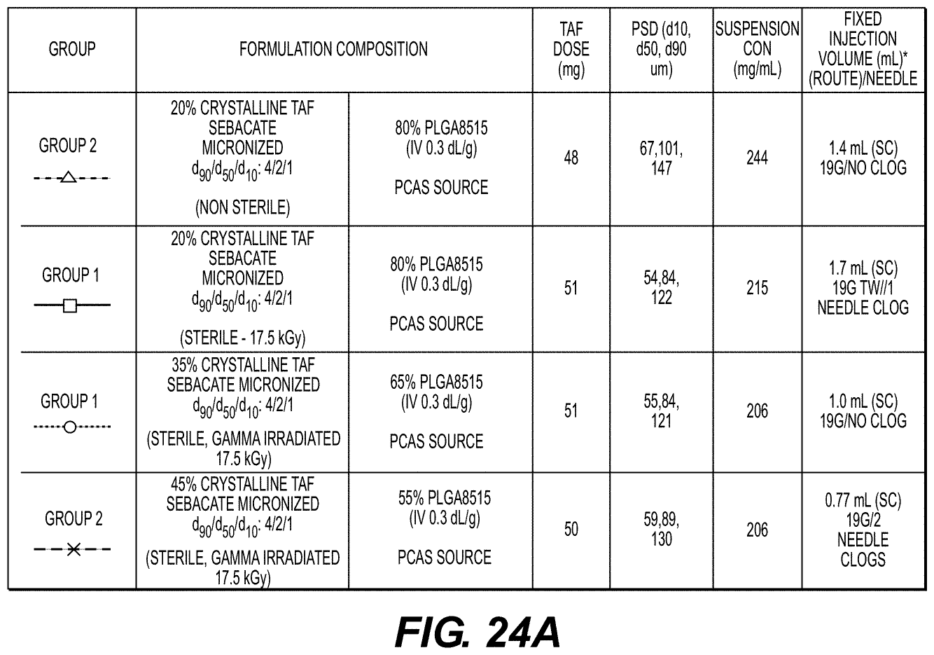

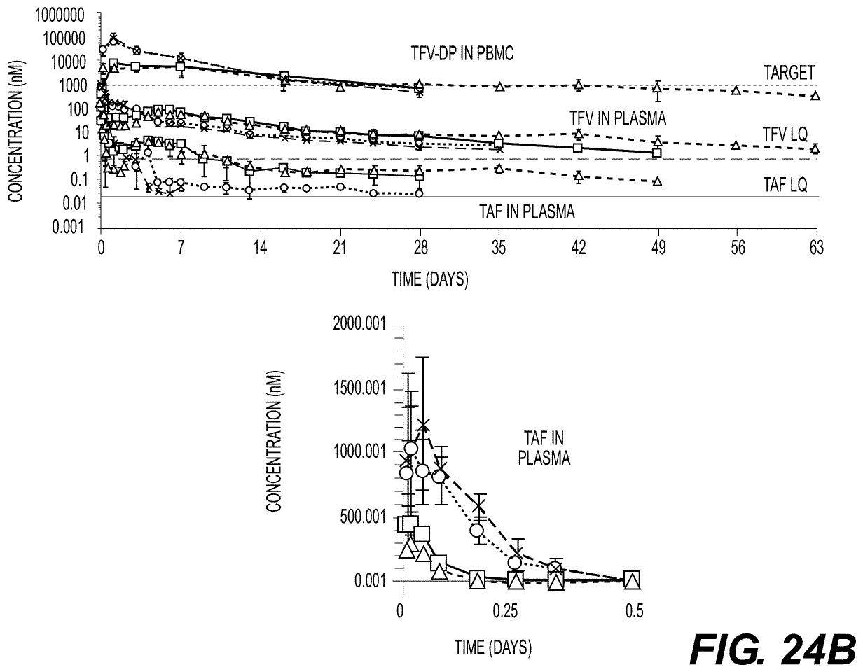

Following formulations were prepared and evaluated in dog model for PK performance. The results of these experiments are shown in FIGS. 24A and 24B. As seen, TAF and TFV concentration in plasma and TFV-DP concentration in PBMCs is higher for 20% TAF sebacate formulations than for 35 and 45% TAF sebacate formulations.

| Formulation | |
|---|---|
| No. | Composition |
| I | 20% crystalline TAF Sebacate (Form I) micronized, d₉₀/d₅₀/d₁₀: 4/2/1 (non |
| | sterile) and 80% PLGA8515 (IV 0.3 dL/g) |
| II | 20% crystalline TAF Sebacate (Form I) micronized, d₉₀/d₅₀/d₁₀: 4/2/1, |
| | (sterile-17.5 kGy) and 80% PLGA8515 (IV 0.3 dL/g) |
| III | 35% crystalline TAF Sebacate (Form I) micronized, d₉₀/d₅₀/d₁₀: 4/2/1, |
| | (sterile, gamma irradiated 17.5 kGy) and 65% PLGA8515 (IV 0.3 dL/g) |
| IV | 45% Crystalline TAF Sebacate (Form I) micronized, d₉₀/d₅₀/d₁₀: 4/2/1, |
| | (sterile, gamma irradiated 17.5 kGy) and 55% PLGA8515 (IV 0.3 dL/g) |

The clinical observations from these studies are summarized below. As seen, clinical observations at injection site for all formulations indicated soft to firm raised areas (1-7.6 cm) that did not dissipate 13 days after dosing.

| Formulation | Clinical observations | | |
|---|---|---|---|
| No. | Day 7 | Day 8/9 | Day 13 |
| I | Slight raised area | Firm slight raised | Soft to Firm slight |
| | (2.5 cm diameter) | area (1.3-3.8 cm | raised area |
| | (1 dog) | diameter) | (2.5-7.6 cm |
| | | (2 dogs) | diameter) (2 dogs), |
| | | | Scab (1 dog) |
| II | Firm raised area | Firm raised area | Semi-firm lump |
| | (3-6 cm diameter, | (4-6 cm diameter, | (~1 cm diameter), |
| | 2 cm height) | 2 cm height) | no rupture but pain |
| | (3 dogs), | (3 dogs) | when palpated |
| | dose site ruptured, | | (1 dog) |
| | discharge clear | | |
| | (1 dog) | | |
| III | Raised area | Soft raised area | Semi-firm to Firm |
| | (1 cm height, | (0.5-1.5 cm height, | raised area |
| | 4-6 cm diameter) | 2-3 cm length, | 1-1.5 cm height, 1- |
| | (3 dogs) | 2-3 cm width) | 2 cm length, 1-2 |
| | | (3 dogs) | cm width) |
| | | | (2 dogs) |
| IV | Raised area | Soft to Firm raised | Firm raised area |
| | (0.5-1 cm height, | area (1-2 cm height, | (1 cm height, 2 cm |
| | 4-5 cm length, | 2-3 cm length, | length, 3 cm width) |
| | 2 cm width) | 3-4 cm width) | (1 dog) |
| | (2 dogs) | (2 dogs) | |

### Example 14. Use of Additional Therapeutic Agents

Following formulations were prepared and evaluated in dog model.

| No. | Formulation Composition | | |
|---|---|---|---|
| I | 20% Crystalline TAF | 80% PLGA8515 | |
| | Sebacate Micronized | (IV 0.3 dL/g) | |
| | d₉₀/d₅₀/d₁₀: 4/2/1 | | |
| | (non sterile) | | |
| II | 20% Crystalline TAF | 80% PLGA8515 | |
| | Sebacate Micronized | (IV 0.3 dL/g) | |
| | d₉₀/d₅₀/d₁₀: 4/2/1 | | |
| | (Sterile-17.5 kGy) | | |
| III | 19.7% Crystalline | 78.9% | 1.4% Methylprednisolone |
| | TAF Sebacate | PLGA8515 | acetate |
| | Micronized | (IV 0.3 dL/g) | |
| | d₉₀/d₅₀/d₁₀: 4/2/1 | | |
| | (Sterile-17.5 kGy) | | |
| IV | 35% Crystalline TAF | 65% PLGA8515 | |
| | Sebacate Micronized | (IV 0.3 dL/g) | |
| | d₉₀/d₅₀/d₁₀: 4/2/1 | | |
| V | 34.0% Crystalline TAF | 63.1% | 2.9% Methylprednisolone |
| | Sebacate Micronized | PLGA8515 | acetate |
| | | (IV 0.3 dL/g) | |
| VI | 34.5% Crystalline TAF | 64.0% | 1.5% Methylprednisolone |
| | Sebacate Micronized | PLGA8515 | acetate |
| | | (IV 0.3 dL/g) | |
| VII | 19% Crystalline TAF | 81% PLGA8515 | |
| | free base Micronized | (IV 0.3 dL/g) | |
| | d₉₀/d₅₀/d₁₀: 4/2/1 | | |
| | (Sterile-17.5kGy) | | |
| VIII | 18.5% Crystalline TAF | 79.1% | 2.4% Methylprednisolone |
| | free base Micronized | PLGA8515 | acetate |
| | d₉₀/d₅₀/d₁₀: 4/2/1 | (IV 0.3 dL/g) | |
| | (Sterile-17.5kGy) | | |

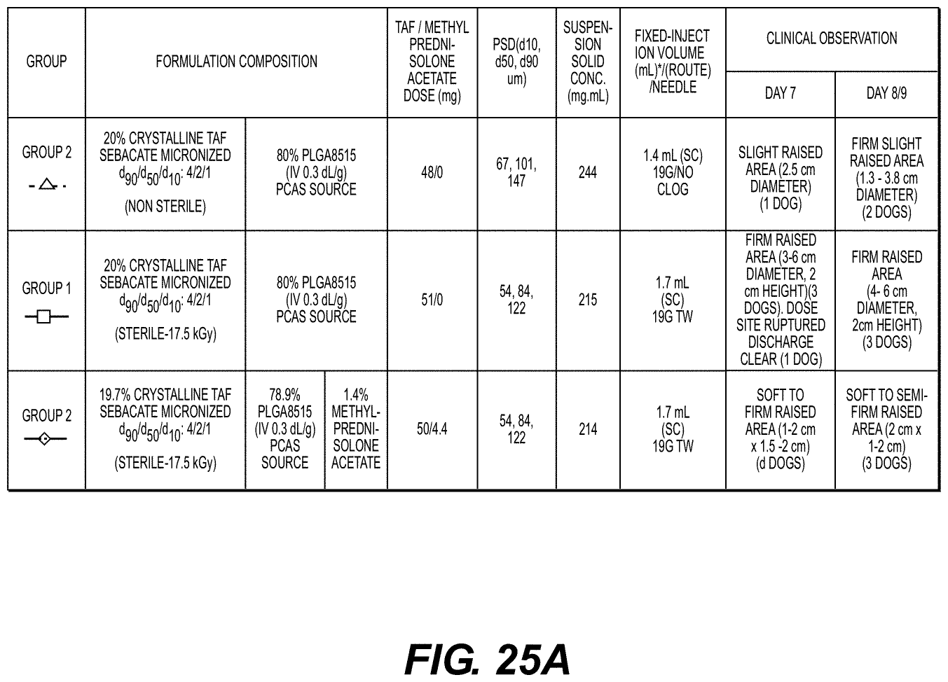

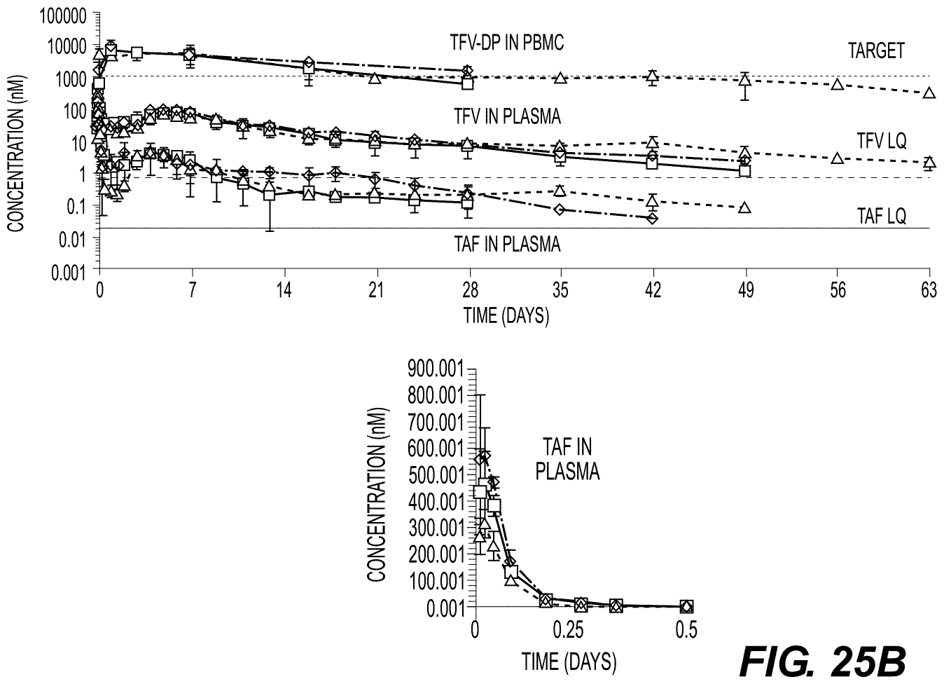

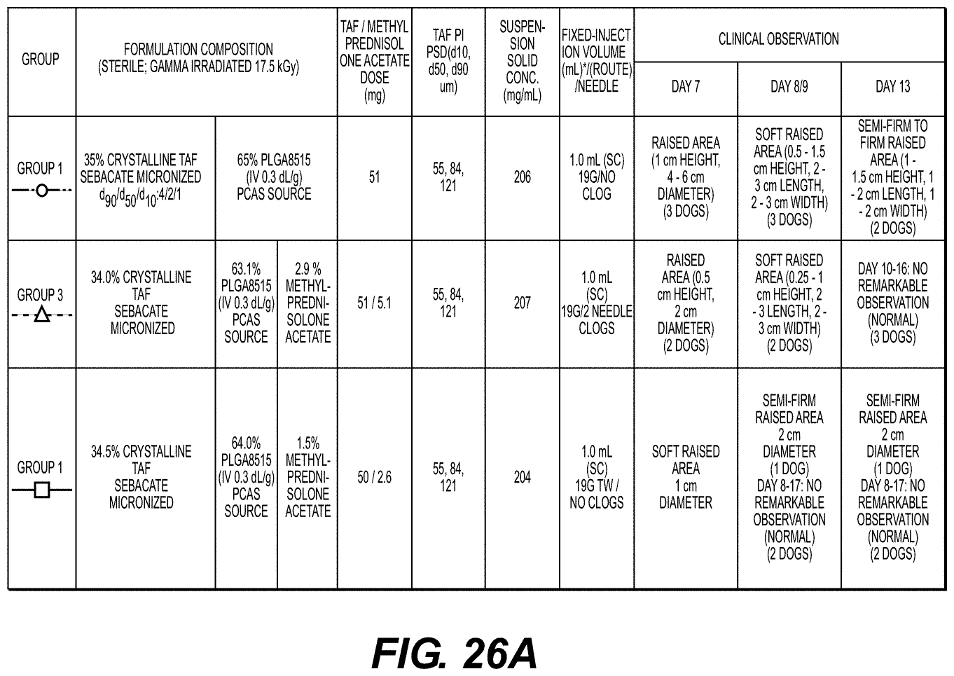

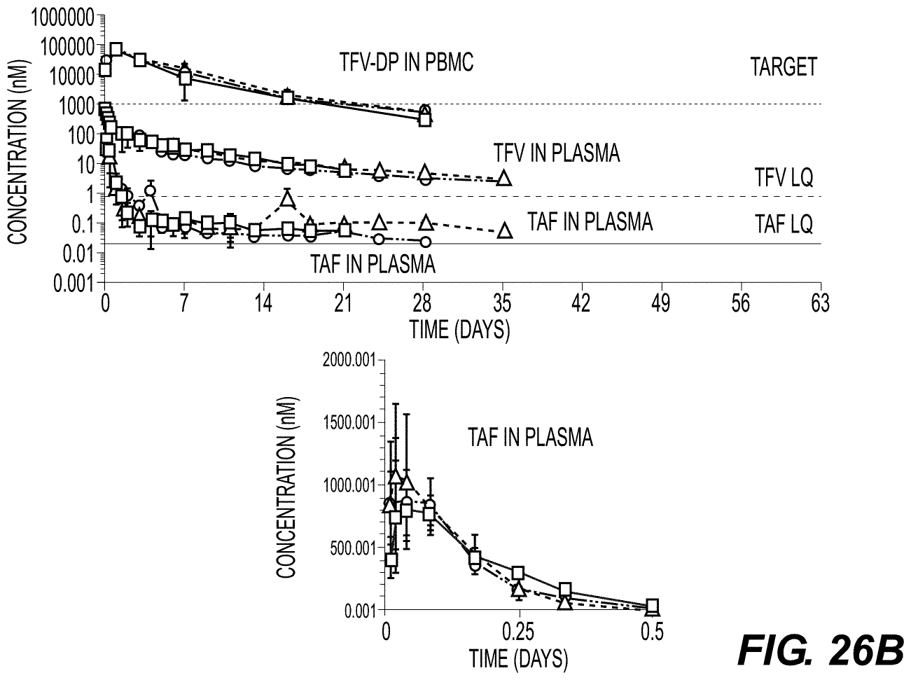

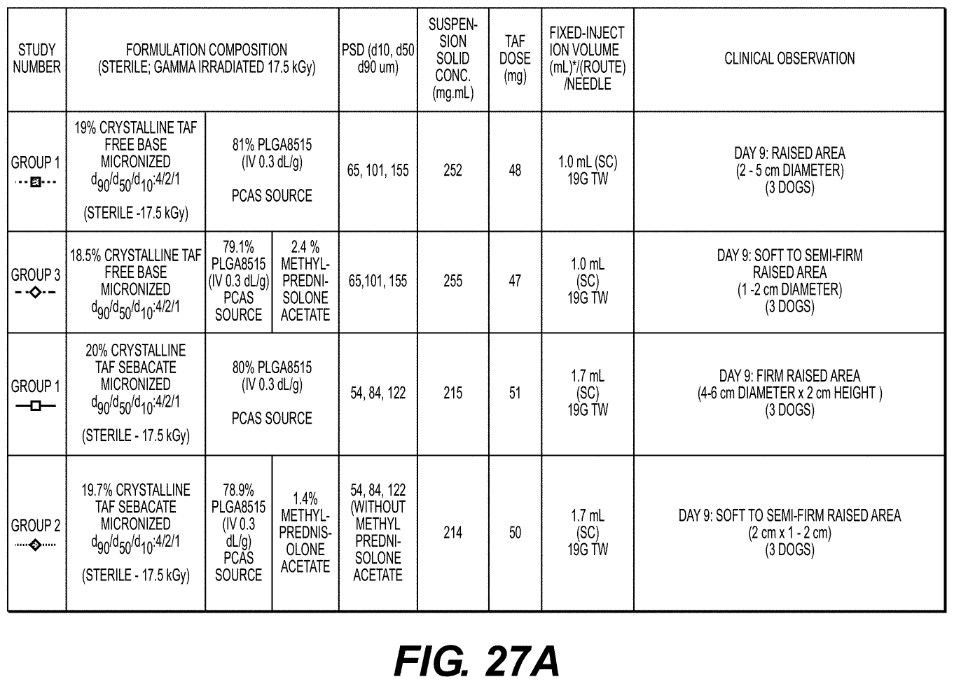

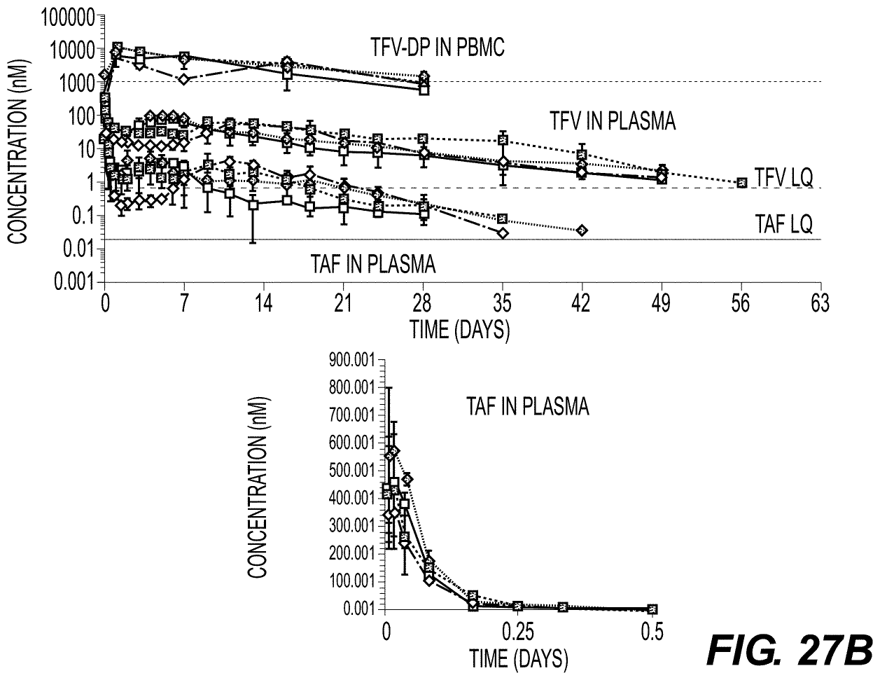

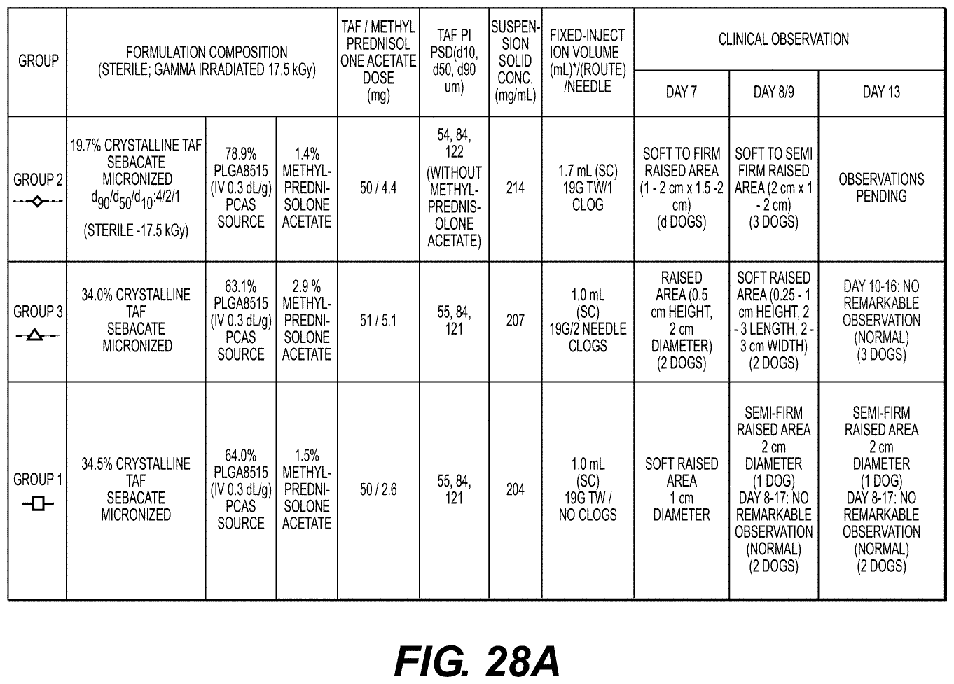

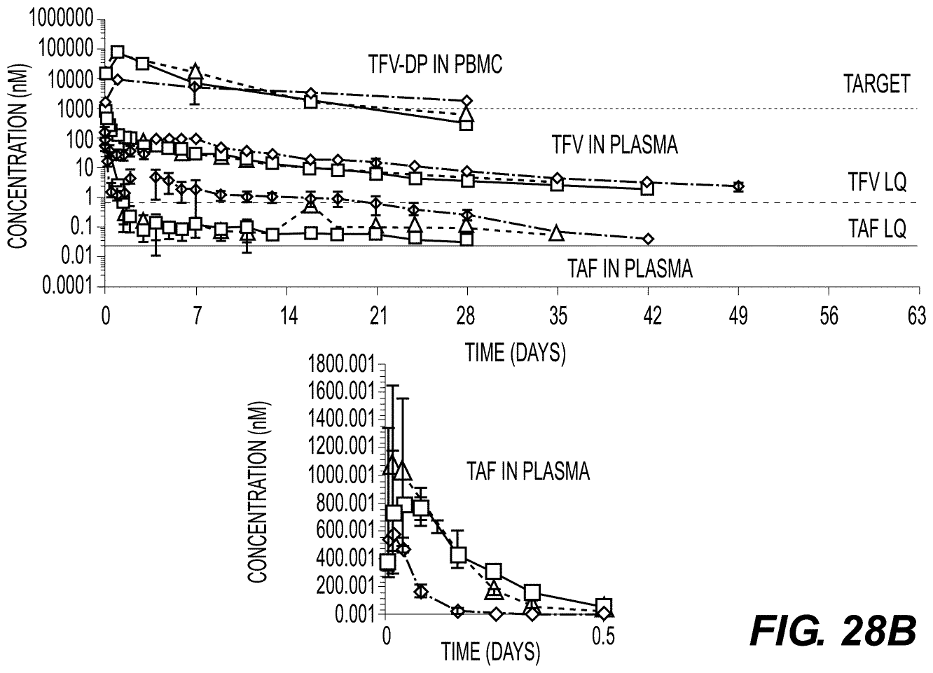

The results of these experiments and the clinical observations are summarized in FIGS. 25-27. As seen, in FIGS. 25-27, clinical observations at injection site generally indicated smaller and/or softer raised area with methylprednisolone acetate as compared to formulations without it. Further, as seen in FIGS. 28A and 28B, amongst formulations III, V, and VI above, formulation III shows reduced initial burst and increased TAF/TFV concentration in plasma.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter, and is not intended to limit the appended claims to the specific embodiments illustrated.

What is claimed:

1. A pharmaceutical composition comprising:
(i) a compound of Formula I: 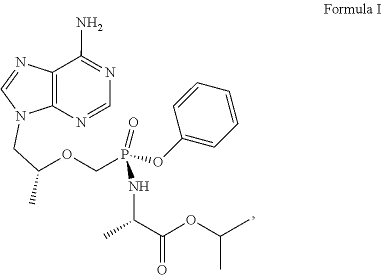 [SMILES: CC(C)OC(=O)[C@H](C)N[P@](=O)(CO[C@H](C)Cn1cnc2c(N)ncnc21)Oc1ccccc1]
or a pharmaceutically acceptable salt thereof, and
(ii) a biodegradable polymer.

2. The pharmaceutical composition of claim 1, wherein the compound of Formula I, or the pharmaceutically acceptable salt thereof, and the biodegradable polymer together constitute 99-100% of the composition weight.

3. The pharmaceutical composition of claim 1, wherein the compound of Formula I, or the pharmaceutically acceptable salt thereof, and the biodegradable polymer together constitute 99.5-100% of the composition weight.

4. The pharmaceutical composition of any one of claims 1-3, wherein the pharmaceutical composition consists essentially of the compound of Formula I, or the pharmaceutically acceptable salt thereof, and the biodegradable polymer.

5. The pharmaceutical composition of any one of claims 1-4, wherein the pharmaceutical composition consists of the compound of Formula I, or the pharmaceutically acceptable salt thereof, and the biodegradable polymer.

6. The pharmaceutical composition of claim 1, further comprising an additional agent.

7. The pharmaceutical composition of claim 6, wherein the additional agent is a therapeutic agent.

8. The pharmaceutical composition of claim 6 or 7, wherein the additional agent is an anti-inflammatory agent.

9. The pharmaceutical composition of claim 6 or 7, wherein the additional agent is a steroid.

10. The pharmaceutical composition of any one of claims 6-8, wherein the additional agent is and a corticosteroid.

11. The pharmaceutical composition of any one of claims 6-10, wherein the additional agent is dexamethasone.

12. The pharmaceutical composition of any one of claims 6-11, wherein (i) the compound of Formula I, or the pharmaceutically acceptable salt thereof, (ii) the biodegradable polymer, and (iii) the additional agent together constitute 99-100% of the pharmaceutical composition weight.

13. The pharmaceutical composition of any one of claims 6-12, wherein (i) compound of Formula I, or the pharmaceutically acceptable salt thereof, (ii) the biodegradable polymer, and (iii) the additional agent together constitute 99.5-100% of the pharmaceutical composition weight.

14. The pharmaceutical composition of any one of claims 6-13, wherein the pharmaceutical composition consists essentially of (i) the compound of Formula I, or the pharmaceutically acceptable salt thereof, (ii) the biodegradable polymer, and (iii) the additional agent.

15. The pharmaceutical composition of any one of claims 6-14, wherein the pharmaceutical composition consists of (i) the compound of Formula I, or the pharmaceutically acceptable salt thereof, (ii) the biodegradable polymer, and (iii) the additional agent.

16. The pharmaceutical composition of any one of claims 1-15, wherein the pharmaceutical composition does not comprise sucrose acetate isobutyrate.

17. The pharmaceutical composition of any one of claims 1-16, wherein the compound of Formula I, or the pharmaceutically acceptable salt thereof is selected from the group consisting of tenofovir alafenamide free base, tenofovir alafenamide hemipamoate, tenofovir alafenamide sebacate, tenofovir alafenamide napsylate, tenofovir alafenamide orotate, tenofovir alafenamide vanillate, and tenofovir alafenamide bis-xinafoate.

18. The pharmaceutical composition of any one of claims 1-17, wherein the compound of Formula I, or the pharmaceutically acceptable salt thereof is selected from the group consisting of tenofovir alafenamide free base, tenofovir alafenamide orotate, tenofovir alafenamide vanillate, tenofovir alafenamide sebacate, or tenofovir alafenamide bis-xinafoate.

19. The pharmaceutical composition of any one of claims 1-18, wherein the pharmaceutical composition comprises the pharmaceutically acceptable salt of the compound of Formula I.

20. The pharmaceutical composition of any one of claims 1-19, wherein the pharmaceutically acceptable salt is a orotate salt, a vanillate salt, a sebacate salt, or a bis-xinafoate salt.

21. The pharmaceutical composition of any one of claims 1-20, wherein the pharmaceutically acceptable salt is a sebacate salt.

22. The pharmaceutical composition of any one of claims 1-18, wherein the pharmaceutical composition comprises free base of the compound of Formula I.

23. The pharmaceutical composition of any one of claims 1-22, wherein the compound of Formula I, or the pharmaceutically acceptable salt thereof, is crystalline.

24. The pharmaceutical composition of claim 23, wherein the compound of Formula I, or the pharmaceutically acceptable salt thereof, is selected from the group consisting of crystalline tenofovir alafenamide free base, crystalline tenofovir alafenamide orotate, crystalline tenofovir alafenamide vanillate, crystalline tenofovir alafenamide sebacate, and crystalline tenofovir alafenamide bis-xinafoate.

25. The pharmaceutical composition of claim 23 or 24, wherein the pharmaceutical composition comprises a crystalline form of tenofovir alafenamide free base.

26. The pharmaceutical composition of claim 25, wherein the crystalline form of tenofovir alafenamide free base is characterized by an XRPD pattern comprising degree 2θ-reflections (+/−0.2 degrees 2θ) at 11.3°, 19.6°, and 22.4°.

27. The pharmaceutical composition of claim 25 or 26, wherein the crystalline form of tenofovir alafenamide free base is characterized by an XRPD pattern comprising degree 2θ-reflections (+/−0.2 degrees 2θ) at 7.4°, 11.3°, 19.6°, 21.3° and 22.4°.

28. The pharmaceutical composition of claim 23 or 24, wherein the pharmaceutical composition comprises a crystalline form of TAF sebacate.

29. The pharmaceutical composition of any one of claims 23, 24 and 28, wherein the pharmaceutical composition comprises crystalline Form I of TAF sebacate.

30. The pharmaceutical composition of claim 29, wherein the crystalline Form I of TAF sebacate is characterized by an XRPD pattern comprising degree 2θ-reflections (+/−0.2 degrees 2θ) at 6.6°, 9.4°, and 9.6°.

31. The pharmaceutical composition of claim 29 or 30, wherein the pharmaceutical composition comprises crystalline form of TAF sebacate is characterized by an XRPD pattern comprising degree 2θ-reflections (+/−0.2 degrees 2θ) at 5.3°, 6.6°, 9.4°, 9.6°, and 19.80.

32. The pharmaceutical composition of any one of claims 1-31, wherein the compound of Formula I, or the pharmaceutically acceptable salt thereof, is in a micronized form.

33. The pharmaceutical composition of claim 32, wherein the micronized form has a d₉₀ of ≤about 10 μm.

34. The pharmaceutical composition of claim 32 or 33, wherein the micronized form has a d₉₀ of about 1-10 μm.

35. The pharmaceutical composition of any one of claims 32-34, wherein the micronized form has a d₉₀ of about 1-5 μm.

36. The pharmaceutical composition of any one of claims 32-35, wherein the micronized form has a d₉₀ of about 4 μm.

37. The pharmaceutical composition of any one of claims 32-36, wherein the micronized form has a d₅₀ of about 1-10 μm.

38. The pharmaceutical composition of any one of claims 32-37, wherein the micronized form has a d₅₀ of about 1-5 μm.

39. The pharmaceutical composition of any one of claims 32-38, wherein the micronized form has a d₅₀ of about 2 μm.

40. The pharmaceutical composition of any one of claims 32-39, wherein the micronized form has a d₁₀ of about 0.1-10 μm.

41. The pharmaceutical composition of any one of claims 32-40, wherein the micronized form has a d₁₀ of about 0.5-5 μm.

42. The pharmaceutical composition of claim any one of claims 32-41, wherein the micronized form has a d₁₀ of about 1 μm.

43. The pharmaceutical composition of any one of claims 32-42, wherein the micronized form has a d₉₀ of about 1-10 μm, a d₅₀ of about 1-5 μm, and a d₁₀ of about 0.1-2 μm.

44. The pharmaceutical composition of any one of claims 32-43, wherein the micronized form has a d₉₀ of about 4 μm, a d₅₀ of about 2 μm, and a d₁₀ of about 1 μm.

45. The pharmaceutical composition of any one of claims 1-44, wherein the compound of Formula I, or the pharmaceutically acceptable salt thereof, is present in an amount of about 15-45% w/w of the pharmaceutical composition weight.

46. The pharmaceutical composition of any one of claims 1-45, wherein the compound of Formula I, or the pharmaceutically acceptable salt thereof, is present in an amount of about 15-35% w/w of the pharmaceutical composition weight.

47. The pharmaceutical composition of any one of claims 1-46, wherein the compound of Formula I, or the pharmaceutically acceptable salt thereof, is present in an amount of about 29-31% w/w the pharmaceutical composition weight.

48. The pharmaceutical composition of any one of claims 1-47, wherein the compound of Formula I, or the pharmaceutically acceptable salt thereof, is present in an amount of about 30% w/w of the pharmaceutical composition weight.

49. The pharmaceutical composition of any one of claims 1-46, wherein the compound of Formula I, or the pharmaceutically acceptable salt thereof, is present in an amount of about 18-20% w/w of the pharmaceutical composition weight.

50. The pharmaceutical composition of any one of claims 1-46 and 49, wherein the compound of Formula I, or the pharmaceutically acceptable salt thereof, is present in an amount of about 19% w/w of the pharmaceutical composition weight.

51. The pharmaceutical composition of any one of claims 1-50, wherein the biodegradable polymer is present in an amount of about 55-85% w/w of the pharmaceutical composition weight.

52. The pharmaceutical composition of any one of claims 1-51, wherein the biodegradable polymer is present in an amount of about 65-85% w/w of the pharmaceutical composition weight.

53. The pharmaceutical composition of any one of claims 1-52, wherein the biodegradable polymer is present in an amount of 69-71% w/w of the pharmaceutical composition weight.

54. The pharmaceutical composition of any one of claims 1-53, wherein the biodegradable polymer is present in an amount of 70% w/w of the pharmaceutical composition weight.

55. The pharmaceutical composition of any one of claims 1-52, wherein the biodegradable polymer is present in an amount of about 80-82% w/w of the pharmaceutical composition weight.

56. The pharmaceutical composition of any one of claims 1-52 and 55, wherein the biodegradable polymer is present in an amount of about 810% w/w of the pharmaceutical composition weight.

57. The pharmaceutical composition of any one of claims 1-56, wherein the biodegradable polymer is PLGA (poly(lactic-co-glycolic acid)).

58. The pharmaceutical composition of claim 57, wherein the PLGA is PLGA7525 (about 75% lactic acid and about 25% glycolic acid).

59. The pharmaceutical composition of claim 57, wherein the PLGA is PLGA 8525 (about 85% lactic acid and about 15% glycolic acid).

60. The pharmaceutical composition of any one of claims 1-59, wherein the composition is a micronized composition.

61. The pharmaceutical composition of any one of claim 60, wherein the micronized composition has a d₁₀ value of about 35-80 μm.

62. The pharmaceutical composition of claim 60 or 61, wherein the micronized composition has a d₁₀ value of greater than about 50 μm.

63. The pharmaceutical composition of any one of claims 60-62, wherein the micronized composition has a d₁₀ value of about 50-70 μm.

64. The pharmaceutical composition of any one of claims 60-63, wherein the micronized composition has a d₁₀ value of about 58-62 μm.

65. The pharmaceutical composition of any one of claims 60-64, wherein the micronized composition has a d₁₀ value of about 60 μm.

66. The pharmaceutical composition of any one of claims 60-65, wherein the micronized composition has a d₉₀ value of less than about 250 μm.

67. The pharmaceutical composition of any one of claims 60-66, wherein the micronized composition has a d₉₀ value of about 120-200 μm.

68. The pharmaceutical composition of any one of claims 60-67, wherein the micronized composition has a d₉₀ value of about 130-160 μm.

69. The pharmaceutical composition of any one of claims 60-68, wherein the micronized composition has a d₉₀ value of about 150-160 μm.

70. The pharmaceutical composition of any one of claims 60-69, wherein the micronized composition has a d₅₀ value of about 80-100 μm.

71. The pharmaceutical composition of any one of claims 60-69, wherein the micronized composition has a d₅₀ value of about 88-92 μm.

72. The pharmaceutical composition of any one of claims 60-71, wherein the micronized composition has a d₅₀ value of about 90 μm.

73. The pharmaceutical composition of any one of claims 60-72, wherein the micronized composition has a d₉₀ value of about 100-150 μm, a d₅₀ value of about 80-150 μm, and a d₁₀ value of about 35-80 μm.

74. The pharmaceutical composition of any one of claims 60-73, wherein the micronized composition has a d₉₀ value of about 120-140 μm, a d₅₀ value of about 80-100 μm, and a d₁₀ value of about 50-70 μm.

75. The pharmaceutical composition of any one of claims 60-74, wherein the micronized composition has a d₉₀ value of about 132 μm, a d₅₀ value of about 90 μm, and a d₁₀ value of about 60 μm.

76. The pharmaceutical composition of any one of claims 1-75, wherein storage for about one month at a temperature of about 30° C. and a relative humidity of 75% results in less than 0.5% (w/w) impurities, wherein the impurities comprises PMPA, PMPA anhydride, monophenyl PMPA, PMPA monoamidite, and/or phenol.

77. The pharmaceutical composition of any one of claims 1-76, wherein storage for about one month at a temperature of about 30° C. and a relative humidity of 75% results in about 0.25% to about 0.45% (w/w) impurities, wherein the impurities comprises PMPA, PMPA anhydride, monophenyl PMPA, PMPA monoamidite, and/or phenol.

78. The pharmaceutical composition of any one of claims 1-77, prepared by hot melt extrusion.

79. A pharmaceutical formulation comprising the pharmaceutical composition of any one of claims 1-78 and a suspending vehicle.

80. The pharmaceutical formulation of claim 79, wherein the suspending vehicle comprising (i) a suspending agent, (ii) a wetting agent, and (iii) a buffer.

81. The pharmaceutical formulation of claim 79 or 80, wherein the suspending agent is selected from the group consisting of carboxy methyl cellulose, hydroxypropyl methylcellulose, and povidone K12.

82. The pharmaceutical formulation of claim 80 or 81, wherein the suspending agent is carboxy methyl cellulose or povidone K12.

83. The pharmaceutical formulation of any one of claims 80-82, wherein the wetting agent is selected from the group consisting of Tween 20, Tween 80, poloxamer 188, Lecithin, Solutol HS-15, Cremophor EL, Span 85, and sodium deoxycholate.

84. The pharmaceutical formulation of any one of claims 80-83, wherein the wetting agent is Tween 80.

85. The pharmaceutical formulation of any one of claims 80-84, wherein the buffer is a phosphate buffer.

86. The pharmaceutical formulation of any one of claims 80-85, wherein the buffer comprises sodium phosphate monobasic, sodium phosphate dibasic, or sodium chloride.

87. The pharmaceutical formulation of any one of claims 80-86, wherein the suspending vehicle further comprises water for injection.

88. The pharmaceutical formulation of any one of claims 80-87, wherein the suspending vehicle comprises:
(i) carboxy methyl cellulose in an amount of about 1% w/w of the suspending vehicle weight,
(ii) Tween 80 in an amount of about 0.2% w/w of the suspending vehicle weight,
(iii) Povidone K12 in an amount of about 1% w/w of the suspending vehicle weight, and
(iv) PBS in an amount of about 97.8% w/w of the suspending vehicle weight.

89. The pharmaceutical formulation of any one of claims 80-88, wherein the suspending vehicle has a pH of about 7.0-7.5.

90. The pharmaceutical formulation of any one of claims 80-89, wherein the suspending vehicle has a pH of about 7.4.

91. The pharmaceutical formulation of any one of claims 79-90, wherein the compound of Formula I, or the pharmaceutically acceptable salt thereof, is present at a concentration of about 45-50 mg/mL.

92. The pharmaceutical formulation of any one of claims 79-91, wherein the compound of Formula I, or the pharmaceutically acceptable salt thereof, is present at a concentration of about 48 mg/mL.

93. The pharmaceutical formulation of any one of claims 79-92, wherein the pharmaceutical formulation is a suspension.

94. The pharmaceutical formulation of any one of claims 79-93, wherein the pharmaceutical formulation is for administration by injection.

95. The pharmaceutical formulation of any one of claims 79-94, wherein the pharmaceutical formulation is for subcutaneous injection.

96. The pharmaceutical formulation of any one of claims 79-95, wherein after administration to a human subject, a PMBC of the human subject has a tenofovir-diphosphate concentration of greater than 1 μM.

97. The pharmaceutical formulation of any one of claims 79-95, wherein one week after administration to a human subject, a PMBC of the human subject has a tenofovir-diphosphate concentration of greater than 1 μM.

98. The pharmaceutical formulation of any one of claims 79-95, wherein two weeks after administration to a human subject, a PMBC of the human subject has a tenofovir-diphosphate concentration of greater than 1 μM.

99. The pharmaceutical formulation of any one of claims 79-95, wherein three weeks after administration to a human subject, a PMBC of the human subject has a tenofovir-diphosphate concentration of greater than 1 μM.

100. The pharmaceutical formulation of any one of claims 79-95, wherein one month after administration to a human subject, a PMBC of the human subject has a tenofovir-diphosphate concentration of greater than 1 μM.

101. The pharmaceutical formulation of any one of claims 79-95, wherein two month after administration to a human subject, a PMBC of the human subject has a tenofovir-diphosphate concentration of greater than 1 μM.

102. The pharmaceutical formulation of any one of claims 79-95, wherein three month after administration to a human subject, a PMBC of the human subject has a tenofovir-diphosphate concentration of greater than 1 μM.

103. The pharmaceutical formulation of any one of claims 79-102, wherein the pharmaceutical formulation is for administration by a 19 G needle or a 20 G needle.

104. The pharmaceutical formulation of any one of claims 79-103, wherein a dose of the pharmaceutical formulation is about 2 mL to about 3 mL of the pharmaceutical formulation.

105. The pharmaceutical formulation of any one of claims 79-104, wherein the pharmaceutical formulation is for administration at a frequency of once in a month or less.

106. The pharmaceutical formulation of any one of claims 79-105, wherein the pharmaceutical formulation is for administration at a frequency of once in two months or less.

107. The pharmaceutical formulation of any one of claims 79-106, wherein the pharmaceutical formulation is for administration at a frequency of once in three months or less.

108. The pharmaceutical formulation of any one of claims 79-107, wherein a dose of the pharmaceutical formulation delivers about 50-150 mg of the compound of Formula I, or the pharmaceutically acceptable salt thereof.

109. A method for treating a human immunodeficiency virus (HIV) infection, the method comprising administering to a human subject in need thereof the pharmaceutical composition of any one of claims 1-78, or the pharmaceutical formulation of any one of claims 79-108.

110. A method of treating an HBV infection, comprising administering to a human subject in need thereof the pharmaceutical composition of any one of claims 1-78, or the pharmaceutical formulation of any one of claims 79-108.

111. The method of claim 109 or 110, wherein the pharmaceutical composition or the pharmaceutical formulation is administered once a month or less.

112. The method of any one of claims 109-111, wherein the pharmaceutical composition or the pharmaceutical formulation is administered at a frequency of once in two months or less.

113. The method of any one of claims 109-112, wherein the pharmaceutical composition or the pharmaceutical formulation is administered at a frequency of once in three months or less.

114. The method of any one of claims 109-113, further comprising administering another therapeutic agent selected from the group consisting of HIV protease inhibiting compounds, HIV nonnucleoside inhibitors of reverse transcriptase, HIV nucleoside inhibitors of reverse transcriptase, HIV nucleotide inhibitors of reverse transcriptase, HIV integrase inhibitors, and CCR5 inhibitors.

115. A method of making the pharmaceutical composition of any one of claims 1-78, the method comprising:
(i) mixing the compound of Formula I: 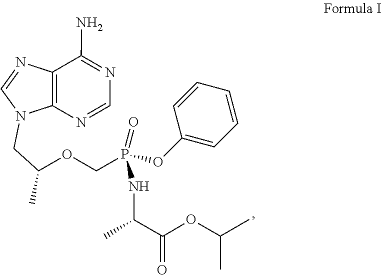 [SMILES: CC(C)OC(=O)[C@H](C)N[P@](=O)(CO[C@H](C)Cn1cnc2c(N)ncnc21)Oc1ccccc1]
or the pharmaceutically acceptable salt thereof, and
the biodegradable polymer, and
(ii) hot melt extrusion of a mixture comprising the compound of Formula I, or the pharmaceutically acceptable salt thereof, and the biodegradable polymer.

116. The method of claim 115, further comprising pelletization of an extruded composition comprising the compound of Formula I, or the pharmaceutically acceptable salt thereof, and the biodegradable polymer to obtain pellets comprising the compound of Formula I, or the pharmaceutically acceptable salt thereof, and the biodegradable polymer.

117. The method of claim 116, wherein the pellets have a size (diameter) of about 1 mm to about 3 mm.

118. The method of claim 116 or 117, further comprising micronizing the pellets comprising the compound of Formula I, or the pharmaceutically acceptable salt thereof, and the biodegradable polymer to obtain particles comprising the compound of Formula I, or the pharmaceutically acceptable salt thereof, and the biodegradable polymer.

119. The method of claim 118, further comprising classifying by size the particles comprising the compound of Formula I, or the pharmaceutically acceptable salt thereof, and the biodegradable polymer.

120. The method of any one of claims 115-119, further comprising mixing an additional agent with the compound of Formula I, or the pharmaceutically acceptable salt thereof, and the biodegradable polymer.

121. The method of claim 120, wherein the additional agent is a therapeutic agent.

122. The method of claim 120 or 121, wherein the additional agent is an anti-inflammatory agent.

123. The method of any one of claims 120-122, wherein the additional agent is a steroid.

124. The method of any one of claims 120-123, wherein the additional agent is and a corticosteroid.

125. The method of any one of claims 120-124, wherein the additional agent is dexamethasone.

126. The method of any one of claims 115-125, wherein the pharmaceutical composition has a d₁₀ value of greater than about 70-50 μm.

127. The method of any one of claims 115-126, wherein the pharmaceutical composition has a d₁₀ value of about 60 μm.

128. The method of any one of claims 115-127, wherein the pharmaceutical composition has a d₉₀ value of less than about 250 μm.

129. The method of any one of claims 115-128, wherein the pharmaceutical composition has a d₉₀ value of about 120-200 μm.

130. The method of any one of claims 115-129, wherein the pharmaceutical composition has a d₉₀ value of about 130-160 μm.

131. The method of any one of claims 115-130, wherein the pharmaceutical composition has a d₅₀ value 100-80 μm.

132. The method of any one of claims 115-131, wherein the pharmaceutical composition has a d₅₀ value of about 90 μm.

133. The method of any one of claims 115-132, wherein the pharmaceutical composition has a d₉₀ value of about 132 μm, a d₅₀ value of about 90 μm, and a d₁₀ value of about 60 μm.

134. The method of any one of claims 115-133, wherein the biodegradable polymer is PLGA.

135. A pharmaceutical composition prepared by the methods of any one of claims 115-134.
